(19) United States
(12) Patent Application Publication
(10) Pub. No.: US 2024/0101584 A1
(43) Pub. Date: Mar. 28, 2024

(54) TARGETED DELIVERY OF 1,2,4,5 TETRAOXANE COMPOUNDS AND THEIR USES

(71) Applicant: Westlake University, Hangzhou, Zhejiang (BN)

(72) Inventors: Dan YANG, Hangzhou Zhejiang (CN); Ming Kwan WONG, Kowloon (HK)

(73) Assignee: Westlake University, Hangzhou, Zhejiang (CN)

(21) Appl. No.: 18/266,936

(22) PCT Filed: Jan. 29, 2022
(86) PCT No.: PCT/CN2022/074975

§ 371 Date: Jun. 13, 2023

Related U.S. Application Data
(60) Provisional application No. 63/144,322, filed on Feb. 1, 2021.

(51) Int. Cl. C07F 9/655; A61K 45/06; A61P 35/00; C07D 323/04; C07D 405/12; C07D 407/12; C07H 13/12

(52) U.S. Cl. CPC C07F 9/65522; A61K 45/06; A61P 35/00; C07D 323/04; C07D 405/12; C07D 407/12; C07H 13/12

(57) ABSTRACT

1, 2, 4, 5-tetraoxane compounds and derivatives thereof that have anticancer properties are disclosed. Pharmaceutical compositions and pharmaceutical formulations in unit dosage form suitable for the delivery of the compounds to a subject in need thereof are disclosed. The pharmaceutical compositions or formulations may include one or more active agents in addition to the compounds, such as one or more additional anticancer agents. Methods for treating a cancer, reducing a cancer, or treating or ameliorating one or more symptoms associated with a cancer in a subject are also disclosed. The methods include (i) administering to a subject in need thereof an effective amount of the compound (s). The compound (s) can be administered by oral administration, parenteral administration, inhalation, mucosal administration, or a combination thereof. The compound can selectively kill cancer cells and/or cancer stem cells over non-cancerous cells by triggering ferroptosis in the cancer cells and/or cancer stem cells.

### FIELD OF THE INVENTION

This invention is generally in the field of 1,2,4,5-tetraoxane compounds and their use as anticancer and anticancer stem cell agents.

### BACKGROUND OF THE INVENTION

The triple negative breast cancer (TNBC) MDA-MB-231 cells are more aggressive and difficult to kill than estrogen, progesterone and HER2-positive breast cancer cells. Since TNBC has no known targets, chemotherapy and radiotherapy are the only options for TNBC. However, patients usually suffer from the severe side effects of the therapy. Additionally, traditional therapies like radiotherapy and photodynamic therapy require external light sources for generating therapeutic reactive oxygen species to kill the cancers. These two methods suffer from the shallow penetration of laser and hypoxia condition from tumours, which result in a poor therapeutic efficiency.

Cancer cells and cancer stem cells (CSCs) are also capable of developing apoptosis resistance against some current existing drugs like Taxol and artesunate. As chemotherapy and radiotherapy kill cancer cells mainly by inducing apoptosis, these methods can develop resistance towards apoptotic cell death and thus fail to achieve therapeutic purpose. Additionally, CSCs are known to be resistant to chemotherapy or radiotherapy. CSCs have the ability of self-renewal. If a drug can treat cancer cells but not CSCs, the patient will have higher chance of cancer reoccurrence. If a drug can treat CSCs only, the patient still suffers from the disease as the cancer cells are present.

Ferroptosis is an iron-dependent and reactive oxygen species (ROS)-dependent cell death pathway. It is known that cancer cells have elevated level of iron, which favors ferroptosis to induce deleterious lipid peroxides and irreversible cell death, bypassing the anti-apoptotic pathways. However, the majority of ferroptosis inducers lack selectivity towards cancer cells compared with non-cancerous cells. For example, erastin and RSL3 are more cytotoxic to non-cancerous cells than cancer cells, showing that they have no selectivity towards cancer cells.

There remains a need to develop anticancer compounds that have selectivity towards cancer cells and cancer stem cells over non-cancerous cells.

Therefore, it is the object of the present invention to provide anticancer compounds.

It is another object of the present invention to provide methods of using the anticancer compounds.

### SUMMARY OF THE INVENTION

1,2,4,5-tetraoxane compounds and derivatives and methods of using the compounds for treating a cancer, reducing a cancer, or treating or ameliorating one or more symptoms associated with a cancer in a subject are described.

Generally, the compounds can have three moieties: a cyclic ring moiety, a 1,2,4,5-tetraoxane moiety, and a targeting moiety. In some forms, the compound can have the structure of Formula I.

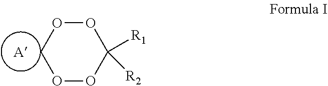

(a) where A′ can be a substituted or unsubstituted monocyclic ring, a substituted or unsubstituted polycyclic ring; (b) where R₁ and R₂ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, a phosphonyl, a silyl, a sulfinyl, a thiol, a hydroxyl, or R₁ and R₂ taken together with the carbon to which they are attached form a substituted or unsubstituted monocyclic ring, or a substituted or unsubstituted polycyclic ring; and (c) where the substituents can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group (such as a glucose group or an acetylated glucose), or a combination thereof.

In some forms, the compound can have the structure of Formula II.

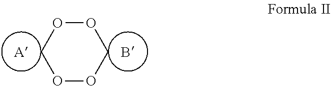

(a) where A′ and B′ can be independently a substituted or unsubstituted monocyclic ring, or a substituted or unsubstituted polycyclic ring, such as those described above for A′; and (b) where the substituents can be any substituents described above.

In some forms, the compound can have the structure of Formula III.

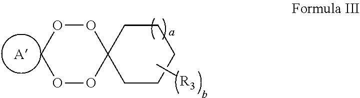

(a) where A′ can be as described above; (b) where a can be an integer from 1 to 20; (c) where b can be an integer from 0 to 24; and (d) where each occurrence of R₃ can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, a substituted or unsubstituted alkoxy, an aroxy, a cyano, an isocyano, a nitro, a substituted or unsubstituted carbonyl, an amino, an amido, a silyl, a sulfinyl, a substituted or unsubstituted sulfonyl, a phosphonium, a phosphanyl, a phosphoryl, a phosphonyl, a thiol, or a sugar group (such as a glucose group or an acetylated glucose); where the substituents can be any substituents described above.

In some forms, the compound can have the structure of Formula IV

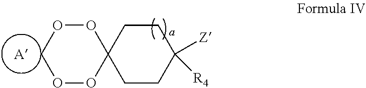

(a) where A′ and a can be as described above; and (b) where Z′ and R₄ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a sulfinyl, a sulfonyl, a phosphonium, a phosphanyl, a phosphoryl, a phosphonyl, a thiol, or a sugar group (such as a glucose group or an acetylated glucose); where the substituents can be any substituents described above.

In some forms, the compound can have the structure of Formula V.

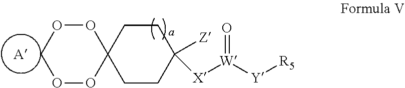

(a) where A′, a, and Z′ can be as described above; (b) where X′ can be

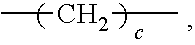

O, NR₆, or S, and c can be an integer from 0 to 30; (c) where W′ can be C, PR₇, S, or Si; (d) where Y′ can be NR₈ or O; (e) where R₆ and R₇ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl; and (f) where R₅ and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl, or R₅ and R₈ taken together with the Y′ to which they are attached form a substituted or unsubstituted cycloalkyl, a substituted or unsubstituted cycloalkenyl, a substituted or unsubstituted cycloalkynyl, a substituted or unsubstituted heterocyclyl, a substituted or unsubstituted aryl, a substituted or unsubstituted heteroaryl, a substituted or unsubstituted polyaryl, or a substituted or unsubstituted polyheteroaryl; where the substituents can be any substituents described above.

In some forms, the compound can have the structure of Formula VI.

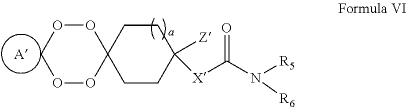

(a) where A′ and a can be as described above; (b) where Z′ can be a hydrogen, substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a phosphonium, or a sugar group (such as a glucose group or an acetylated glucose); (c) where X′ can be

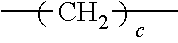

or O, and c can be an integer from 0 to 10; (d) where R₅ and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, or R₅ and R₈ taken together with the nitrogen to which they are attached form a substituted or unsubstituted cycloalkyl, a substituted or unsubstituted cycloalkenyl, a substituted or unsubstituted cycloalkynyl, a substituted or unsubstituted heterocycloalkyl, a substituted or unsubstituted heterocycloalkenyl, a substituted or unsubstituted heterocycloalkynyl, a substituted or unsubstituted aryl, a substituted or unsubstituted heteroaryl, a substituted or unsubstituted polyaryl, or a substituted or unsubstituted polyheteroaryl.

In some forms, the compound can have the structure of Formula VII.

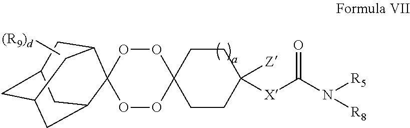

(a) where a, X′, R₅, R₈ can be as described above; (b) where Z′ can be a hydrogen or a substituted or unsubstituted alkyl; and (c) where R₉ can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, and d is an integer from 0 to 9; where the substituents can be any substituents described above.

In some forms, the compound can have the structure of Formula VIII.

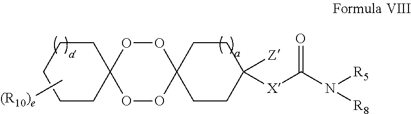

(a) where a, X′, R₅, and R₈ can be as described above; (b) where a′ can be an integer from 1 to 20; (c) where Z′ can be a hydrogen or a substituted or unsubstituted alkyl; and (d) where R₁₀ can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, and e is an integer from 0 to 24; where the substitutes can be any substituents described above.

In some forms, the compound can have the structure of Formula IX.

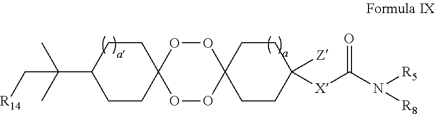

(a) where a, a′, Z, X′, R₅, and R₈ can be as described above; and (b) where R₁₄ can be a hydrogen, a substituted or unsubstituted alkenyl, a substituted or unsubstituted alkynyl, a substituted or unsubstituted carbonyl, a substituted or unsubstituted alkoxyl, or an amido.

The compound can have the structure of Formula X.

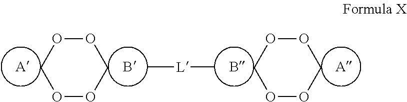

(a) where A′ and A″ can be independently a substituted or unsubstituted monocyclic ring, a substituted or unsubstituted polycyclic ring; (b) where L′ can be a linker, such as an oxygen, a sulfur, a carbon, a boron, a substituted or unsubstituted alkoxy, a substituted or unsubstituted alkyl, a substituted or unsubstituted alkenyl, a substituted or unsubstituted cycloalkyl, a substituted or unsubstituted cycloalkenyl, a substituted or unsubstituted aryl, a substituted or unsubstituted heterocycloalkyl, a substituted or unsubstituted heteroaryl, a phosphoryl, a sulfinyl, a sulfonyl, an ether, a polyether, a disulfide, and an amino; and (c) where the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group (such as a glucose group or an acetylated glucose), or a combination thereof.

In some forms, the compound can have the structure of Formula XI.

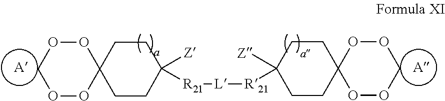

(a) where A′, A″, and L′ can be as described above; (b) where a and a″ can be independently an integer from 1 to 20, from 1 to 15, from 1 to 10, from 1 to 5, from 1 to 3, or 1 or 2; (c) where Z′, Z″, R₂₁, and R′₂₁ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a sulfinyl, a sulfonyl, a phosphonium, a phosphanyl, a phosphoryl, a phosphonyl, a thiol, or a sugar group (such as a glucose group or an acetylated glucose); where the substituents can be any substituents described above.

In some forms, the compound can have the structure of Formula XII.

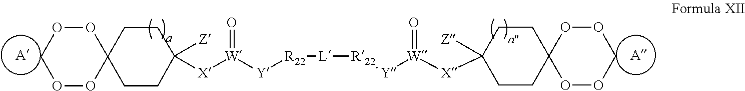

(a) where A′, A″, U, a, a″, Z′, and Z″ can be as described above; (b) where X′ and X″ can be independently

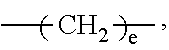

O, NR₆, or S, and c can be an integer from 0 to 30, from 0 to 25, from 0 to 20, from 0 to 15, from 0 to 10, from 0 to 5, from 0 to 3, or 0, 1, or 2; (c) where W′ and W″ can be independently C, PR₇, S, or Si; (d) where Y′ and Y″ can be independently a bond or is NR₈ or O; (e) where R₂₂ and R′₂₂ can be independently

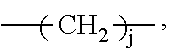

and j is an integer from 0 to 20, from 0 to 15, from 0 to 10, from 0 to 5, from 0 to 3, or 0, 1, or 2; (f) where R₆, R₇, and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl; where the substituents can be any substituents described above.

Pharmaceutical compositions and pharmaceutical formulations in unit dosage form suitable for the delivery of the compounds and their preparation are disclosed. Generally, the pharmaceutical composition or formulation contains the compound(s) and a pharmaceutically acceptable excipient. The compound(s) in the pharmaceutical compositions or formulations is in an effective amount for treating a cancer, reducing a cancer, or treating or ameliorating one or more symptoms associated with a cancer in a subject. In some forms, the pharmaceutical composition or formulation can further contain one or more active agents in addition to the compounds, such as one or more additional anticancer agents.

The methods of using the compounds include (i) administering to the subject an effective amount of the compound(s) to treat the cancer, reduce the cancer, or treat or ameliorate one or more symptoms associated with the cancer in the subject. The subject is typically a mammal, such as a human. In some forms, the cancer being treated or reduced can be colon cancer, breast cancer, ovarian cancer, cervical cancer, lung cancer, rectal cancer, kidney cancer, liver cancer, brain cancer, or leukemia, or a combination thereof. The compound(s) can be administered by oral administration, parenteral administration, inhalation, mucosal, topical administration, or a combination thereof. In some forms, the methods can further include administering to the subject one or more second active agents, such as additional anticancer agent(s), prior to, during, and/or subsequent to step (i).

Methods for treating cancer cells and/or cancer stem cells in a subject in need thereof are also disclosed. The method includes administering to the subject an effective amount of the compound, where the compound has an IC₅₀ value against the cancer cells lower than IC₅₀ value of the same compound against non-cancerous cells, tested under the same condition, and/or where the compound has an IC₅₀ value against the cancer cells or cancer stem cells lower than an IC₅₀ value of a known compound (e.g. OZ277, OZ439, RKA182, OZ277, OZ439, RKA182, FINO₂, or a cholic acid/deoxycholic acid/steroid derivative of 1,2,4,5-tetraoxane) against the same cancer cells or cancer stem cells, tested under the same condition. In some forms, the cancer cells being treated can be MDA-MB-231 cells, MCF7 cells, Hela cells, T47D cells, Huh7 cells, PLC cells, U20S cells, HEK293 cells, HepG2 cells, Jurkat cells, HCT116 cells, HEYA8 cells, or HL-60 cells, or a combination thereof; and/or the non-cancerous cells can be NIH3T3 cells, MDCK cells, or bEnd.3 cells, or a combination thereof. In some forms, the compound induces ferroptosis in the cancer cells and/or cancer stem cells, optionally where the intracellular pH of the cancer cells and/or cancer stem cells is in a range from 6 to 7.5.

### BRIEF DESCRIPTION OF THE DRAWINGS

FIG. 1 is a schematic showing the biological features of an exemplary compound.

FIG. 2 is schematic showing the procedure of in vivo experiment.

FIGS. 3A-3B are graphs showing the results of three independent experiments for dose-dependent viability curves of HeyA8, HeyA8 CSCs, and NIH3T3 in response to the treatment of compounds 37b (FIG. 3A) and 48 (FIG. 3B) for 48 hrs (mean±SD, n=3). FIG. 3C is a bar graph showing that compounds 37b and 48 could diminish the cell viability of both ovarian cancer cells and cancer stem cells (CSCs). *P≤0.05 versus drug treated control. Data are plotted as the mean±SD, n=3 biologically independent samples. All P values were determined using two-tailed Student's t-test.

FIG. 4 is a bar graph showing the protective effect of DFO and Fer-1 on HeyA8 cancer cells after treatment with compound 48 for 24 h. *P≤0.01, ***P≤0.001 versus non-pretreated group. Data represent mean±SD, n=3 biologically independent samples. All P values were determined using two-tailed Student's t-test.

FIGS. 5A-5B are bar graphs showing the protective effect of Q-VD-OPh, deferasirox (DFX) and ferrostatin-1 (Fer-1) on HeyA8 cancer stem cells (FIG. 5A) and HeyA8 spheres (FIG. 5B) after treatment with compound 48 and compound 37b, respectively, for 24 h. *P≤0.05, **P≤0.01 versus non-pretreated group. Data are plotted as the mean±SD, n=3 biologically independent samples. N.S., no statistically significant change. All P values were determined using two-tailed Student's t-test.

FIG. 6 is a bar graph showing the effect of erastin, RSL3, and artesunate on the cell viability of both ovarian cancer cells and cancer stem cells (CSCs). *P≤0.05 and **P≤0.01 versus DMSO control. Data are plotted as the mean±SD, n=3 biologically independent samples. All P values were determined using two-tailed Student's t-test.

FIG. 7A is a graph showing the tumor volume of xenograft tumors versus time after s.c. injection of DMSO, PTX, compound 37b, or compound 48. Nude mice were injected s.c. with HEYA8 CSC cells (10⁶) and randomly allocated to one of the six treatment groups (n=4): (i) DMSO, (ii) Paclitaxel (PTX), 5 mg/kg, (iii) compound 37b, 5 mg/kg, (iv) compound 37b, 10 mg/kg, (v) compound 48, 5 mg/kg, or (vi) compound 48, 10 mg/kg. When control animals (DMSO) began to appear tumor ulceration (25 days), all animals in the experiment were sacrificed and photographed. FIG. 7B are photographs of the tumor from sacrificed animals of different treatment groups after 25 days. FIG. 7C is a graph showing the tumor weight of xenograft tumors versus time after s.c. injection of DMSO, PTX, compound 37b, or compound 48. Data are shown as mean±SEM. * P≤0.05 versus DMSO group.

FIG. 8A is a bar graph showing the effect of Fer-1 (50 μM) on MDA-MB-231 cells treated with compounds 37b, 37a, and 30b, respectively, at the indicated concentrations for 24 h. *P≤0.5, **P≤0.01, ***P≤0.001 versus non-pretreated cells, N.S., no statistically significant change. Data represent mean±SD, n=3 biologically independent samples. All P values were determined using two-tailed Student's t-test.

FIG. 8B is a bar graph showing the effect of Fer-1 (50 μM) on MDA-MB-231 cells treated with compounds 6b, 48, 30a, and 37f, respectively, at the indicated concentrations for 24 h. *P≤0.01, **P≤0.002, ***P≤0.001 versus non-pretreated cells. Data represent mean±SD, n=3 biologically independent samples. FIG. 8C is a bar graph showing the effect of Fer-1 and DFO on MDA-MB-231 cells treated with compounds 56 and 53, respectively, at the indicated concentrations for 24 h. *P≤0.01, **P≤0.002, ***P≤0.001 versus non-pretreated cells. All P values were determined using two-tailed Student's t-test.

FIG. 9 is a bar graph showing the effect of liproxstatin (30 PM) on MDA-MB-231 cells treated with compounds 48 and 37b, respectively, at the indicated concentrations for 24 h. **P≤0.01 versus non-pretreated cells. Data represent mean±SD, n=3 biologically independent samples. All P values were determined using two-tailed Student's t-test.

FIG. 10 is a bar graph showing the relative mean fluorescence intensities of cells with HKOH-1r measured from the confocal images of MDA-MB-231 cells after treatment with compounds 48 and 37b, respectively. Scale bars represent 50 μm. Data are mean±s.e.m., n=20 cells for HKOH-1. Statistical significance was determined as ***P≤0.01 by Student's t test.

FIG. 11 is a bar graph showing the detection of lipid ROS with compound 48 (5 μM), compound 37b (1 μM), or RSL3 (0.5 μM) by BODIPY 581/591 C11 after 6 h incubation in MDA-MB-231 cells. **P≤0.01 versus DMSO group. Data are plotted as the mean±SD, n=3 biologically independent samples. All P values were determined using two-tailed Student's t-test.

FIG. 12A is a graph showing the tumor volume of xenograft tumors versus time after s.c. injection of DMSO, PTX, compound 37b, or compound 48. Nude mice were injected s.c. with MDA-MB-231 cells (10⁶) and randomly allocated to one of the six treatment groups (n=5): (i) DMSO, (ii) Paclitaxel (PTX), 5 mg/kg, (iii) compound 37b, 5 mg/kg, (iv) compound 37b, 10 mg/kg, (v) compound 48, 5 mg/kg, or (vi) compound 48, 10 mg/kg. When control animals (DMSO) began to appear tumor ulceration (25 days), all animals in the experiment were sacrificed and photographed.

FIG. 12B are photographs of the tumor from sacrificed animals of different treatment groups after 25 days. FIG. 12C is a graph showing the tumor weight of xenograft tumors versus time after s.c. injection of DMSO, PTX, compound 37b, or compound 48. Data are shown as mean±SEM. * P≤0.05 versus DMSO group. FIG. 12D are photographs showing the morphological observation of mice tissue treated with DMSO, paclitaxel, compound 37b, or compound 48 with H&E staining on heart, liver, kidney, lungs, and spleen tissues (200×).

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid, poly(lactic-co-glycolic acid), a peptide, a polypeptide group, and a sugar group (such as a glucose group, an acetylated glucose, a fructose, an acetylated fructose, etc.). Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid, poly(lactic-co-glycolic acid), a peptide, a polypeptide group, and a sugar group can be further substituted.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

The term “alkyl” as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety; —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof. The term “alkyl” also includes “heteroalkyl”.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl”.

The term “substituted alkenyl” refers to alkenyl moieties having one or more substituents replacing one or more hydrogen atoms on one or more carbons of the hydrocarbon backbone. Such substituents can be any substituents described above.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl”.

The term “substituted alkynyl” refers to alkynyl moieties having one or more substituents replacing one or more hydrogen atoms on one or more carbons of the hydrocarbon backbone. Such substituents can be any substituents described above.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

The term “aryl” as used herein is any C₅-C₂₆ carbon-based aromatic group, heteroaromatic, fused aromatic, or fused heteroaromatic. For example, “aryl,” as used herein can include 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused aromatic rings”), wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents. Such substituents can be any substituents described above.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₃₀-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl”.

The term “substituted heteroaryl” refers to a heteroaryl group in which one or more hydrogen atoms on one or more heteroaromatic rings are substituted with one or more substituents. Such substituents can be any substituents described above.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof. For example, a “polyaryl” can be polycyclic ring systems having two or more cyclic rings in which two or more carbons are common to two adjoining rings (i.e., “fused aromatic rings”), wherein two or more of the rings are aromatic. When two or more heteroaryls are involved, the chemical moiety can be referred to as a “polyheteroaryl”.

The term “substituted polyaryl” refers to a polyaryl in which one or more of the aryls, heteroaryls are substituted, with one or more substituents. Such substituents can be any substituents described above. When two or more heteroaryls are involved, the chemical moiety can be referred to as a “substituted polyheteroaryl.”

The term “cyclic ring” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, a substituted or unsubstituted heterocyclyl, a substituted or unsubstituted aryl, a substituted or unsubstituted polyaryl, a substituted or unsubstituted heteroaryl, and a substituted or unsubstituted polyheteroaryl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, heterocyclyls, aryls, heteroaryl, polyaryls, and polyheteroaryls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a sugar group, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-aryl, —O-heteroaryl, —O— polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above.

The term “ether” as used herein is represented by the formula A²OA¹, where A² and A can be, independently, a sugar group, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

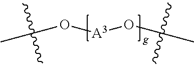

where A³ can be a sugar group, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized, and refers to a compound of the formula —OR^{v} wherein R^{v} is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above.

The term “amino” as used herein includes the group

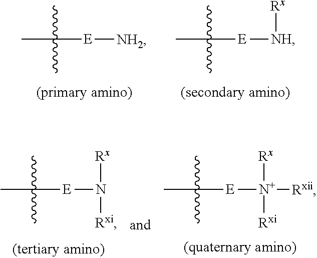

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure.

The terms “amide” or “amido” are used interchangeably, refer to both “unsubstituted amido” and “substituted amido” and are represented by the general formula:

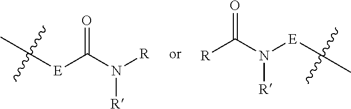

wherein, E is defined as above for “amino”, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ and m are defined as above for “amino”. In some forms, when E is oxygen, a carbamate is formed.

“Carbonyl,” as used herein, is art-recognized and includes such moieties as can be represented by the general formula:

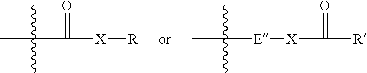

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Where X is oxygen and R is defines as above, the moiety is also referred to as a carboxyl group. When X is oxygen and R is hydrogen, the formula represents a “carboxylic acid”. Where X is oxygen and R′ is hydrogen, the formula represents a “formate”. Where X is oxygen and R or R′ is not hydrogen, the formula represents an “ester”. In general, where the oxygen atom of the above formula is replaced by a sulfur atom, the formula represents a “thiocarbonyl” group. Where X is sulfur and R or R′ is not hydrogen, the formula represents a “thioester”. Where X is sulfur and R is hydrogen, the formula represents a “thiocarboxylic acid”. Where X is sulfur and R′ is hydrogen, the formula represents a “thioformate”. Where X is a bond and R is not hydrogen, the above formula represents a “ketone”. Where X is a bond and R is hydrogen, the above formula represents an “aldehyde”.

The term “substituted carbonyl” refers to a carbonyl, as defined above, wherein one or more hydrogen atoms in R, R′ or a group to which the moiety

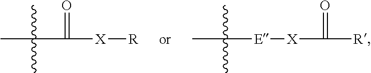

is attached, are independently substituted. Such substituents can be any substituents described above.

The term “carboxyl” is as defined above for carbonyl and is defined more specifically by the formula —R^{iv}COOH, wherein R^{iv} is a substituted or unsubstituted alkyl, a substituted or unsubstituted alkenyl, a substituted or unsubstituted alkynyl, a substituted or unsubstituted heterocyclyl, a substituted or unsubstituted alkylaryl, a substituted or unsubstituted arylalkyl, a substituted or unsubstituted aryl, a substituted or unsubstituted polyaryl, a substituted or unsubstituted polyheteroaryl, or a substituted or unsubstituted heteroaryl.

The term “substituted carboxyl” refers to a carboxyl, as defined above, wherein one or more hydrogen atoms in R^{iv} are substituted. Such substituents can be any substituents described above.

The term “phosphanyl” is represented by the formula

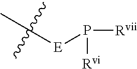

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′, or R^{vi} and R^{vii} taken together with the P atom to which they are attached complete a heterocycle having from 3 to 14 atoms in the ring structure; R′″ and m are defined as above for “amino”.

The term “phosphonium” is represented by the formula

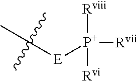

wherein, E is defined as above for “phosphanyl”, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ and m are defined as above for “amino”.

The term “phosphonyl” is represented by the formula

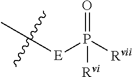

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ and m are defined as above for “amino”.

The term “substituted phosphonyl” represents a phosphonyl in which E, R^{vi} and R^{vii} are independently substituted. Such substituents can be any substituents described above.

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art.

The term “sulfinyl” is represented by the formula

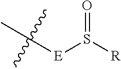

wherein E is defined as above for “phosphonyl”, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ and m are defined as above for “amino”.

The term “sulfonyl” is represented by the formula

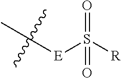

wherein E is defined as above for “phosphonyl”, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ and m are defined as above for “amino”.

The term “substituted sulfonyl” represents a sulfonyl in which E, R, or both, are independently substituted. Such substituents can be any substituents described above.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

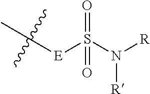

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ and m are defined as above for “amino”.

The term “silyl group” as used herein is represented by the formula —SiRR′R″, where R, R′, and R″ can be, independently, a hydrogen, a sugar group, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above.

The terms “thiol” are used interchangeably and are represented by —SR, where R can be a hydrogen, a sugar group, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto. The term “substituted alkylthio” refers to an alkylthio group having one or more substituents replacing one or more hydrogen atoms on one or more carbon atoms of the alkylthio backbone.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring.

“Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein.

The terms “hydroxyl” and “hydroxy” are used interchangeably and are represented by —OH.

The term “oxo” refers to ═O bonded to a carbon atom.

The terms “cyano” and “nitrile” are used interchangeably to refer to —CN.

The term “nitro” refers to —NO₂.

The term “phosphate” refers to —O—PO₃.

The term “azide” or “azido” are used interchangeably to refer to —N₃.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

### II. Compositions

1,2,4,5-tetraoxane compounds and their derivatives (together also referred to herein as “compounds”) having anticancer properties against cancer cells and/or cancer stem cells have been developed. These have broad anticancer properties and should be suitable for use in the treatment of multiple types of cancers and/or amelioration of the symptom(s) of multiple types of cancers.

Generally, the compounds contain three moieties:

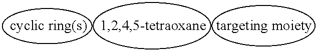

Without being bound by theory, the cyclic ring(s) moiety of the compounds can provide lipophilicity for cell permeability and maintain the compounds' lethality to cancer cells and/or cancer stem cells; and the targeting moiety provides water solubility, cytotoxicity, and selectivity towards the cancer cells and/or cancer stem cells over non-cancerous cells. For example, the targeting moiety can localize the compound to a specific position inside the cancer cells and/or cancer stem cells to enhance the potency and minimize the off-target effect, i.e. binding with the non-cancerous cells.

The overall structure of these compounds render them useful in triggering ferroptosis to kill cancer cells and/or cancer stem cells. The term “cancer cells” refers to cells with abnormal growth and division with the potential of invasiveness. A tumor contains cancer cells that have unregulated growth and promote construction of blood vessels. The term “cancer stem cells” refers to tumor-initiating cells, which are characterized by the abilities of self-renewal, differentiations, and chemoresistance. Without being bound to theories, cancer cells are different from cancer stem cells in at least the following aspects. For example, wnt/β-catenin, Notch, or Hedgehog signaling pathway can have more responsibility in regulating the growth and development of cancer stem cells than cancer cells. Cancer stem cells can be positive for stem cell surface markers like CD133, CD117, Bmi-1, Nanog, Sox4, CD44, etc. Cancer stem cells can overexpress ABC drug transporters to efflux chemotherapeutic drugs, such as paclitaxel and doxorubicin, compared with cancer cells. Cancer stem cells can belong to side population cells that exclude the DNA-specific dye by Hoechst 33342. Ferroptosis is an iron-dependent and reactive oxygen species (ROS)-dependent cell death pathway. It is well-known that cancer cells have elevated level of iron, which favors ferroptosis to induce deleterious lipid peroxides and irreversible cell death. The compounds can also selectively trigger ferroptosis in cancer cells and cancer stem cells compared with non-cancerous cells. Additionally, the compounds can generate reactive oxygen species inside the cancer cells and cancer stem cells regardless of the pH (e.g., generate hydroxyl radicals and lipid peroxides under neutral pH), eliminating complex formulations that encapsulate multiple components (e.g., enzymes that catalyze hydrogen peroxide production, iron oxide, etc.) and the requirement of acidic intracellular environment in chemodynamic therapy, i.e. ferroptosis.

Pharmaceutical compositions and formulations containing the compounds are also disclosed.

A. Compounds

### 1. 1,2,4,5-Tetraoxanes and Derivatives Thereof

The compounds can have the structures of Formula I.

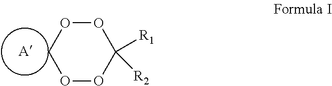

(a) where A′ can be a substituted or unsubstituted monocyclic ring, a substituted or unsubstituted polycyclic ring; and (b) where R₁ and R₂ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, a phosphonyl, a silyl, a sulfinyl, a thiol, a hydroxyl, or R₁ and R₂ taken together with the carbon to which they are attached form a substituted or unsubstituted monocyclic ring, or a substituted or unsubstituted polycyclic ring. E.

Exemplary substituents can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group (such as a glucose group or an acetylated glucose), or a combination thereof.

In some forms, A′ can be a substituted or unsubstituted cycloalkyl, a substituted or unsubstituted cycloalkenyl, a substituted or unsubstituted cycloalkynyl, a substituted or unsubstituted heterocyclyl (such as a substituted or unsubstituted heterocycloalkyl, a substituted or unsubstituted heterocycloalkenyl, a substituted or unsubstituted heterocycloalkynyl), a substituted or unsubstituted aryl, a substituted or unsubstituted polyaryl, a substituted or unsubstituted heteroaryl, and a substituted or unsubstituted polyheteroaryl, that have from three to 30 carbon atoms, as geometric constraints permit.

The alkyl can be a linear alkyl, a branched alkyl, or a cyclic alkyl (either monocyclic or polycyclic). Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, C₁-C₂ alkyl group, a branched C₃-C₉, C₃-C₉, C₃-C₅, C₃-C₇, C₃-C₆, C₃-C₅, C₃-C₄ alkyl group, or a cyclic C₃-C₉, C₃-C₉, C₃-C₅, C₃-C₇, C₃-C₆, C₃-C₅, C₃-C₄ alkyl group.

The alkenyl can be a linear alkenyl, a branched alkenyl, or a cyclic alkenyl (either monocyclic or polycyclic). Exemplary alkenyl include a linear C₁-C₃₀ alkenyl, a branched C₄-C₃₀ alkenyl, a cyclic C₃-C₃₀ alkenyl, a linear C₁-C₂₀ alkenyl, a branched C₄-C₂₀ alkenyl, a cyclic C₃-C₂₀ alkenyl, a linear C₁-C₁₀ alkenyl, a branched C₄-C₁₀ alkenyl, a cyclic C₃-C₁₀ alkenyl, a linear C₁-C₆ alkenyl, a branched C₄-C₆ alkenyl, a cyclic C₃-C₆ alkenyl, a linear C₁-C₄ alkenyl, cyclic C₃-C₄ alkenyl, such as a linear C₁-C₁₀, C₁-C₉, C₁-C₈, C₁-C₇, C₁-C₆, C₁-C₅, C₁-C₄, C₁-C₃, C₁-C₂ alkenyl group, a branched C₃-C₉, C₃-C₉, C₃-C₅, C₃-C₇, C₃-C₆, C₃-C₅, C₃-C₄ alkenyl group, or a cyclic C₃-C₉, C₃-C₉, C₃-C₅, C₃-C₇, C₃-C₆, C₃-C₅, C₃-C₄ alkenyl group.

The alkynyl can be a linear alkynyl, a branched alkynyl, or a cyclic alkynyl (either monocyclic or polycyclic). Exemplary alkynyl include a linear C₁-C₃₀ alkynyl, a branched C₄-C₃₀ alkynyl, a cyclic C₃-C₃₀ alkynyl, a linear C₁-C₂₀ alkynyl, a branched C₄-C₂₀ alkynyl, a cyclic C₃-C₂₀ alkynyl, a linear C₁-C₁₀ alkynyl, a branched C₄-C₁₀ alkynyl, a cyclic C₃-C₁₀ alkynyl, a linear C₁-C₆ alkynyl, a branched C₄-C₆ alkynyl, a cyclic C₃-C₆ alkynyl, a linear C₁-C₄ alkynyl, cyclic C₃-C₄ alkynyl, such as a linear C₁-C₁₀, C₁-C₉, C₁-C₈, C₁-C₇, C₁-C₆, C₁-C₅, C₁-C₄, C₁-C₃, C₁-C₂ alkynyl group, a branched C₃-C₉, C₃-C₉, C₃-C₅, C₃-C₇, C₃-C₆, C₃-C₅, C₃-C₄ alkynyl group, or a cyclic C₃-C₉, C₃-C₉, C₃-C₅, C₃-C₇, C₃-C₆, C₃-C₅, C₃-C₄ alkynyl group.

It is understood that any of the exemplary alkyl, alkenyl, and alkynyl groups can be heteroalkyl, heteroalkenyl, and heteroalkynyl, respectively. For example, the alkyl can be a linear C₂-C₃₀ heteroalkyl, a branched C₄-C₃₀ heteroalkyl, a cyclic C₃-C₃₀ heteroalkyl (i.e. a heterocycloalkyl), a linear C₁-C₂₀ heteroalkyl, a branched C₄-C₂₀ heteroalkyl, a cyclic C₃-C₂₀ heteroalkyl, a linear C₁-C₁₀ heteroalkyl, a branched C₄-C₁₀ heteroalkyl, a cyclic C₃-C₁₀ heteroalkyl, a linear C₁-C₆ heteroalkyl, a branched C₄-C₆ heteroalkyl, a cyclic C₃-C₆ heteroalkyl, a linear C₁-C₄ heteroalkyl, cyclic C₃-C₄ heteroalkyl, such as a linear C₁-C₁₀, C₁-C₉, C₁-C₈, C₁-C₇, C₁-C₆, C₁-C₅, C₁-C₄, C₁-C₃, C₁-C₂ heteroalkyl group, a branched C₃-C₉, C₃-C₉, C₃-C₅, C₃-C₇, C₃-C₆, C₃-C₅, C₃-C₄ heteroalkyl group, or a cyclic C₃-C₉, C₃-C₉, C₃-C₈, C₃-C₇, C₃-C₆, C₃-C₅, C₃-C₄ heteroalkyl group.

The alkenyl can be a linear C₂-C₃₀ heteroalkenyl, a branched C₄-C₃₀ heteroalkenyl, a cyclic C₃-C₃₀ heteroalkenyl (i.e. a heterocycloalkenyl), a linear C₁-C₂₀ heteroalkenyl, a branched C₄-C₂₀ heteroalkenyl, a cyclic C₃-C₂₀ heteroalkenyl, a linear C₁-C₁₀ heteroalkenyl, a branched C₄-C₁₀ heteroalkenyl, a cyclic C₃-C₁₀ heteroalkenyl, a linear C₁-C₆ heteroalkenyl, a branched C₄-C₆ heteroalkenyl, a cyclic C₃-C₆ heteroalkenyl, a linear C₁-C₄ heteroalkenyl, cyclic C₃-C₄ heteroalkenyl, such as a linear C₁-C₁₀, C₁-C₉, C₁-C₅, C₁-C₇, C₁-C₆, C₁-C₅, C₁-C₄, C₁-C₃, C₁-C₂ heteroalkenyl group, a branched C₃-C₉, C₃-C₉, C₃-C₅, C₃-C₇, C₃-C₆, C₃-C₅, C₃-C₄ heteroalkenyl group, or a cyclic C₃-C₉, C₃-C₉, C₃-C₅, C₃-C₇, C₃-C₆, C₃-C₅, C₃-C₄ heteroalkenyl group.

The alkynyl can be a linear C₂-C₃₀ heteroalkynyl, a branched C₄-C₃₀ heteroalkynyl, a cyclic C₃-C₃₀ heteroalkynyl (i.e. a heterocycloalkynyl), a linear C₁-C₂₀ heteroalkynyl, a branched C₄-C₂₀ heteroalkynyl, a cyclic C₃-C₂₀ heteroalkynyl, a linear C₁-C₁₀ heteroalkynyl, a branched C₄-C₁₀ heteroalkynyl, a cyclic C₃-C₁₀ heteroalkynyl, a linear C₁-C₆ heteroalkynyl, a branched C₄-C₆ heteroalkynyl, a cyclic C₃-C₆ heteroalkynyl, a linear C₁-C₄ heteroalkynyl, cyclic C₃-C₄ heteroalkynyl, such as a linear C₁-C₁₀, C₁-C₉, C₁-C₅, C₁-C₇, C₁-C₆, C₁-C₅, C₁-C₄, C₁-C₃, C₁-C₂ heteroalkynyl group, a branched C₃-C₉, C₃-C₉, C₃-C₅, C₃-C₇, C₃-C₆, C₃-C₅, C₃-C₄ heteroalkynyl group, or a cyclic C₃-C₉, C₃-C₉, C₃-C₅, C₃-C₇, C₃-C₆, C₃-C₅, C₃-C₄ heteroalkynyl group.

The aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. The polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

In some forms, the compound can have the structure of Formula II.

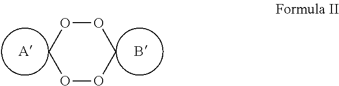

(a) where A′ and B′ can be independently a substituted or unsubstituted monocyclic ring, or a substituted or unsubstituted polycyclic ring, such as those described above for A′; and (b) where the substituents can be any substituents described above.

In some forms, the compound can have the structure of Formula III.

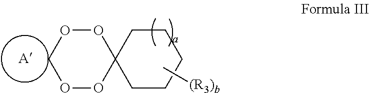

(a) where A′ can be as described above; (b) where a can be an integer from 1 to 20; (c) where b can be an integer from 0 to 24; and (d) where each occurrence of R₃ can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, a substituted or unsubstituted alkoxy, an aroxy, a cyano, an isocyano, a nitro, a substituted or unsubstituted carbonyl, an amino, an amido, a silyl, a sulfinyl, a substituted or unsubstituted sulfonyl, a phosphonium, a phosphanyl, a phosphoryl, a phosphonyl, a thiol, or a sugar group (such as a glucose group or an acetylated glucose); where the substituents can be any substituents described above.

In some forms of Formula III, a can be an integer from 1 to 15, from 1 to 12, from 1 to 10, from 1 to 8, from 1 to 5, or from 1 to 3, such as 1 or 2; and b is an integer from 1 to 10, from 1 to 8, from 1 to 5, or from 1 to 3, such as 1 or 2.

In some forms, the compound can have the structure of Formula IV

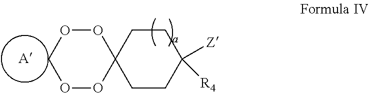

(a) where A′ and a can be as described above; and (b) where Z′ and R₄ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a sulfinyl, a sulfonyl, a phosphonium, a phosphanyl, a phosphoryl, a phosphonyl, a thiol, or a sugar group (such as a glucose group or an acetylated glucose); where the substituents can be any substituents described above.

In some forms of Formula IV, a can be an integer from 1 to 15, from 1 to 12, from 1 to 10, from 1 to 8, from 1 to 5, or from 1 to 3, such as 1 or 2. In some forms of Formula IV, Z′ can be a hydrogen, a substituted or unsubstituted alkyl, a substituted or unsubstituted alkenyl, or a substituted or unsubstituted alkynyl. In some forms of Formula IV, R₄ can be a substituted or unsubstituted alkyl, a substituted or unsubstituted carbonyl, a substituted or unsubstituted alkoxy, an amido, an amino, or a sulfinyl, a sulfonyl, a phosphonium, a phosphanyl, a phosphoryl, a phosphonyl, a thiol, or a sugar group (such as a glucose group or an acetylated glucose). In some forms of Formula IV, R₄ can contain at least one oxygen and can be attached to the carbon through the oxygen.

In some forms of Formula IV, a can be an integer from 1 to 5, from 1 to 3, such as 1 or 2; Z′ can be a hydrogen, a substituted or unsubstituted alkyl, a substituted or unsubstituted alkenyl, or a substituted or unsubstituted alkynyl; and R₄ can be a substituted or unsubstituted alkyl, a substituted or unsubstituted carbonyl, a substituted or unsubstituted alkoxy, an amido, an amino, or a sulfinyl, a sulfonyl, a phosphonium, a phosphanyl, a phosphoryl, a phosphonyl, a thiol, or a sugar group (such as a glucose group or an acetylated glucose).

In some forms, the compound can have the structure of Formula V.

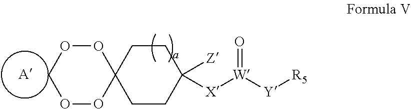

(a) where A′, a, and Z′ can be as described above; (b) where X′ can be

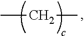

O, NR₆, or S, and c can be an integer from 0 to 30; (c) where W′ can be C, PR₇, S, or Si; (d) where Y′ can be NR₈ or O; (e) where R₆ and R₇ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl; and (f) where R₅ and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl, or R₅ and R₈ taken together with the Y′ to which they are attached form a substituted or unsubstituted cycloalkyl, a substituted or unsubstituted cycloalkenyl, a substituted or unsubstituted cycloalkynyl, a substituted or unsubstituted heterocyclyl, a substituted or unsubstituted aryl, a substituted or unsubstituted heteroaryl, a substituted or unsubstituted polyaryl, or a substituted or unsubstituted polyheteroaryl; where the substituents can be any substituents described above.

In some forms of Formula V, a can be an integer from 1 to 15, from 1 to 12, from 1 to 10, from 1 to 8, from 1 to 5, or from 1 to 3, such as 1 or 2. In some forms of Formula V, Z′ can be hydrogen, a substituted or unsubstituted alkyl, a substituted or unsubstituted alkenyl, or a substituted or unsubstituted alkynyl. In some forms of Formula V, X′ can be

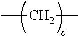

or O, and c can be zero or an integer from 1 to 10, from 1 to 8, from 1 to 5, or from 1 to 3, such as 1 or 2. In some forms of Formula V, X′ can be O. In some forms of Formula V, W′ can be C, PR₇, or S, where R₇ can be a hydrogen, a substituted or unsubstituted alkyl, a substituted or unsubstituted alkenyl, a substituted or unsubstituted alkynyl, an amino, a thiol, or a substituted or unsubstituted alkoxy.

In some forms of Formula V, a can be an integer from 1 to 5, from 1 to 3, such as 1 or 2; Z′ can be a hydrogen, a substituted or unsubstituted alkyl, a substituted or unsubstituted alkenyl, or a substituted or unsubstituted alkynyl; X′ can be

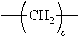

or O, and c can be zero or an integer from 1 to 10, from 1 to 8, from 1 to 5, or from 1 to 3; W′ can be C, PR₇, or S, where R₇ can be a hydrogen, a substituted or unsubstituted alkyl, a substituted or unsubstituted alkenyl, a substituted or unsubstituted alkynyl, an amino, a thiol, or a substituted or unsubstituted alkoxy; Y′ can be NR₈ or O; and R₅ and R₈ can be independently a hydrogen, a substituted or unsubstituted alkyl, a substituted or unsubstituted alkenyl, a substituted or unsubstituted alkynyl, a substituted or unsubstituted aryl, a substituted or unsubstituted aralkyl (such as a substituted or unsubstituted alkylaryl, a substituted or unsubstituted arylalkyl, a substituted or unsubstituted alkylheteroaryl, a substituted or unsubstituted heteroarylalkyl, etc.), a substituted or unsubstituted carbonyl, a substituted or unsubstituted alkoxy, an amido, an amino, or a sulfinyl, a sulfonyl, a phosphonium, a phosphanyl, a phosphoryl, a phosphonyl, a thiol, a silyl, or a sugar group (such as a glucose group or an acetylated glucose), or R₅ and R₈ taken together with Y′ to which they are attached form a substituted or unsubstituted heterocycloalkyl, a substituted or unsubstituted heterocycloalkenyl, or a substituted or unsubstituted heterocycloalkynyl.

In some forms, the compound can have the structure of Formula VI.

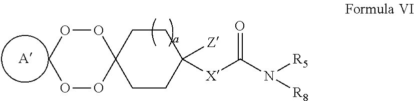

(a) where A′ and a can be as described above; (b) where Z′ can be a hydrogen, substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a phosphonium, or a sugar group (such as a glucose group or an acetylated glucose); (c) where X′ can be

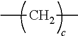

or O, and c can be an integer from 0 to 10; (d) where R₅ and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, or R₅ and R₈ taken together with the nitrogen to which they are attached form a substituted or unsubstituted cycloalkyl, a substituted or unsubstituted cycloalkenyl, a substituted or unsubstituted cycloalkynyl, a substituted or unsubstituted heterocycloalkyl, a substituted or unsubstituted heterocycloalkenyl, a substituted or unsubstituted heterocycloalkynyl, a substituted or unsubstituted aryl, a substituted or unsubstituted heteroaryl, a substituted or unsubstituted polyaryl, or a substituted or unsubstituted polyheteroaryl.

In some forms of Formula VI, X′ can be O. In some forms of Formula VI, X′ can be

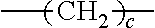

and c can be zero or an integer from 1 to 10, from 1 to 8, from 1 to 5, or from 1 to 3, such as 1 or 2. In some forms of Formula VI, a can be an integer from 1 to 15, from 1 to 12, from 1 to 10, from 1 to 8, from 1 to 5, or from 1 to 3, such as 1 or 2. In some forms of Formula VI, Z′ can be a hydrogen, a substituted or unsubstituted alkyl, a substituted or unsubstituted alkenyl, or a substituted or unsubstituted alkynyl.

In some forms of Formula VI, a can be an integer from 1 to 5, from 1 to 3, such as 1 or 2; Z′ can be a hydrogen, a substituted or unsubstituted alkyl, a substituted or unsubstituted alkenyl, or a substituted or unsubstituted alkynyl; X′ can be O; and R₅ and R₈ can be independently a hydrogen, a substituted or unsubstituted alkyl, a substituted or unsubstituted alkenyl, a substituted or unsubstituted alkynyl, a substituted or unsubstituted aryl, a substituted or unsubstituted aralkyl (such as a substituted or unsubstituted alkylaryl, a substituted or unsubstituted arylalkyl, a substituted or unsubstituted alkylheteroaryl, a substituted or unsubstituted heteroarylalkyl, etc.), a substituted or unsubstituted carbonyl, a substituted or unsubstituted alkoxy, an amido, an amino, or a sulfinyl, a sulfonyl, a phosphonium, a phosphanyl, a phosphoryl, a phosphonyl, a thiol, a silyl, or a sugar group (such as a glucose group or an acetylated glucose), or R₅ and R₈ taken together with the nitrogen to which they are attached form a substituted or unsubstituted heterocycloalkyl, a substituted or unsubstituted heterocycloalkenyl, or a substituted or unsubstituted heterocycloalkynyl.

In some forms of Formulae I-VI, A′ can be a substituted or unsubstituted cycloalkyl, a substituted or unsubstituted cycloalkenyl, a substituted or unsubstituted cycloalkynyl, a substituted or unsubstituted heterocycloalkyl, a substituted or unsubstituted heterocycloalkenyl, a substituted or unsubstituted heterocycloalkynyl, a substituted or unsubstituted aryl, a substituted or unsubstituted heteroaryl, a substituted or unsubstituted polyaryl, a substituted or unsubstituted polyheteroaryl. In some forms of Formulae I-VI, A′ can be a substituted or unsubstituted cycloalkenyl, a substituted or unsubstituted cycloalkynyl, a substituted or unsubstituted heterocycloalkyl, a substituted or unsubstituted heterocycloalkenyl, a substituted or unsubstituted heterocycloalkynyl.

In some forms of Formulae I-VI, A′ can be a substituted or unsubstituted cycloalkyl or a substituted or unsubstituted heterocycloalkyl, such as a substituted or unsubstituted C₃-C₃₀ cycloalkyl, a substituted or unsubstituted C₃-C₃₀ heterocycloalkyl, a substituted or unsubstituted C₃-C₂₅ cycloalkyl, a substituted or unsubstituted C₃-C₂₅ heterocycloalkyl, a substituted or unsubstituted C₃-C₂₀ cycloalkyl, a substituted or unsubstituted C₃-C₂₀ heterocycloalkyl, a substituted or unsubstituted C₃-C₁₅ cycloalkyl, a substituted or unsubstituted C₃-C₁₅ heterocycloalkyl, a substituted or unsubstituted C₃-C₁₀ cycloalkyl, a substituted or unsubstituted C₃-C₁₀ heterocycloalkyl, a substituted or unsubstituted C₅-C₃₀ cycloalkyl, a substituted or unsubstituted C₅-C₃₀ heterocycloalkyl, a substituted or unsubstituted C₅-C₂₅ cycloalkyl, a substituted or unsubstituted C₅-C₂₅ heterocycloalkyl, a substituted or unsubstituted C₅-C₂₀ cycloalkyl, a substituted or unsubstituted C₅-C₂₀ heterocycloalkyl, a substituted or unsubstituted C₅-C₁₅ cycloalkyl, a substituted or unsubstituted C₅-C₁₅ heterocycloalkyl, a substituted or unsubstituted C₅-C₁₀ cycloalkyl, a substituted or unsubstituted C₅-C₁₀ heterocycloalkyl.

In some forms of Formulae I-VI, A′ can be a substituted or unsubstituted C₆-C₃₀ polycycloalkyl, a substituted or unsubstituted C₆-C₃₀ polyheterocycloalkyl, a substituted or unsubstituted C₆-C₂₅ polycycloalkyl, a substituted or unsubstituted C₆-C₂₅ polyheterocycloalkyl, a substituted or unsubstituted C₆-C₂₀ polycycloalkyl, a substituted or unsubstituted C₆-C₂₀ polyheterocycloalkyl, a substituted or unsubstituted C₆-C₁₅ polycycloalkyl, a substituted or unsubstituted C₆-C₁₅ polyheterocycloalkyl, a substituted or unsubstituted C₆-C₁₀ polycycloalkyl, a substituted or unsubstituted C₆-C₁₀ polyheterocycloalkyl, a substituted or unsubstituted C₁₀-C₃₀ polycycloalkyl, a substituted or unsubstituted C₁₀-C₃₀ polyheterocycloalkyl, a substituted or unsubstituted C₁₀-C₂₅ polycycloalkyl, a substituted or unsubstituted C₁₀-C₂₅ polyheterocycloalkyl, a substituted or unsubstituted C₁₀-C₂₀ polycycloalkyl, a substituted or unsubstituted C₁₀-C₂₀ polyheterocycloalkyl, a substituted or unsubstituted C₁₀-C₁₅ polycycloalkyl, a substituted or unsubstituted C₁₀-C₁₅ polyheterocycloalkyl, such as a substituted or unsubstituted C₁₀ polycycloalkyl or a substituted or unsubstituted C₁₀ polyheterocycloalkyl.

In some forms of Formulae I-VI, A′ can be a substituted or unsubstituted C₃-C₁₀ monocycloalkyl, a substituted or unsubstituted C₃-C₁₀ monoheterocycloalkyl, a substituted or unsubstituted C₃-C₅ monocycloalkyl, a substituted or unsubstituted C₃-C₅ monoheterocycloalkyl, a substituted or unsubstituted C₃-C₆ monocycloalkyl, a substituted or unsubstituted C₃-C₆ monoheterocycloalkyl, a substituted or unsubstituted C₃-C₅ monocycloalkyl, a substituted or unsubstituted C₃-C₅ monoheterocycloalkyl, such as a cyclohexyl.

In some forms of Formulae I-VI, A′ can be a substituted adamantylidine, or a substituted or unsubstituted cyclohexyl. In some forms of Formulae I-VI, A′ can be a substituted adamantylidine. In some forms of Formulae I-VI, A′ can be a substituted or unsubstituted cyclohexyl.

In some forms, the compound can have the structure of Formula VII.

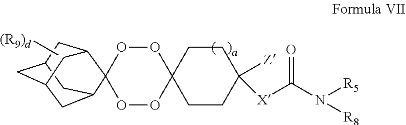

(a) where a, X′, R₅, R₈ can be as described above; (b) where Z′ can be a hydrogen or a substituted or unsubstituted alkyl; and (c) where R₉ can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, and d can be an integer from 0 to 9; where the substituents can be any substituents described above.

In some forms of Formula VII, a can be an integer from 1 to 5, from 1 to 3, such as 1 or 2. In some forms of Formula VII, X′ can be

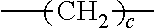

or O, and c can be zero or an integer from 1 to 10, from 1 to 8, from 1 to 5, or from 1 to 3. In some forms of Formula VII, X′ can be O. In some forms of Formula VII, d can be zero. In some forms of Formula VII, d can be an integer from 1 to 5, from 1 to 3, or 1 or 2 and R₉ can be a hydrogen, a substituted or unsubstituted alkyl, a substituted or unsubstituted alkenyl, a substituted or unsubstituted alkynyl, a substituted or unsubstituted aralkyl, a substituted or unsubstituted carbonyl, a substituted or unsubstituted alkoxy, or an amido.

In some forms of Formula VII, a can be an integer from 1 to 5, from 1 to 3, or 1 or 2; X′ can be O; R₅ and R₈ can be independently a hydrogen, a substituted or unsubstituted alkyl, a substituted or unsubstituted aryl, a substituted or unsubstituted heteroaryl, a substituted or unsubstituted aralkyl, a substituted or unsubstituted carbonyl, a substituted or unsubstituted alkoxy, an amido, an amino, a phosphonium, a phosphanyl, or a silyl, or R₅ and R₈ taken together with the nitrogen to which they are attached form a substituted or unsubstituted cycloalkyl, a substituted or unsubstituted cycloalkenyl, a substituted or unsubstituted cycloalkynyl, a substituted or unsubstituted heterocycloalkyl, a substituted or unsubstituted heterocycloalkenyl, or a substituted or unsubstituted heterocycloalkynyl; and R₉ is a substituted or unsubstituted alkyl, a substituted or unsubstituted alkenyl, a substituted or unsubstituted alkynyl, a substituted or unsubstituted carbonyl, or a substituted or unsubstituted alkoxy.

In some forms, the compound can have the structure of Formula VIII.

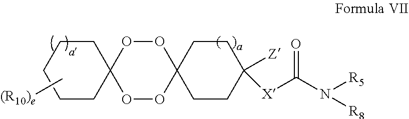

(a) where a, X′, R₅, and R₈ can be as described above; (b) where a′ can be an integer from 1 to 20; (c) where Z′ can be a hydrogen or a substituted or unsubstituted alkyl; and (d) where R₁₀ can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amido, an amino, a phosphonium, a phosphanyl, or a silyl, and e can be an integer from 0 to 24; where the substitutes can be any substituents described above.

In some forms of Formula VIII, a and a′ can be each an integer from 1 to 5, from 1 to 3, such as 1 or 2. In some forms of Formula VIII, X′ can be

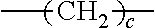

or O, and c can be zero or an integer from 1 to 10, from 1 to 8, from 1 to 5, or from 1 to 3. In some forms of Formula VIII, X′ can be O.

In some forms of Formula VIII, e can be zero. In some forms of Formula VIII, e can be an integer from 1 to 5, from 1 to 4, or 1 or 2 and where R₁₀ can be a substituted or unsubstituted alkyl, a substituted or unsubstituted alkenyl, a substituted or unsubstituted alkynyl, a substituted or unsubstituted carbonyl, or a substituted or unsubstituted alkoxy. In some forms of Formula VIII, e can be an integer from 1 to 5, from 1 to 4, or 1 or 2 and where R₁₀ can be a substituted or unsubstituted branched C₁-C₂₀ alkyl group, a substituted or unsubstituted branched C₁-C₁₅ alkyl group, a substituted or unsubstituted branched C₁-C₁₀ alkyl group, a substituted or unsubstituted branched C₁-C₈ alkyl group, a substituted or unsubstituted branched C₁-C₆ alkyl group, or a substituted or unsubstituted branched C₁-C₄ alkyl group. In some forms of Formula VIII, e can be an integer from 1 to 5, from 1 to 4, or 1 or 2; and where R₁₀ can be

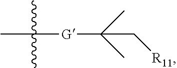

where G′ can be

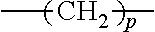

and p can be an integer from 0 to 10, from 0 to 8, from 0 to 6, from 0 to 4, from 0 to 3, or from 0 to 2, and where R₁₁ can be a hydrogen or

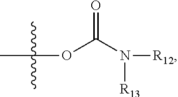

where R₁₂ and R₁₃ can be independently a hydrogen, a substituted or unsubstituted alkyl, a substituted or unsubstituted alkenyl, or a substituted or unsubstituted alkynyl. In some forms of Formula VIII, e can be an integer from 1 to 5, from 1 to 4, or 1 or 2 and R₁₀ can be a substituted or unsubstituted tert-butyl, where the substituents can be any substituents described above, such as a substituted or unsubstituted alkenyl, a substituted or unsubstituted alkynyl, a substituted or unsubstituted carbonyl, a substituted or unsubstituted alkoxyl, or an amido.

In some forms, the compound can have the structure of Formula IX.

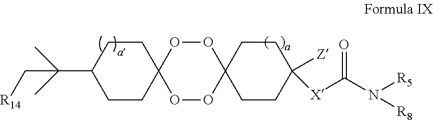

(a) where a, a′, Z, X′, R₅, and R₈ can be as described above; and (b) where R₁₄ can be a hydrogen, a substituted or unsubstituted alkenyl, a substituted or unsubstituted alkynyl, a substituted or unsubstituted carbonyl, a substituted or unsubstituted alkoxyl, or an amido.

In some forms of Formula IX, a and a′ can be each an integer from 1 to 5, from 1 to 3, such as 1 or 2. In some forms of Formula IX, X′ can be

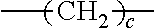

or O, and c can be zero or an integer from 1 to 10, from 1 to 8, from 1 to 5, or from 1 to 3. In some forms of Formula IX, X′ can be O. In some forms of IX, R₁₄ can be a hydrogen or

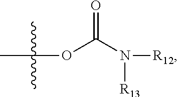

where R₁₂ and R₁₃ can be independently a hydrogen, a substituted or unsubstituted alkyl, a substituted or unsubstituted alkenyl, or a substituted or unsubstituted alkynyl.

In some forms of Formulae V-IX, R₅ and R₈ can be independently a hydrogen, a substituted or unsubstituted alkyl, a substituted or unsubstituted alkenyl, a substituted or unsubstituted alkynyl, a substituted or unsubstituted aryl, a substituted or unsubstituted aralkyl (such as a substituted or unsubstituted alkylaryl, a substituted or unsubstituted arylalkyl, a substituted or unsubstituted alkylheteroaryl, a substituted or unsubstituted heteroarylalkyl, etc.), a substituted or unsubstituted carbonyl, a substituted or unsubstituted alkoxy, an amido, an amino, or a sulfinyl, a sulfonyl, a phosphonium, a phosphanyl, a phosphoryl, a phosphonyl, a thiol, a silyl, or a sugar group (such as a glucose group or an acetylated glucose), or R₅ and R₈ taken together with the nitrogen to which they are attached form a substituted or unsubstituted heterocycloalkyl, a substituted or unsubstituted heterocycloalkenyl, or a substituted or unsubstituted heterocycloalkynyl. In some forms of Formulae V-IX, R₅ and R₈ can be a phosphonium, an amino, or a silyl.

In some forms of Formula IX, R₅ and R₈ can be independently

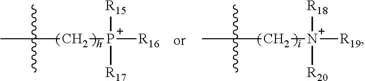

where h and i can be independently an integer from 0 to 10, from 0 to 8, from 0 to 6, or from 0 to 3, where R₁₅-R₂₀ can be independently a hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, a substituted or unsubstituted alkylaryl, or a substituted or unsubstituted arylalkyl, such as a methyl, an ethyl, a propanyl, a butyl, a pentyl, a hexyl, a phenyl, or a benzyl.

In some forms of Formulae I-IX, the substituents for a substituted functional group can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a phosphonium, a phosphanyl, a phosphoryl, a phosphonyl, or a sugar group (such as a glucose group or an acetylated glucose), or a combination thereof.

In some forms of Formulae I-IX, the substituents for a substituted functional group can be an unsubstituted alkyl, an unsubstituted alkenyl, an unsubstituted alkynyl, an unsubstituted heterocyclyl, an unsubstituted aryl, an unsubstituted heteroaryl, an unsubstituted aralkyl, an unsubstituted carbonyl, an unsubstituted alkoxy, an amino, an amido, a phosphonium, or a sugar group (such as a glucose group or an acetylated glucose), or a combination thereof.

The compound can have the structure of Formula X.

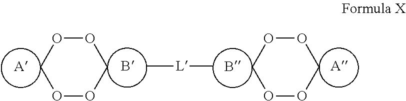

(a) where A′ and A″ can be independently a substituted or unsubstituted monocyclic ring, a substituted or unsubstituted polycyclic ring; (b) where L′ can be a linker, such as an oxygen, a sulfur, a carbon, a boron, a substituted or unsubstituted alkoxy, a substituted or unsubstituted alkyl, a substituted or unsubstituted alkenyl, a substituted or unsubstituted cycloalkyl, a substituted or unsubstituted cycloalkenyl, a substituted or unsubstituted aryl, a substituted or unsubstituted heterocycloalkyl, a substituted or unsubstituted heteroaryl, a phosphoryl, a sulfinyl, a sulfonyl, an ether, a polyether, a disulfide, and an amino; and (c) where the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group (such as a glucose group or an acetylated glucose), or a combination thereof.

In some forms, A′ and A″ can be the same. In some forms, A′ and A″ can be different. In some forms, B′ and B″ can be the same. In some forms, B′ and B″ can be different. In some forms, A′ and A″ can be the same and B′ and B″ can be different. In some forms, A′ and A″ can be different and B′ and B″ can be the same. In some forms, A′ and A″ can be different and B′ and B″ can be different. In some forms, A′ and A″ can be the same and B′ and B″ can be the same. When A′ and A″ are the same and B′ and B″ are the same, the compound of Formula X is a dimer.

In some forms, the compound can have the structure of Formula XI.

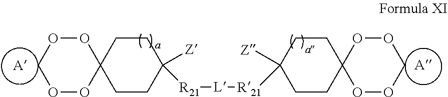

(a) where A′, A″, and L′ can be as described above; (b) where a and a″ can be independently an integer from 1 to 20, from 1 to 15, from 1 to 10, from 1 to 5, from 1 to 3, or 1 or 2; (c) where Z′, Z″, R₂₁, and R′₂₁ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a silyl, a sulfinyl, a sulfonyl, a phosphonium, a phosphanyl, a phosphoryl, a phosphonyl, a thiol, or a sugar group (such as a glucose group or an acetylated glucose); where the substituents can be any substituents described above.

In some forms, the compound of Formula XI can be a dimer, where A′ and A″ are the same, Z and Z″ are the same, and R₂₁ and R′₂₁ are the same.

In some forms, the compound can have the structure of Formula XII.

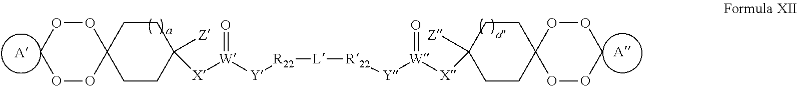

(a) where A′, A″, L′, a, a″, Z′, and Z″ can be as described above; (b) where X′ and X″ can be independently

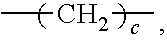

O, NR₆, or S, and c can be an integer from 0 to 30, from 0 to 25, from 0 to 20, from 0 to 15, from 0 to 10, from 0 to 5, from 0 to 3, or 0, 1, or 2; (c) where W′ and W″ can be independently C, PR₇, S, or Si; (d) where Y′ and Y″ can be independently a bond or is NR₈ or O; (e) where R₂₂ and R′₂₂ can be independently

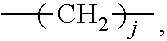

and j is an integer from 0 to 20, from 0 to 15, from 0 to 10, from 0 to 5, from 0 to 3, or 0, 1, or 2; and (f) where R₆, R₇, and R₈ can be independently a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an aroxy, an amido, an amino, a phosphonium, a phosphanyl, a phosphonyl, phosphoryl, a phosphate, a silyl, a sulfinyl, a thiol, or a hydroxyl; where the substituents can be any substituents described above.

In some forms of Formulae XI and XII, Z′ and Z″ can be independently a hydrogen or a substituted or unsubstituted alkyl. In some forms of Formula XII, X′ and X″ can be independently

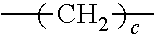

or O, and c can be an integer from 0 to 10, from 0 to 5, from 0 to 3, or 0, 1, or 2. In some forms of Formula XII, W′ and W″ can be independently C, PR₇, or S, where R₇ can be R₇ can be a hydrogen, a substituted or unsubstituted alkyl, a substituted or unsubstituted alkenyl, a substituted or unsubstituted alkynyl, an amino, a thiol, or a substituted or unsubstituted alkoxy. In some forms, the compound of Formula XII can be a dimer, where A′ and A″ are the same, Z and Z″ are the same, X′ and X″ are the same, Y′ and Y″ are the same, and R₂₁ and R′₂₁ are the same.

In some forms of Formulae X-XII, L′ can be a phosphoryl, a sulfinyl, a sulfonyl, a disulfide, an ether, a polyether.

In some forms of Formulae X-XII, the substituents for a substituted functional group can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, an amino, an amido, a phosphonium, a phosphanyl, a phosphoryl, a phosphonyl, or a sugar group (such as a glucose group or an acetylated glucose), or a combination thereof.

In some forms of Formulae X-XII, the substituents for a substituted functional group can be an unsubstituted alkyl, an unsubstituted alkenyl, an unsubstituted alkynyl, an unsubstituted heterocyclyl, an unsubstituted aryl, an unsubstituted heteroaryl, an unsubstituted aralkyl, an unsubstituted carbonyl, an unsubstituted alkoxy, an amino, an amido, a phosphonium, or a sugar group (such as a glucose group or an acetylated glucose), or a combination thereof.

The compounds may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The compounds may be capable of existing as geometric isomers. Accordingly, it is to be understood that the present invention includes pure geometric isomers or mixtures of geometric isomers.

### 2. Exemplary 1,2,4,5-Tetraoxanes Derivatives

Exemplary 1,2,4,5-Tetraoxanes derivatives are presented below.

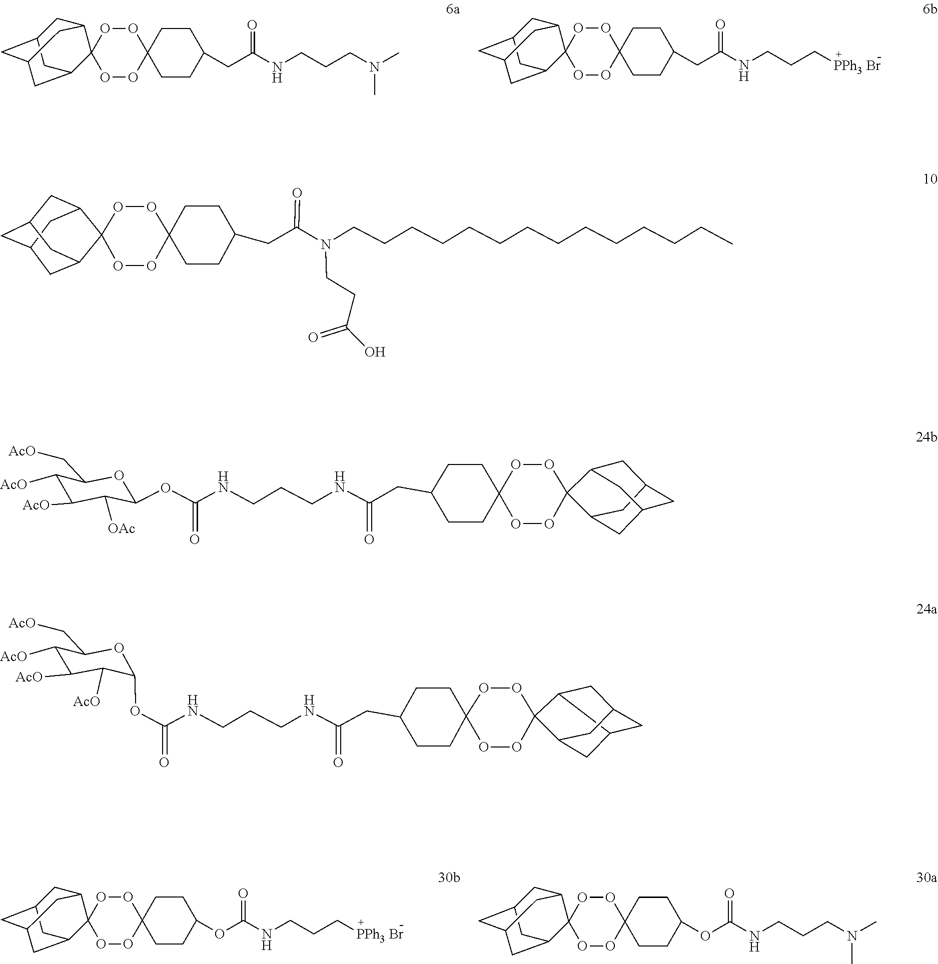

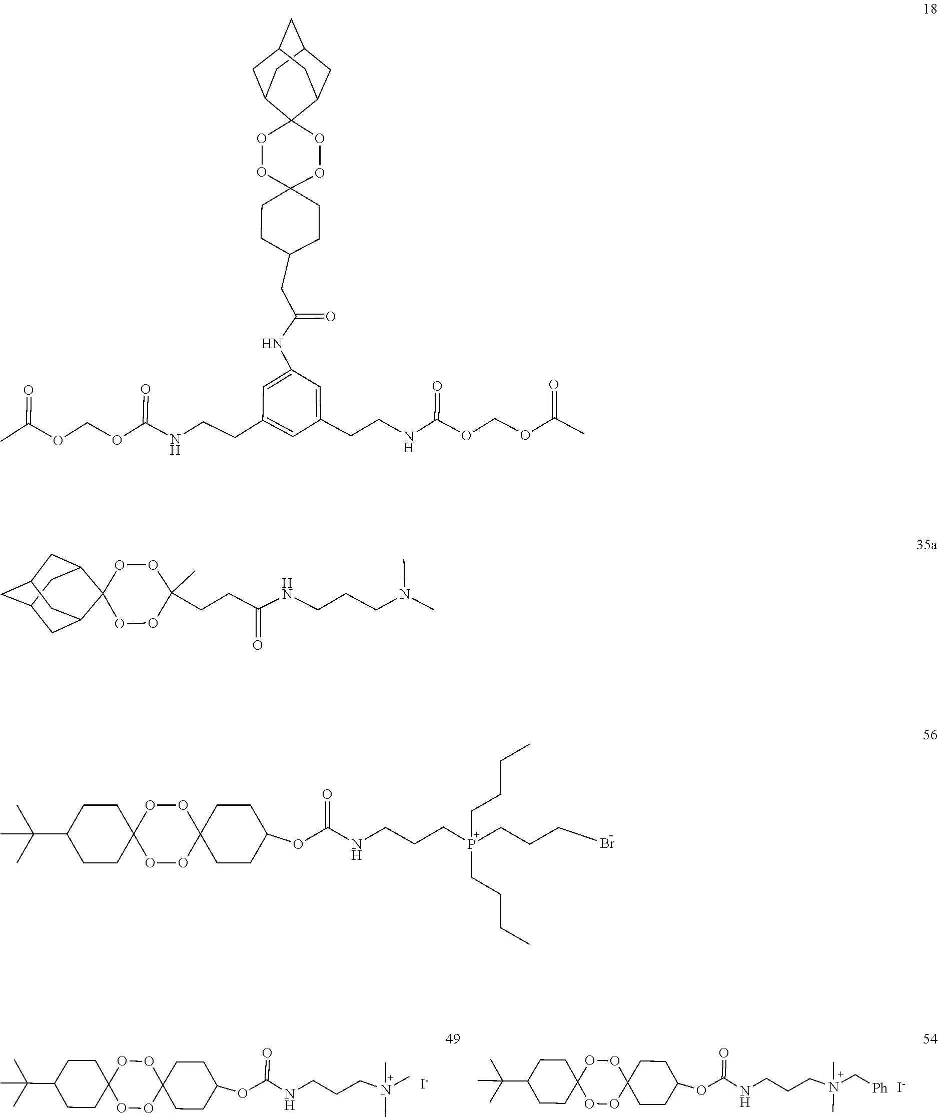

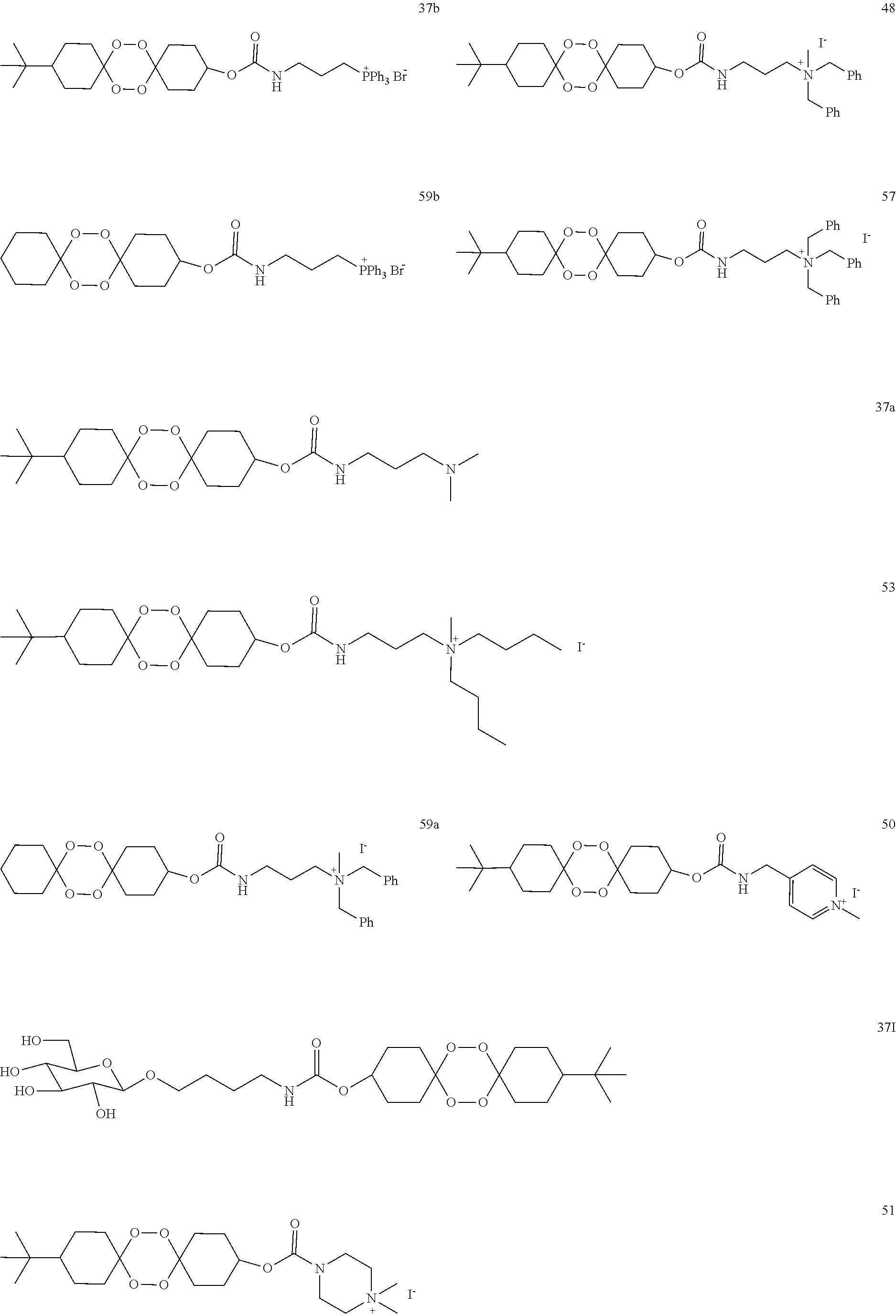

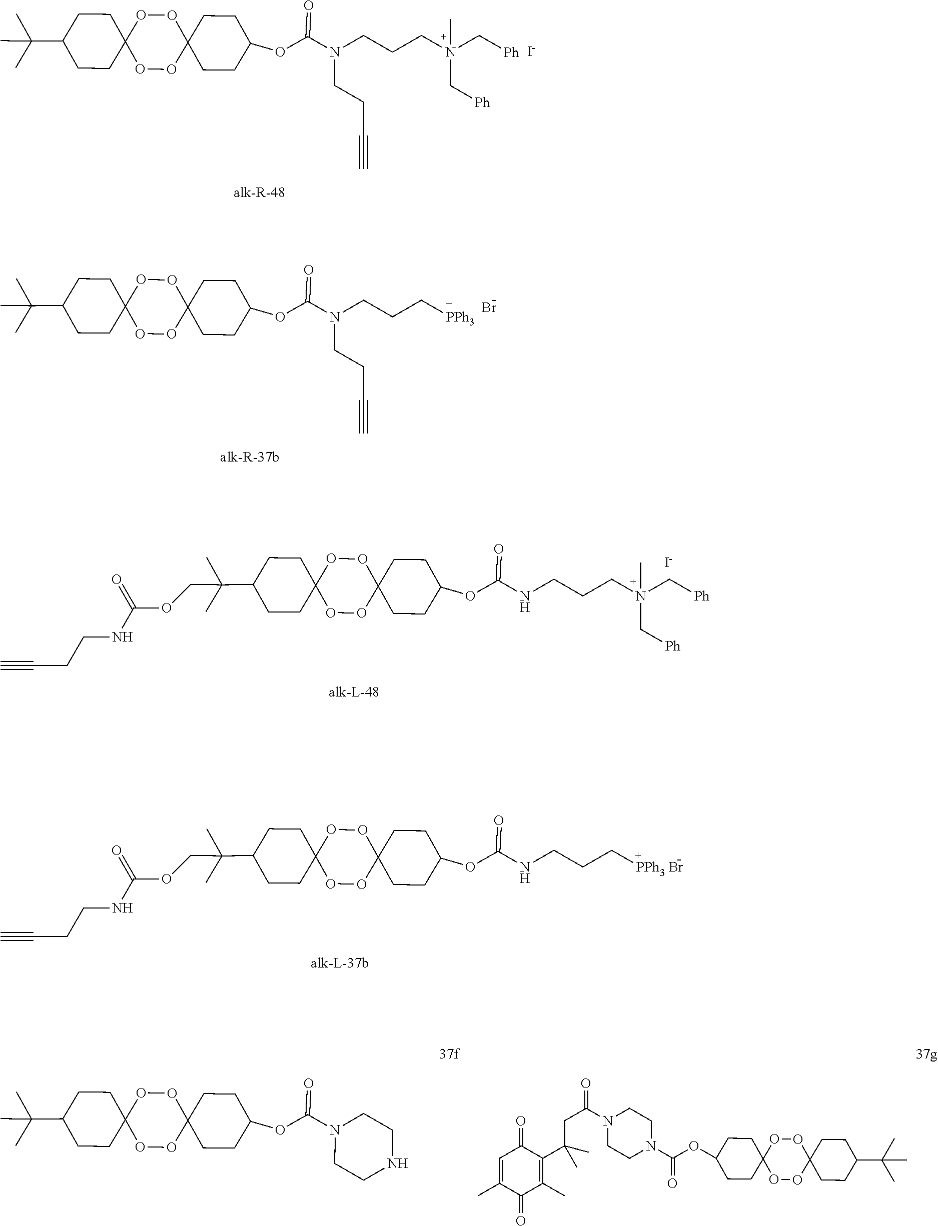

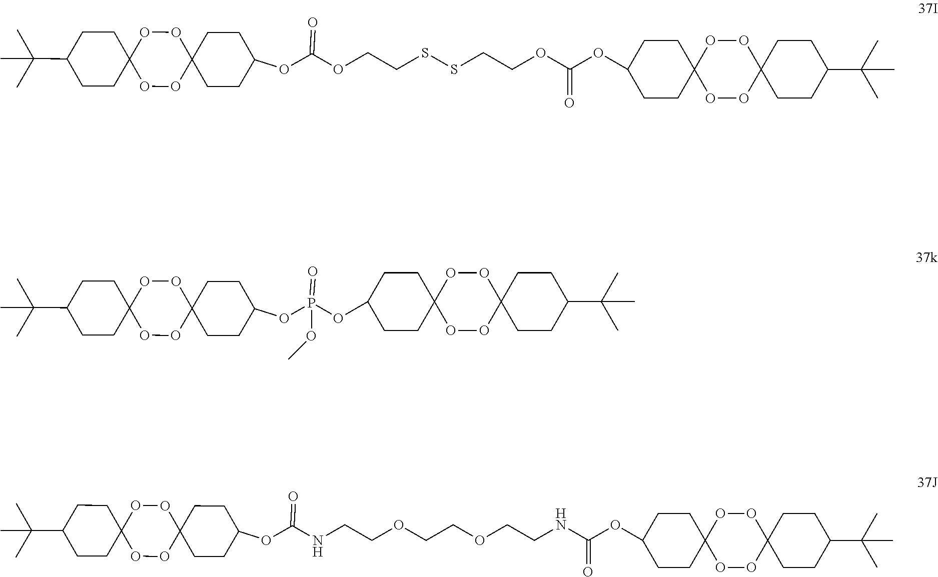

### 3. Pharmaceutically Acceptable Salts

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

B. Pharmaceutical Compositions

Pharmaceutical compositions and pharmaceutical formulations in unit dosage form (also referred herein as “pharmaceutical formulations”) suitable for the delivery of the compounds thereof and their preparation are disclosed. Generally, the pharmaceutical composition or formulation contains the compounds and/or the pharmaceutically acceptable salts of the compounds described herein, and a pharmaceutically acceptable excipient. The term “pharmaceutically acceptable excipient” is used herein to describe any ingredient in the formulation other than the compounds described herein. The pharmaceutical compositions or formulations can include an effective amount of one or more compounds of any of the formulae described herein and/or their pharmaceutically acceptable salts, including any one or any combination of compounds of the formulae described herein and/or their pharmaceutically acceptable salts, for treating a cancer, reducing a cancer, or treating or ameliorating one or more symptoms associated with a cancer in a subject in need thereof. In some forms, the pharmaceutical compositions or formulations can contain enzymes that catalyze hydrogen peroxide production and/or iron oxide. In some forms, the pharmaceutical compositions or formulations do not contain enzymes that catalyze hydrogen peroxide production and/or iron oxide.

In some forms, the pharmaceutical composition or formulation can further contain one or more active agents in addition to the compounds, such as one or more additional anticancer agents.

It is to be understood that combinations and/or mixtures of the compounds and/or their pharmaceutically acceptable salts may be included in the composition or formulation. In some forms, the pharmaceutical composition or formulation includes an effective amount of the compounds and/or their pharmaceutically acceptable salts for treating a cancer, reducing a cancer, or treating or ameliorating one or more symptoms associated with a cancer in a subject in need thereof.

Any one of more of the compounds provided herein can be expressly included or expressly excluded from the pharmaceutical compositions, dosage units, and/or methods of use or treatment disclosed herein.

1. Oral Formulations

The compounds and/or their pharmaceutically acceptable salts can be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The compounds and/or their pharmaceutically acceptable salts may also be used in fast-dissolving, fast-disintegrating dosage forms such as those described in Expert Opinion in Therapeutic Patents, 11 (6), 981-986, by Liang and Chen (2001).

For tablet or capsule dosage forms, depending on dose, the compounds and/or their pharmaceutically acceptable salts may make up from 1 weight % to 80 weight % of the dosage form, more typically from 5 weight % to 60 weight % of the dosage form. In addition to the compounds described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), anti-oxidants, colourants, flavouring agents, preservatives and taste-masking agents.

Exemplary tablets contain up to about 80% of one or more of the compounds described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations for oral administration may be formulated to be immediate and/or modified release. Modified release formulations include delayed, sustained, pulsed, controlled, targeted and programmed release formulations.

2. Parenteral Formulations

The compounds and/or their pharmaceutically acceptable salts can also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the compounds used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

3. Pulmonary and Mucosal Formulations

The compounds and/or their pharmaceutically acceptable salts can be formulated for pulmonary or mucosal administration. The administration can include delivery of the composition to the lungs, nasal, oral (sublingual, buccal), vaginal, or rectal mucosa.

For example, the compounds can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff”. The overall daily dose will be administered in a single dose or, more usually, as divided doses throughout the day.

In some forms, the compounds and/or their pharmaceutically acceptable salts can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical compositions may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

4. Topical Formulations

The compounds and/or their pharmaceutically acceptable salts can be administered directly to the external surface of the skin or the mucous membranes (including the surface membranes of the nose, lungs and mouth), such that the compounds and/or their pharmaceutically acceptable salts cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

5. Additional Active Agent(s)

In some forms, the pharmaceutical composition or pharmaceutical formulation can include one or more additional active agents, such as one or more additional anticancer agents. Anticancer agents that can be included in the pharmaceutical compositions or formulations are known, for example, see the National Cancer Institute database, “A to Z List of Cancer Drugs,” website cancer.gov/about-cancer/treatment/drugs.

Exemplary anticancer drugs that can be included in the pharmaceutical composition or pharmaceutical formulation include, but are not limited to olaparib, abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

6. Effective Amount

Effective amount of the compounds contained in the pharmaceutical composition or pharmaceutical formulation depend on many factors, including the indication being treated, the route of administration, co-administration of other therapeutic compositions, and the overall condition of the patient. Exemplary effective amount of the compounds contained in the pharmaceutical formulation (in unit dosage form) can be from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg.

### III. Methods of Making

The compounds can be synthesized using methods known in the art of organic synthesis, such as methods that use a starting material or more than one starting materials in a suitable solvent medium to form tetraoxanes; then the formed tetraoxanes are derived with a targeting group. Typically, the starting material that can be used to form tetraoxanes is a ketone or an acetyl. Exemplary ketones and acetals forming the tetraoxanes include, but are not limited to, 4-tert-butylcyclohexanone, cyclohexanone, 2-adamantanone, and starting materials 1, 3, and 26 shown below. Typically, the targeting group contains an amine, such as N, N′-dimethylpropylamine, 3-bromopropylamine hydrobromide, and targeting group 68 shown below.

For example, as shown in the general reaction scheme below, starting materials 1, 3, or 26, or a combination thereof reacts with 4-tert-butylcyclohexanone, cyclohexanone, or 2-adamantanone, or a combination thereof to form tetraoxanes; the formed tetraoxanes then react with N, N′-dimethylpropylamine, 3-bromopropylamine hydrobromide, or targeting group 68, or a combination thereof to form the compounds disclosed herein.

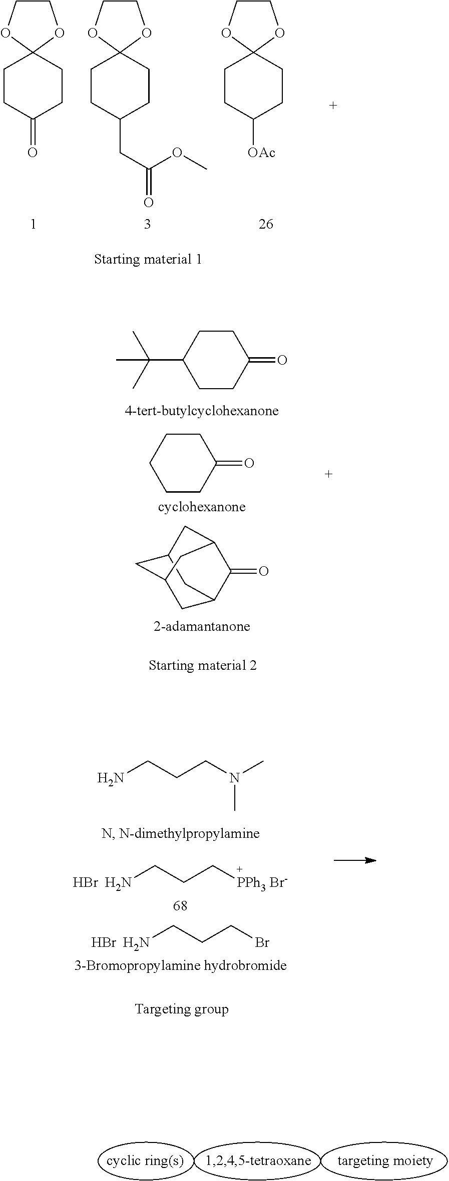

Additional exemplary starting materials and targeting groups, and more specific methods for synthesizing exemplary compounds, are described in the Examples below.

### IV. Methods of Use

A. Treating Cancer, Reducing Cancer, or Treating or Ameliorating Symptom(s) Associated with Cancer

Methods of using the compounds for treating a cancer, reducing a cancer, or treating or ameliorating one or more symptoms associated with a cancer in a subject in need thereof are disclosed.

Generally, the method includes (i) administering to the subject an effective amount of the compound(s) to treat the cancer, reduce the cancer, or treat or ameliorate one or more symptoms associated with the cancer in the subject. The subject can be a mammal. In some forms, the subject can be at risk of, exhibiting symptoms of, or diagnosed with cancer. The compound(s) can be administered by a medical professional or the subject being treated (e.g. self-administration).

In some forms of the method, whether cancer is reduced may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as HER2 for breast cancer, PSA for prostate cancer, or others.

In some forms, the compounds and/or their pharmaceutically acceptable salts can be administered in the form of a pharmaceutical composition or formulation in association with one or more pharmaceutically acceptable excipients, such as the pharmaceutical composition or formulation described above. The choice of the pharmaceutically acceptable excipients will to a large extent depend on factors such as the particular mode of administration, the effect of the excipient on solubility and stability, and the nature of the dosage form.

1. Cancers

As used herein, the term “cancer” refers to any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area.

In some forms of the method, the cancer can be tumors, such as tumors of the hematopoietic and lymphoid tissues or hematopoietic and lymphoid malignancies, tumors that affect the blood, bone marrow, lymph, and lymphatic system, and tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus.

In some forms of the method, the cancer can be a colon cancer, breast cancer, ovarian cancer, cervical cancer, lung cancer, rectal cancer, kidney cancer, liver cancer, brain cancer, or leukemia, or a combination thereof. In some forms of the method, the cancer can be breast cancer, such as triple negative breast cancer (TNBC).

In some forms of the method, the cancer can be AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

2. Administration Routes

The compound(s) and/or their pharmaceutically acceptable salts or pharmaceutical composition or formulation containing the compound(s) and/or their pharmaceutically acceptable salts can be administered to the subject by oral administration, parenteral administration, inhalation, mucosal, topical administration, or a combination thereof.

For example, the compound(s) and/or their pharmaceutically acceptable salts or the pharmaceutical composition or formulation containing the compound(s) and/or their pharmaceutical acceptable slats can be orally administered to a subject by a medical professional or the subject being treated (e.g. self-administration). The compound(s) or the pharmaceutical composition or formulation containing the compound(s) and/or their pharmaceutical acceptable slats can be administered as tablets, capsules containing particulates, granules, powders, lozenges (including liquid-filled lozenges), chews, multi- and nano-particulates, gels, or liquids (e.g. solution or suspensions in aqueous or non-aqueous solvent).

Optionally, the compound(s) and/or their pharmaceutically acceptable salts or the pharmaceutical composition or formulation containing the compound(s) and/or their pharmaceutical acceptable slats can be administered to the subject by intravenous injection or intraperitoneal injection. The intravenous injection or intraperitoneal injection can be performed by a medical professional or the subject being treated (e.g. self-injection).

Alternatively, the compound(s) and/or their pharmaceutically acceptable salts or the pharmaceutical composition or formulation containing the compound(s) and/or their pharmaceutical acceptable slats can be administered to the subject by inhalation, such as mouth inhalation and/or nasal inhalation.

Optionally, the compound(s) and/or their pharmaceutically acceptable salts or the pharmaceutical composition or formulation containing the compound(s) and/or their pharmaceutical acceptable slats can be administered to the subject by topically applying the compound(s) or the pharmaceutical composition or formulation on one or more of the exposed surfaces of the subject.

3. Effective Amount

The therapeutically effective amount of the compounds depend on many factors, including the indication being treated, the route of administration, co-administration of other therapeutic compositions, and the overall condition of the patient.

In general, treatment regimens utilizing compounds include administration of from about 0.1 mg to about 300 mg of the compounds per kilogram body weight of the recipient per day in multiple doses or in a single dose. In some embodiments, a suitable dose may be in the range of 0.1 to 300 mg per kilogram body weight of the recipient per day, optionally in the range of 6 to 150 mg per kilogram body weight per day, optionally in the range of 15 to 100 mg per kilogram body weight per day, optionally in the range of 15 to 80 mg per kilogram body weight per day, optionally in the range of 15 to 50 mg per kilogram body weight per day, and optionally in the range of 15 to 30 mg per kilogram body weight per day.

The desired dose may be presented as two, three, four, five or six or more sub-doses administered at appropriate intervals throughout the day. These sub-doses may be administered in unit dosage forms, for example, containing from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg of the compounds per unit dosage form.

4. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some forms, the one or more additional active agents is included in a pharmaceutical composition or formulation containing the compound(s) and is administered to the subject simultaneously with the compound(s) in the pharmaceutical composition or formulation in association with one or more pharmaceutically acceptable excipients.

In some forms, the one or more additional active agents are one or more anticancer agents described above. The amount of the one or more additional anticancer agents required will vary from subject to subject according to their need.

B. Treating Cancer Cells

In some forms, the compounds can be used in a method for treating cancer cells and/or cancer stem cells in a subject in need thereof.

The method can follow the method step described above, for example, administering to the subject an effective amount of the compound by oral administration, parenteral administration, inhalation, mucosal, topical administration, or a combination thereof. In some forms, the method can include the additional step described above. For example, the user can administer one or more additional active agents to the subject prior to, during, and/or subsequent to administering the compound to the subject.

In some forms of the method, the compounds can trigger ferroptosis to kill the cancer cells and/or the cancer stem cells of the cancer in the subject. Additionally or alternatively, the compounds can selectively trigger ferroptosis in cancer cells and cancer stem cells compared with non-cancerous cells in the subject. Additionally or alternatively, the compounds can generate reactive oxygen species inside the cancer cells and cancer stem cells regardless of the pH (e.g., generate hydroxyl radicals and lipid peroxides under neutral pH), eliminating complex formulations that encapsulate multiple components (e.g., enzymes that catalyze hydrogen peroxide production, iron oxide, etc.) and the requirement of acidic intracellular environment in chemodynamic therapy, i.e. ferroptosis. For example, the compound can induce ferroptosis in the cancer cells and/or cancer stem cells where the intracellular pH of the cancer cells and/or cancer stem cells is in a range from 6 to 7.5.

In some forms of the method, the compound can have an IC₅₀ value against the cancer cells or cancer stem cells lower than an IC₅₀ value of the same compound against non-cancerous cells, tested under the same condition. Alternatively or additionally, the compound can have an IC₅₀ value against the cancer cells or cancer stem cells lower than an IC₅₀ value of a known compound, such as OZ277, OZ439, RKA182, OZ277, OZ439, RKA182, FINO₂, or a cholic acid/deoxycholic acid/steroid derivative of 1,2,4,5-tetraoxane, against the same cancer cells or cancer stem cells, tested under the same condition.

1. Cancer Cell Lines

The cancer cells and/or cancer stem cells being treated in the subject can be the cancer cells of any one of the cancers described above. For example, the cancer cells can be MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof. In some forms of the method, the cancer cells or cancer stem cells can be MDA-MB-231 cells, HCT116 cells, HEYA8 cells, or HL-60 cells, or a combination thereof. In some forms of the method, the cancer cells or cancer stem cells are MDA-MB-231 cells.

When comparing the IC₅₀ values of the compound against cancer cells or cancer stem cells with the IC₅₀ values of the same compound against non-cancerous cells, the non-cancerous cells can be from any normal tissue of the subject, such as NIH3T3 cells, MDCK cells, or bEnd.3 cells, or a combination thereof.

2. Compound Selectivity Against Cancer Cells Over Non-Cancerous Cells

In some forms of the method, the compound can have an IC₅₀ value against the cancer cells or cancer stem cells lower than an IC₅₀ value of the same compound against non-cancerous cells, tested under the same condition. The term “same conditions” means test is performed using the same assay, such as MTT assay, using the same protocol, such as same amount of cells and enzymes, same dye and dye concentration, and same incubation time and temperature, etc.

For example, the compound can have an IC₅₀ value against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof lower than an IC₅₀ value of the same compound against NIH3T3 cells, MDCK cells, or bEnd.3 cells, or a combination thereof, such as NIH3T3 cells, tested under the same condition. For example, the compound can have an IC₅₀ value against MDA-MB-231 cells, HCT116 cells, or HL-60 cells lower than an IC₅₀ value of the same compound against NIH3T3 cells, tested under the same condition. For example, the compound can have an IC₅₀ value against MDA-MB-231 cells lower than an IC₅₀ value of the same compound against NIH3T3 cells, tested under the same condition.

In some forms, the IC₅₀ value of the compound against the cancer cells or cancer stem cells is at least 2 times lower, at least 3 times lower, at least 4.5 times lower, at least 5 times lower, at least 8 times lower, at least 10 times lower, at least 12 times lower, at least 15 times lower, at least 20 times lower, at least 22 times lower, at least 24 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 90 times lower, at least 100 times lower, in a range from 2 to 1000 times lower, in a range from 2 to 500 times lower, in a range from 2 to 250 times lower, in a range from 2 to 200 times lower, in a range from 2 to 150 times lower, in a range from 2 to 100 times lower, in a range from 5 to 1000 times lower, in a range from 5 to 500 times lower, in a range from 5 to 250 times lower, in a range from 5 to 200 times lower, in a range from 5 to 150 times lower, or in a range from 5 to 100 times lower than an IC₅₀ value of the same compound against non-cancerous cells, tested under the same condition.

For example, the compound can have an IC₅₀ value against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof that is at least 2 times lower, at least 3 times lower, at least 4.5 times lower, at least 5 times lower, at least 8 times lower, at least 10 times lower, at least 12 times lower, at least 15 times lower, at least 20 times lower, at least 22 times lower, at least 24 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 90 times lower, at least 100 times lower, in a range from 2 to 1000 times lower, in a range from 2 to 500 times lower, in a range from 2 to 250 times lower, in a range from 2 to 200 times lower, in a range from 2 to 150 times lower, in a range from 2 to 100 times lower, in a range from 5 to 1000 times lower, in a range from 5 to 500 times lower, in a range from 5 to 250 times lower, in a range from 5 to 200 times lower, in a range from 5 to 150 times lower, or in a range from 5 to 100 times lower than an IC₅₀ value of the same compound against NIH3T3 cells, MDCK cells, or bEnd.3 cells, or a combination thereof, tested under the same condition.

For example, the compound can have an IC₅₀ value against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof that is at least 2 times lower, at least 3 times lower, at least 4.5 times lower, at least 5 times lower, at least 8 times lower, at least 10 times lower, at least 12 times lower, at least 15 times lower, at least 20 times lower, at least 22 times lower, at least 24 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 90 times lower, at least 100 times lower, in a range from 2 to 1000 times lower, in a range from 2 to 500 times lower, in a range from 2 to 250 times lower, in a range from 2 to 200 times lower, in a range from 2 to 150 times lower, in a range from 2 to 100 times lower, in a range from 5 to 1000 times lower, in a range from 5 to 500 times lower, in a range from 5 to 250 times lower, in a range from 5 to 200 times lower, in a range from 5 to 150 times lower, or in a range from 5 to 100 times lower than an IC₅₀ value of the same compound against NIH3T3 cells, tested under the same condition.

For example, the compound can have an IC₅₀ value against MDA-MB-231 cells, HCT116 cells, or HL-60 cells at least 2 times lower, at least 3 times lower, at least 4.5 times lower, at least 5 times lower, at least 8 times lower, at least 10 times lower, at least 12 times lower, at least 15 times lower, at least 20 times lower, at least 22 times lower, at least 24 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 90 times lower, at least 100 times lower, in a range from 2 to 1000 times lower, in a range from 2 to 500 times lower, in a range from 2 to 250 times lower, in a range from 2 to 200 times lower, in a range from 2 to 150 times lower, in a range from 2 to 100 times lower, in a range from 5 to 1000 times lower, in a range from 5 to 500 times lower, in a range from 5 to 250 times lower, in a range from 5 to 200 times lower, in a range from 5 to 150 times lower, or in a range from 5 to 100 times lower than an IC₅₀ value of the same compound against NIH3T3 cells, tested under the same condition.

For example, the compound can have an IC₅₀ value against MDA-MB-231 cells at least 2 times lower, at least 3 times lower, at least 4.5 times lower, at least 5 times lower, at least 8 times lower, at least 10 times lower, at least 12 times lower, at least 15 times lower, at least 20 times lower, at least 22 times lower, at least 24 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 90 times lower, at least 100 times lower, in a range from 2 to 1000 times lower, in a range from 2 to 500 times lower, in a range from 2 to 250 times lower, in a range from 2 to 200 times lower, in a range from 2 to 150 times lower, in a range from 2 to 100 times lower, in a range from 5 to 1000 times lower, in a range from 5 to 500 times lower, in a range from 5 to 250 times lower, in a range from 5 to 200 times lower, in a range from 5 to 150 times lower, or in a range from 5 to 100 times lower than an IC₅₀ value of the same compound against NIH3T3 cells, tested under the same condition.

Exemplary IC₅₀ values of exemplary compounds against cancer cells and cancer stem cells, such as against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells and against non-cancerous cells, such as NIH3T3 cells, MDCK cells, and bEnd.3 cells, tested under specific conditions are described in the Examples below.

3. Compound Cytotoxicity against Cancer Cells Compared with Other Compounds

In some forms of the method, the compound can have an IC₅₀ value against the cancer cells or cancer stem cells lower than an IC₅₀ value of a known compound, such as OZ277, OZ439, RKA182, FINO₂, or a cholic acid/deoxycholic acid/steroid derivative of 1,2,4,5-tetraoxane, against the same cancer cells or cancer stem cells, tested under the same condition.

For example, the compound can have an IC₅₀ value against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof lower than an IC₅₀ value of a known compound, such as OZ277, OZ439, RKA182, FINO₂, or a cholic acid/deoxycholic acid/steroid derivative of 1,2,4,5-tetraoxane, against the same cancer cells, tested under the same condition. For example, the compound can have an IC₅₀ value against MDA-MB-231 cells, HCT116 cells, or HL-60 cells lower than an IC₅₀ value of a known compound, such as OZ277, OZ439, RKA182, FINO₂, or a cholic acid/deoxycholic acid/steroid derivative of 1,2,4,5-tetraoxane, against the same cancer cells, tested under the same condition. For example, the compound can have an IC₅₀ value against MDA-MB-231 cells lower than an IC₅₀ value of a known compound, such as OZ277, OZ439, RKA182, FINO₂, or a cholic acid/deoxycholic acid/steroid derivative of 1,2,4,5-tetraoxane, against the same cancer cells, tested under the same condition. OZ277, OZ439, RKA182, FINO₂, and cholic acid/deoxycholic acid/steroid derivative of 1,2,4,5-tetraoxanes are described in O'Neill P. M., et al., Angew Chem Int Ed Engl, 2010, 49(33), 5693-5697; Opsenica D., et al., Bioorg Med Chem., 2003, 3; 11(13):2761-8; Coghi P., et al., ChemMedChem., 2018, 13(9):902-908; Amewu R. K., et al., Bioorg Med Chem., 2013, 21(23), 7392-7397; Marti F., et al., MedChemComm., 2011, 2(7); Terzic N., et al., J Med Chem., 2007, 50(21), 5118-5127; Opsenica D., et al., J Med Chem., 2000, 43(17), 3274-3282; Solaja B. A., et al., J Med Chem., 202, 45(16), 3331-3336; Opsenica D., et al., Bioorg Med Chem., 2003, 11(13), 2761-2768; Opsenica D., et al., J. Serb. Chem. Soc., 2015, 80 (11) 1339-1359; Dong Y. X., et al., J. Org. Chem., 1998, 63, 23, 8582-8585; Abrams R. P., et al., ACS Chem Biol, 2016, 11(5), 1305-1312; Zhang Y., et al., Cell Chem Biol., 2019, 26(5):623-633; Friedmann Angeli J. P., et al., Nat Cell Biol., 2014, 16(12):1180-91; Llabani E., et al., J. Nat Chem., 2019, 11(6):521-532; Dolma S., et al., Cancer Cell, 2003, 3(3), 285-296; Mai T. T., et al., Nat Chem, 2017, 9(10), 1025-1033; WO 2008038030; WO 2010134678; IN 2008DE02103; U.S. Pat. No. 6,906,098; DE 10205864; DE10205864; WO 9307119; and WO2010109172.

In some forms, the IC₅₀ value of the compound against the cancer cells or cancer stem cells is at least 5 times lower, at least 10 times lower, at least 15 times lower, at least 20 times lower, at least 25 times lower, at least 30 times lower, at least 35 times lower, in a range from 5 to 1000 times lower, in a range from 5 to 500 times lower, in a range from 5 to 250 times lower, in a range from 5 to 200 times lower, in a range from 5 to 150 times lower, in a range from 5 to 100 times lower, in a range from 10 to 1000 times lower, in a range from 10 to 500 times lower, in a range from 10 to 250 times lower, in a range from 10 to 200 times lower, in a range from 10 to 150 times lower, from 15 to 1000 times lower, in a range from 15 to 500 times lower, in a range from 15 to 250 times lower, in a range from 15 to 200 times lower, in a range from 15 to 150 times lower, from 20 to 1000 times lower, in a range from 20 to 500 times lower, in a range from 20 to 250 times lower, in a range from 20 to 200 times lower, in a range from 20 to 150 times lower, from 25 to 1000 times lower, in a range from 25 to 500 times lower, in a range from 25 to 250 times lower, in a range from 25 to 200 times lower, in a range from 25 to 150 times lower, from 30 to 1000 times lower, in a range from 30 to 500 times lower, in a range from 30 to 250 times lower, in a range from 30 to 200 times lower, or in a range from 30 to 150 times lower than an IC₅₀ value of a known compound against the same cancer cells, tested under the same condition.

For example, the compound can have an IC₅₀ value against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, or HL-60 cells, or a combination thereof that is at least 5 times lower, at least 10 times lower, at least 15 times lower, at least 20 times lower, at least 25 times lower, at least 30 times lower, at least 35 times lower, in a range from 5 to 1000 times lower, in a range from 5 to 500 times lower, in a range from 5 to 250 times lower, in a range from 5 to 200 times lower, in a range from 5 to 150 times lower, in a range from 5 to 100 times lower, in a range from 10 to 1000 times lower, in a range from 10 to 500 times lower, in a range from 10 to 250 times lower, in a range from 10 to 200 times lower, in a range from 10 to 150 times lower, from 15 to 1000 times lower, in a range from 15 to 500 times lower, in a range from 15 to 250 times lower, in a range from 15 to 200 times lower, in a range from 15 to 150 times lower, from 20 to 1000 times lower, in a range from 20 to 500 times lower, in a range from 20 to 250 times lower, in a range from 20 to 200 times lower, in a range from 20 to 150 times lower, from 25 to 1000 times lower, in a range from 25 to 500 times lower, in a range from 25 to 250 times lower, in a range from 25 to 200 times lower, in a range from 25 to 150 times lower, from 30 to 1000 times lower, in a range from 30 to 500 times lower, in a range from 30 to 250 times lower, in a range from 30 to 200 times lower, or in a range from 30 to 150 times lower than an IC₅₀ value of OZ277, OZ439, RKA182, FINO₂, or a cholic acid/deoxycholic acid/steroid derivative of 1,2,4,5-tetraoxane against the same cancer cells, tested under the same condition.

For example, the compound can have an IC₅₀ value against MDA-MB-231 cells, HCT116 cells, or HL-60 cells at least 5 times lower, at least 10 times lower, at least 15 times lower, at least 20 times lower, at least 25 times lower, at least 30 times lower, at least 35 times lower, in a range from 5 to 1000 times lower, in a range from 5 to 500 times lower, in a range from 5 to 250 times lower, in a range from 5 to 200 times lower, in a range from 5 to 150 times lower, in a range from 5 to 100 times lower, in a range from 10 to 1000 times lower, in a range from 10 to 500 times lower, in a range from 10 to 250 times lower, in a range from 10 to 200 times lower, in a range from 10 to 150 times lower, from 15 to 1000 times lower, in a range from 15 to 500 times lower, in a range from 15 to 250 times lower, in a range from 15 to 200 times lower, in a range from 15 to 150 times lower, from 20 to 1000 times lower, in a range from 20 to 500 times lower, in a range from 20 to 250 times lower, in a range from 20 to 200 times lower, in a range from 20 to 150 times lower, from 25 to 1000 times lower, in a range from 25 to 500 times lower, in a range from 25 to 250 times lower, in a range from 25 to 200 times lower, in a range from 25 to 150 times lower, from 30 to 1000 times lower, in a range from 30 to 500 times lower, in a range from 30 to 250 times lower, in a range from 30 to 200 times lower, or in a range from 30 to 150 times lower than an IC₅₀ value of a known compound, such as OZ277, OZ439, RKA182, FINO₂, or a cholic acid/deoxycholic acid/steroid derivative of 1,2,4,5-tetraoxane, against the same cancer cells, tested under the same condition.

For example, the compound can have an IC₅₀ value against MDA-MB-231 cells at least 5 times lower, at least 10 times lower, at least 15 times lower, at least 20 times lower, at least 25 times lower, at least 30 times lower, at least 35 times lower, in a range from 5 to 1000 times lower, in a range from 5 to 500 times lower, in a range from 5 to 250 times lower, in a range from 5 to 200 times lower, in a range from 5 to 150 times lower, in a range from 5 to 100 times lower, in a range from 10 to 1000 times lower, in a range from 10 to 500 times lower, in a range from 10 to 250 times lower, in a range from 10 to 200 times lower, in a range from 10 to 150 times lower, from 15 to 1000 times lower, in a range from 15 to 500 times lower, in a range from 15 to 250 times lower, in a range from 15 to 200 times lower, in a range from 15 to 150 times lower, from 20 to 1000 times lower, in a range from 20 to 500 times lower, in a range from 20 to 250 times lower, in a range from 20 to 200 times lower, in a range from 20 to 150 times lower, from 25 to 1000 times lower, in a range from 25 to 500 times lower, in a range from 25 to 250 times lower, in a range from 25 to 200 times lower, in a range from 25 to 150 times lower, from 30 to 1000 times lower, in a range from 30 to 500 times lower, in a range from 30 to 250 times lower, in a range from 30 to 200 times lower, or in a range from 30 to 150 times lower than an IC₅₀ value of a known compound, such as OZ277, OZ439, RKA182, FINO₂, or a cholic acid/deoxycholic acid/steroid derivative of 1,2,4,5-tetraoxane, against the same cancer cells, tested under the same condition.

Exemplary IC₅₀ values of exemplary compounds and exemplary known compounds against cancer cells and cancer stem cells, such as against MDA-MB-231 cells, MCF7 cells, Hela cells, T47D cells, Huh7 cells, PLC cells, U2OS cells, HEK293 cells, HepG2 cells, Jurkat cells, HCT116 cells, HEYA8 cells, and HL-60 cells, and against non-cancerous cells such as NIH3T3 cells, MDCK cells, and bEnd.3 cells, tested under specific conditions are described in the Examples below.

The present invention will be further understood by reference to the following non-limiting examples.

### EXAMPLES

### Example 1. Synthesis of Exemplary Compounds

Compound Structures

Compounds Containing adamantylidine

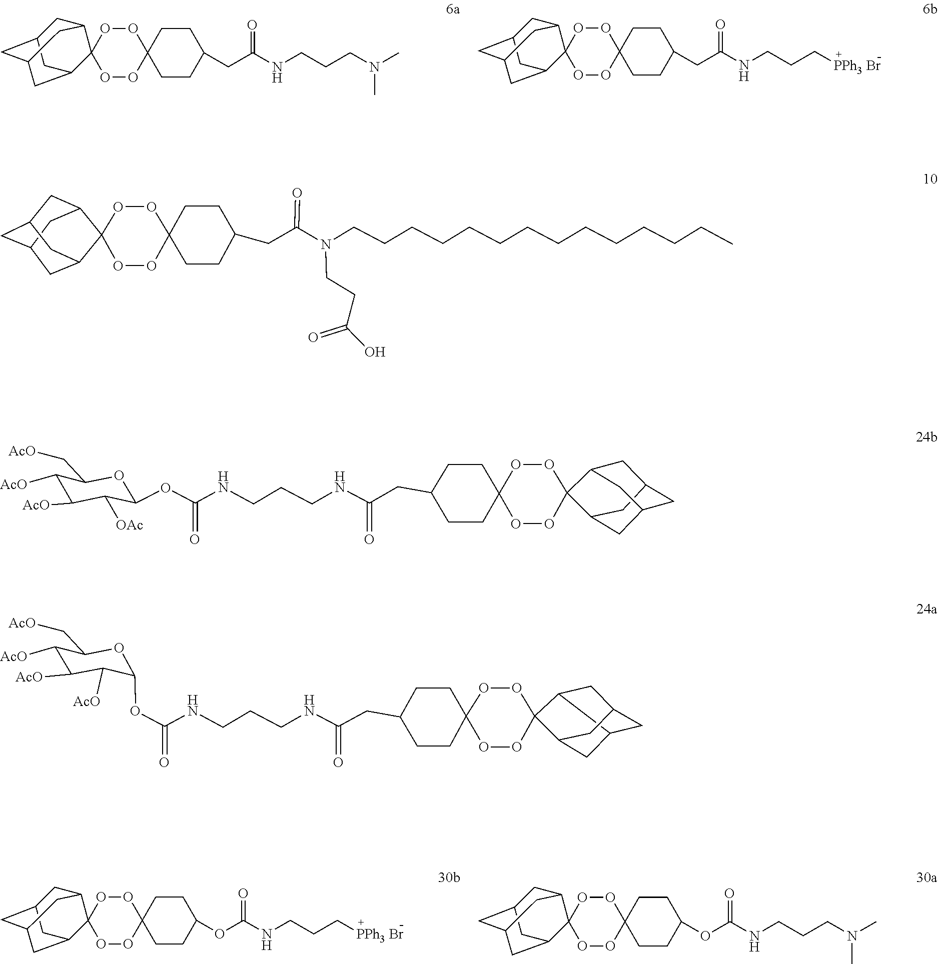

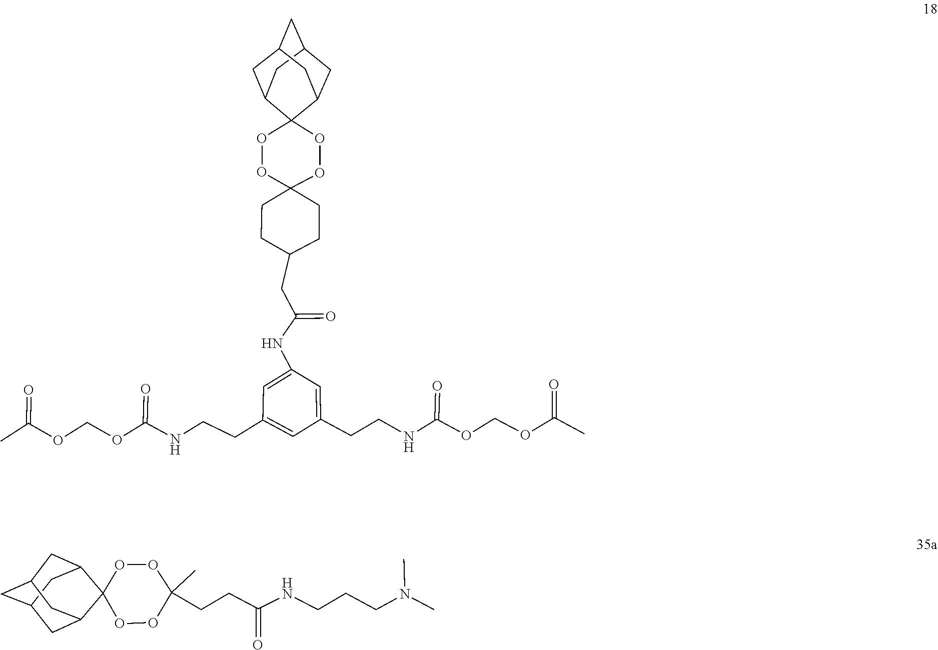 [SMILES: CC(=O)OCOC(=O)NCCc1cc(CCNC(=O)OCOC(C)=O)cc(NC(=O)CC2CCC3(CC2)OOC2(OO3)C3CC4CC(C3)CC2C4)c1 ; CN(C)CCCNC(=O)CCC1(C)OOC2(OO1)C1CC3CC(C1)CC2C3]

Compounds Containing tert-butylcyclohexyl

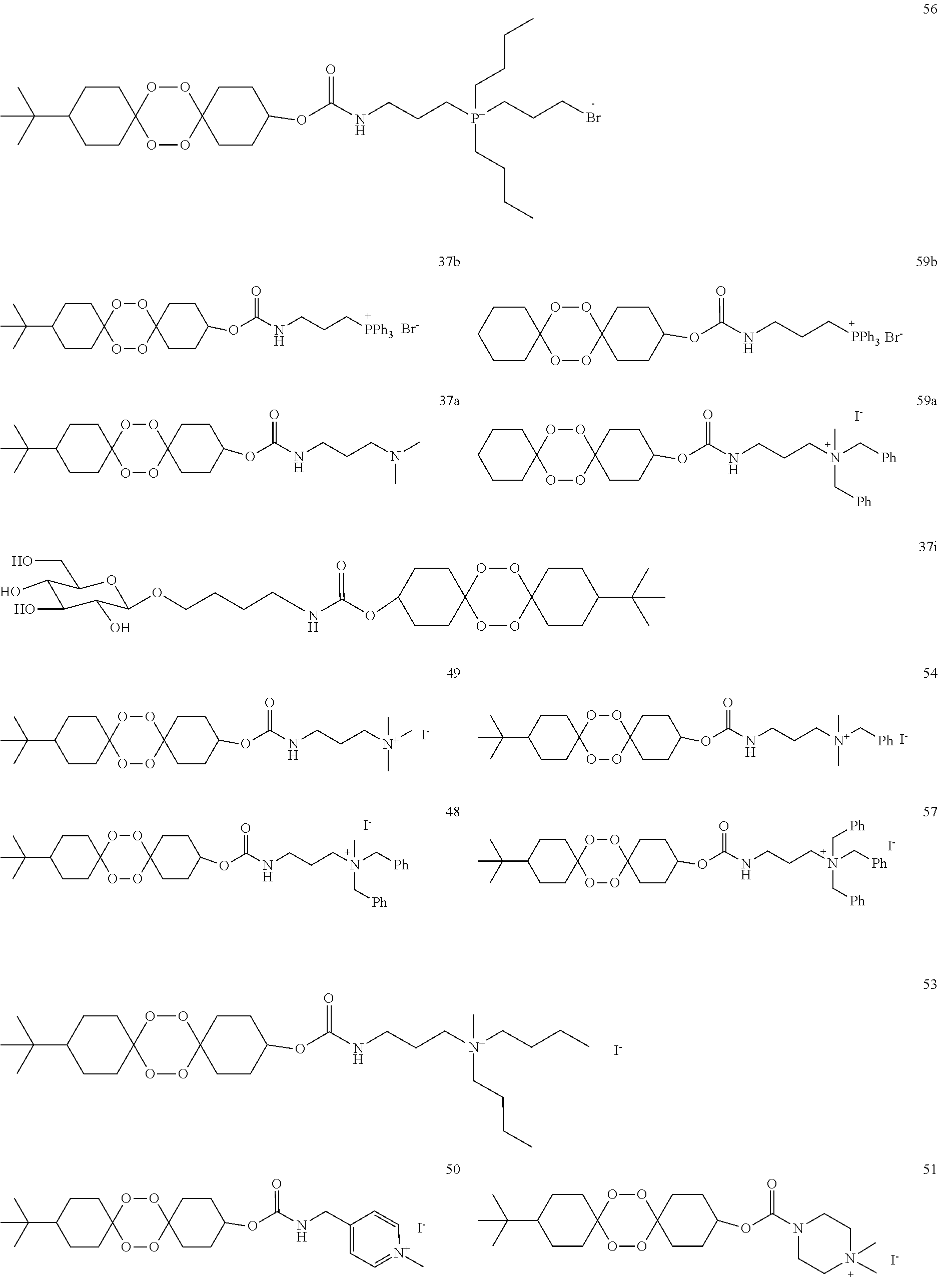

Alkyne-Tagged tetraoxanes

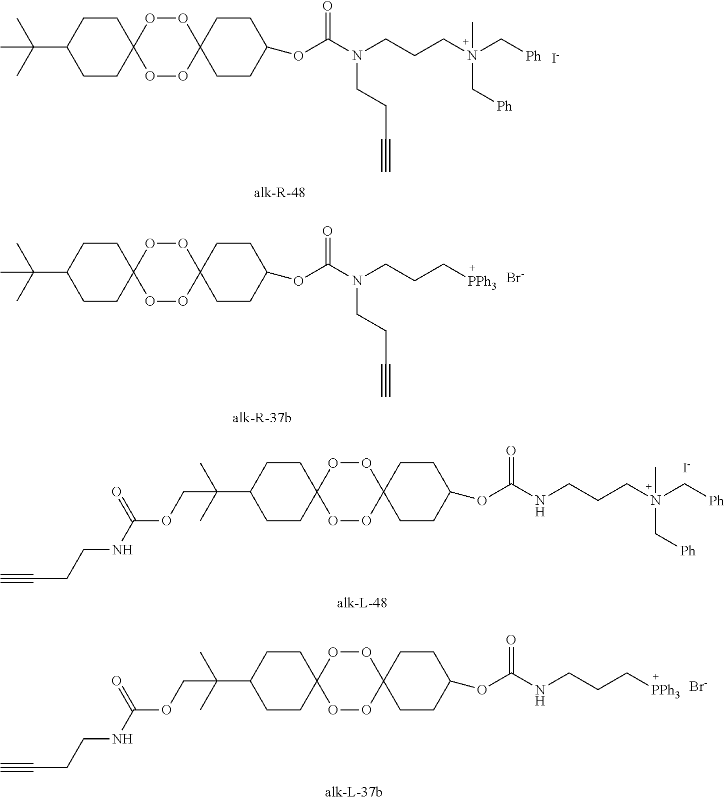

Prodrugs and Dimers

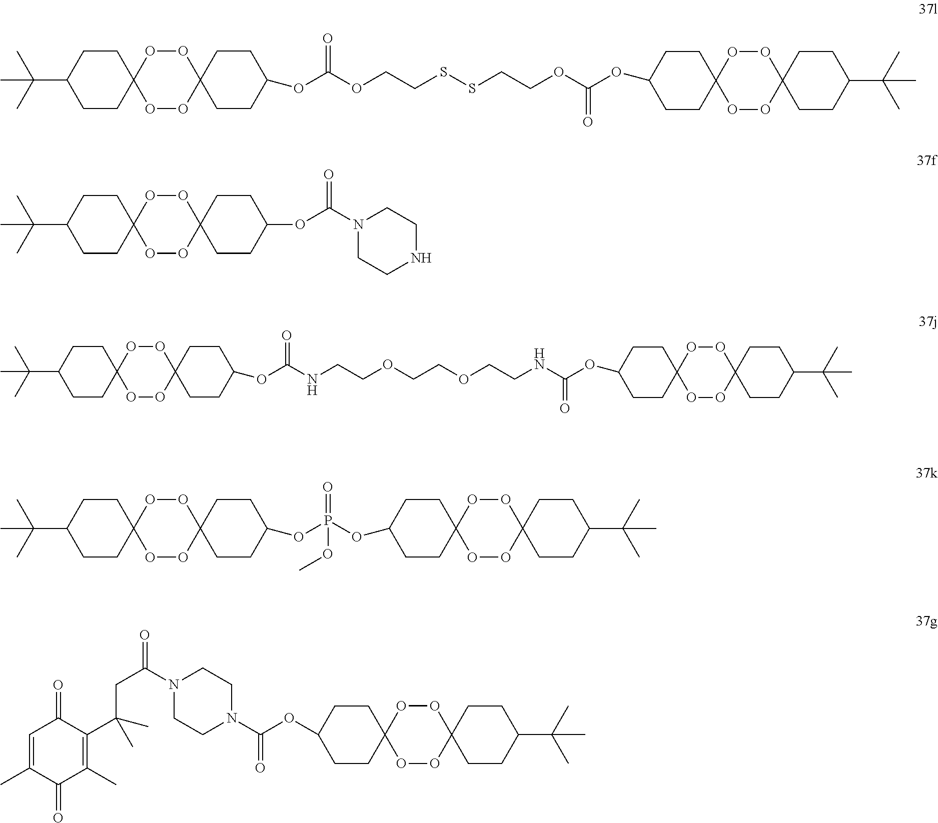

Material and Methods

### Chemical Synthesis

All reagents and solvents for reactions were of analytical or HPLC grade and were dried and distilled if necessary. Analytical thin layer chromatography was performed with silica gel 60 Å F254 plates and spots were visualized by UV and/or staining in phophomolybdic acid (PMA) or KMnO₄ solution followed by heating. Flash chromatography was performed using the indicated solvent system on silica gel (230-400 mesh). Tetrahydrofuran (THF), methylene chloride (CH₂Cl₂), diethyl ether (Et₂O), ethyl acetate (EtOAc) and N, N-dimethylformamide (DMF) were dried by filtration through alumina. All dry reactions were run under an atmosphere of either argon or nitrogen in flame-dried glassware. In general, hydrogen peroxide and organic peroxides should be handled with care. Avoid exposure to strong heat, light, mechanical shock, oxidizable organic materials, and metals. All reactions should be carried out behind a blast shield. Excess hydrogen peroxide was quenched by adding into sodium metabisulphite solution slowly (Liu, et al. Water Res. 2003, 37, 15, 3697-3703; Keen et al. J. Environ Eng. 2013, 139, 137-140).

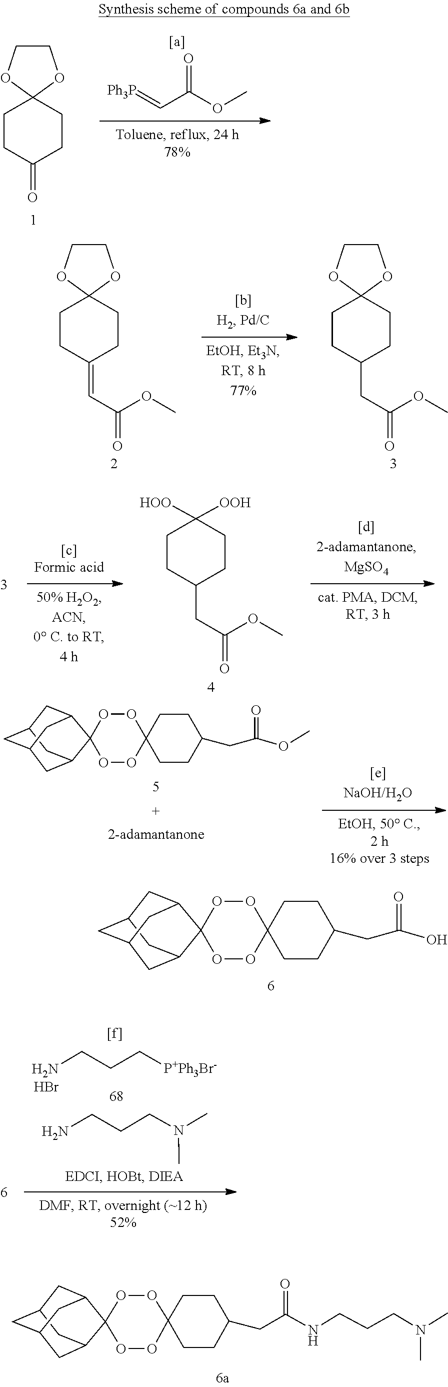

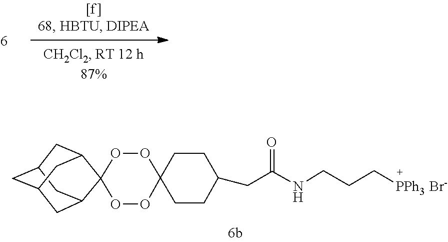

Compound 2 was prepared according to literature report (O'Neill, et al. Angew Chem Int Ed. 2010, 49, 5693-5697; Yan, et al. Synlett. 2011, 2827-2830). To a solution of 1,4-cyclohexanedione monoethylene acetal (1) (5.00 g, 32 mmol) in toluene (76 mL) was added trimethylphosphonoacetate (16.00 g, 48 mmol). The mixture was heated at reflux overnight. Reaction mixture was cooled down to RT, and the solvent was removed under reduced pressure. The crude material was diluted with diethyl ether (70 mL) to precipitate out triphenylphosphine oxide. Insoluble material was filtered through a short pad of celite, and the filtrate was concentrated under reduced pressure. The residue was purified by silica gel column chromatography (n-hexane/EtOAc=4/1) to afford 2 (5.60 g, 78% yield) as colourless oil. 3 was prepared according to literature report (O'Neill, et al. Angew Chem Int Ed. 2010, 49, 5693-5697; Yan, et al. Synlett. 2011, 2827-2830). 2 (6.440 g, 30.3 mmol) was pre-mixed with palladium on charcoal (10% by weight, 3.20 g, 30.3 mmol). Reaction solvent (EtOH, 50 mL) was evacuated and back-filled with hydrogen gas three times before adding into the reaction mixture. Et₃N (0.1 mL, 2.3 mmol) was added. The reaction flask was then evacuated and back-filled with hydrogen gas three times before leaving to stir under an atmosphere of hydrogen (balloon). Reaction was monitored by TLC (n-hexane/EtOAc=4:1). 2 was consumed after 5 h. The reaction mixture was filtered through a pad of Celite, which was then rinsed with EtOH. The filtrate was concentrated under reduced pressure, and the residue was purified by silica gel column chromatography (n-hexane/EtOAc=4/1) to afford 3 (5.011 g, 77% yield) as colourless oil.

Compound 6 were prepared according to literature report with modifications and isolated after purification by flash column chromatography (16% for 3 steps) as white solid (O'Neill, et al. Angew Chem Int Ed. 2010, 49, 5693-5697; Yan, et al. Synlett. 2011, 2827-2830). To a stirred solution of 3 (1.30 g, 7.6 mmol) in formic acid/acetonitrile (1:1, 17 mL) at 0° C. was added 50% aqueous hydrogen peroxide (1.1 mL, 38.2 mmol) and the mixture was allowed to stir at room temperature. TLC analysis (n-Hexane/EtOAc=2:1) indicated that 3 was consumed after 3 h. The mixture was then poured into ice-cold water and extracted with CH₂Cl₂. The combined organic extracts were washed with brine, dried over Na₂SO₄, filtered and concentrated under reduced pressure to give the corresponding gem-dihydroperoxide 4. The solvent CH₂Cl₂ was dried and distilled. Dehydrated PMA was prepared by the procedure; commercially available PMA hydrate was dried in a microwave oven to a constant weight. A mixture of 2-adamantanone (1.26 g, 8.4 mmol), PMA (0.14 g, 1 mol %), and anhydrous MgSO₄ (1.38 g, 11.5 mmol) in CH₂Cl₂ (25 mL) was stirred for 30 min at rt. To this solution was added 4 (7.6 mmol) in CH₂Cl₂ (10 mL) dropwisely in 15 min. The mixture was stirred at rt and monitored by TLC (n-Hexane: EtOAc=4:1). When 4 was consumed completely, deionized H₂O (10 mL) was added. The aqueous layer was extracted with CH₂Cl₂ (3×10 mL). The combined organic phase was washed by brine, dried by Na₂SO₄ and concentrated under reduced pressure. The residue was purified by silica gel column chromatograph (n-hexane/EtOAc=8:1) to afford a mixture of 5 and 2-adamantanone. Isolation of analytically pure 5 by flash chromatography on silica gel was not possible since it co-elutes with 2-adamantanone. Therefore the product mixture was carried forward to the next step. To a solution of 5 and 2-adamantanone (1.308 g total) in EtOH (17 mL) was added a solution of sodium hydroxide (0.45 g, 11.1 mmol) in deionized water (2.7 mL). The mixture was heated at 50° C. for 2 h. Then the solution was allowed to cool to room temperature and concentrated under reduced pressure. The crude was taken in water (20 mL) and washed with diethyl ether (3×30 mL). The aqueous layer was acidified to pH 1 with 1M HCl and then extracted with EtOAc (3×30 mL). The combined organic phases were washed with brine (30 mL), dried over Na₂SO₄, filtered and concentrated under reduced pressure to give the pure compound. Carboxylic acid 6 (0.4199 g, 16% yield for 3 steps) was obtained as a white solid with no further purification required. TPP salt 68 were prepared according to literature report and isolated after recrystallization as a white solid (Millard, et al. J Med Chem. 2013, 56, 22, 9170-9179). To the solution of compound 6 (0.010 g, 0.036 mmol) in CH₂Cl₂ (1 mL) was added DIPEA (0.1 mL) and HBTU (0.015 g, 0.046 mmol). After stirring for 5 min, 68 (0.014 g, 0.036 mmol) was added. The reaction was stirred at room temperature for 12 h. Solvent was removed by reduced pressure. The crude product was purified by silica gel column chromatography (10% EtOH in DCM) to afford compound 6b (0.016 g, 87% yield) as a foam. To a solution of compound 6 (0.017 g, 0.050 mmol) in DMF (1 mL) at 0° C. was added DIPEA (19.1 μL, 0.11 mmol). After 5 min, EDCI (0.017 g, 0.11 mmol), HOBt (0.023 g, 0.11 mmol) and 3-(Dimethylamino)-1-propylamine (0.014 mL, 0.11 mmol) were added subsequently. The reaction mixture was stirred at room temperature for 12 h. The reaction was quenched by NH₄Cl (10 mL) and diluted with EtOAc. The aqueous layer was back-extracted with EtOAc (3×30 mL). The combined organic phases were washed with saturated NaHCO₃, brine (30 mL), dried over Na₂SO₄, filtered and concentrated under reduced pressure to give crude product, which was purified by silica gel column chromatography (7% EtOH in DCM, 1% NH₄OH) to afford compound 6a (0.011 g, 52% yield) as a pink oil.

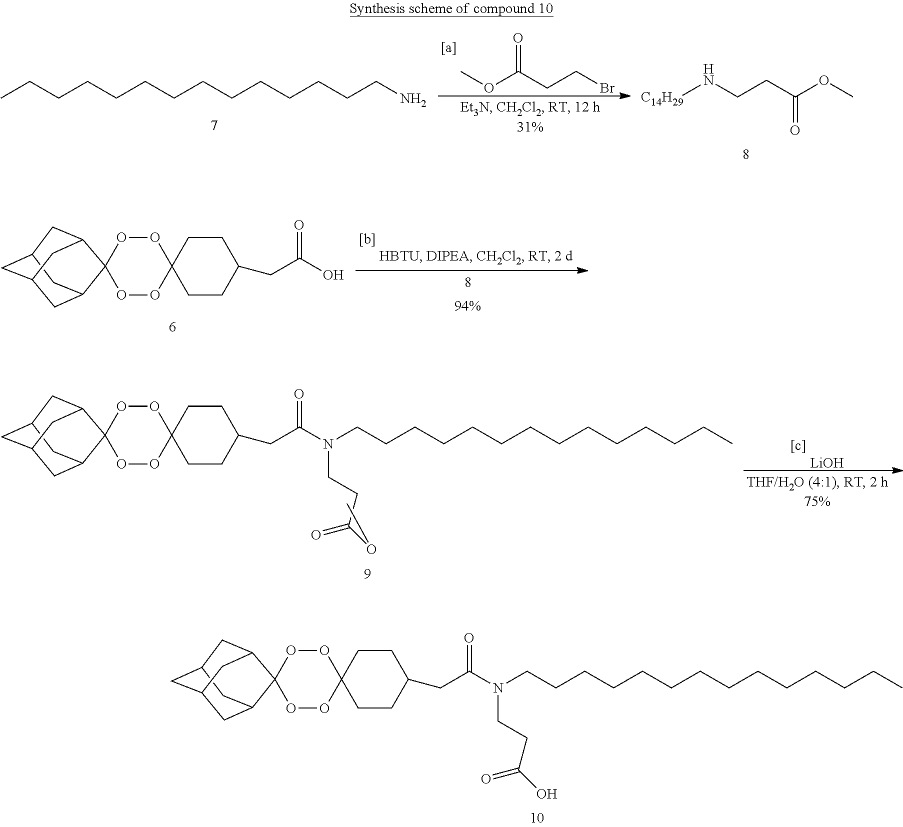

To the solution of tetradecylamine 7 (1.00 g, 4.7 mmol) in anhydrous CH₂Cl₂ (15 mL) was added Et₃N (1.3 mL, 9.37 mmol) and methyl-3-bromopropionate (0.74 mL, 6.6 mmol) sequentially. The reaction mixture was stirred for 12 h. Reaction mixture was filtered through sintered glass to remove Et₃N salt. Solvent was removed under vacuum. The crude product was purified by silica gel column chromatography (n-Hexane/EtOAc=1:1) to afford 8 (0.25 g, 31% yield) as a yellow solid. To the solution of compound 6 (0.016 g, 0.044 mmol) in anhydrous CH₂Cl₂ (1 mL) was added DIPEA (0.2 mL) and HBTU (0.053 g, 0.13 mmol). After 5 min, 8 (0.0 14 g, 0.044 mmol) was added. The cloudy reaction mixture was stirred for 2 d. Water (5 mL) was added. The aqueous layer was extracted with CH₂Cl₂ (20×3 mL). The combined organic layer was washed with brine, dried over anhydrous sodium sulfate and concentrated under reduced pressure. The residue was purified by silica gel column chromatography (n-Hexane/EtOAc=6:1) to afford compound 9 as colourless oil (0.027 g, 94% yield). To the solution of compound 9 (0.027 g, 0.043 mmol) in THF:H₂O (4:1, 0.5 mL) was added LiGH (0.052 g, 0.22 mmol). Reaction mixture was stirred under RT for 2 h. Water (5 mL) was added. The aqueous layer was acidified with 1M HCl, then extracted with EtOAc (20×3 mL). The combined organic layer was washed with brine, dried over anhydrous sodium sulfate and concentrated under reduced pressure. The residue was purified by silica gel column chromatography (n-Hexane/EtOAc=1:1) to afford compound 10 as colourless oil (0.020 g, 75% yield).

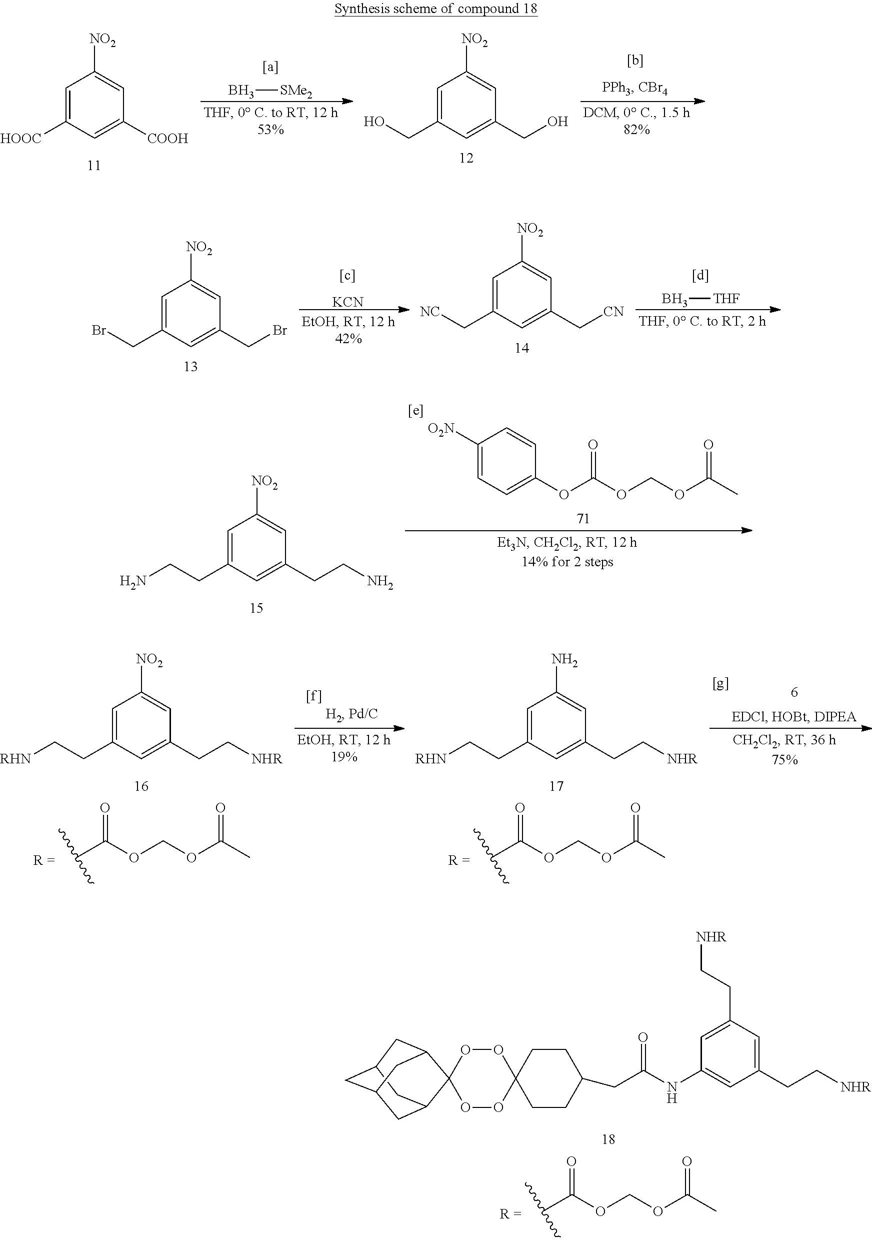

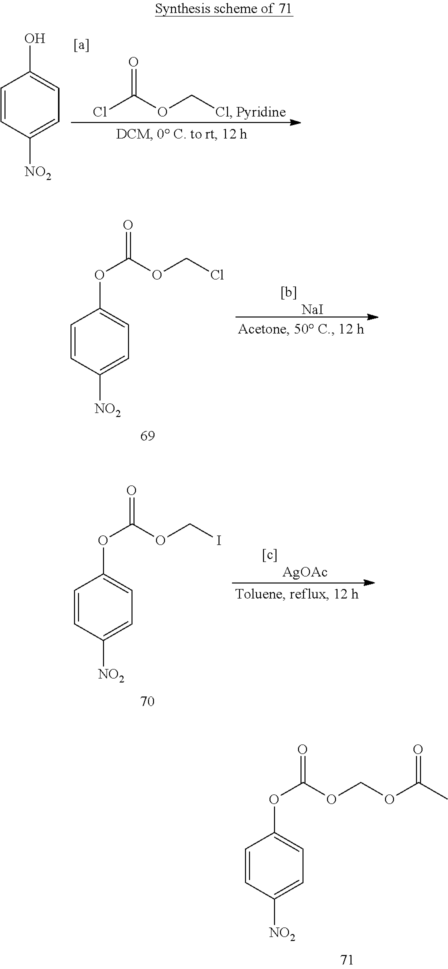

Chloromethyl chloroformate (3.2 mL, 36.3 mmol) was added to an ice-cold solution of p-nitrophenol (5.00 g, 35.9 mmol) in CH₂Cl₂ (60 mL), followed by drop wise addition of pyridine (4.3 mL, 53.9 mmol) over a period of 20 min. The mixture was stirred in the ice-cold bath for 15 min, and then at rt overnight. The reaction mixture was sequentially washed with water (10 mL×2), 1N HCl (10 mL×2), saturated NaHCO₃ solution and brine. After dried over anhydrous sodium sulfate the organic solution was concentrated under reduced pressure and the residue was purified by silica gel column chromatography (EtOAc/n-Hexane=1/3) to give 69 as a white solid (4.5281 g, 54% yield). To solution of 69 (1.500 g, 6.5 mmol) in acetone (40 mL) was added sodium iodide (1.070 g, 7.1 mmol). The mixture was stirred at 50° C. overnight. Solvent was evaporated. The residual material was transferred into diethyl ether and washed with a saturated NaHCO₃ solution. After dried over anhydrous sodium sulfate the organic solution was concentrated to afford the crude product 70, which was immediately dissolved in toluene (9 mL). To this solution was added silver acetate (1.30 g, 7.78 mmol). The mixture was refluxed overnight. The mixture was filtered through a short pad of celite, and the filtrate was evaporated. The mixture was dissolved in diethyl ether, washed with saturated Na₂SO₃ solution and brine. After dried over anhydrous sodium sulfate the organic solution was concentrated under reduced pressure and the residue was purified by silica gel column chromatography (EtOAc/n-Hexane=1/3) to give product 71 as a white solid (0.931 g, 56% yield for two steps). To a solution of 11 (1.50 g, 4.74 mmol) in anhydrous THF (18 mL) at 0° C. was added BH₃—SMe₂ (3.63 mL, 37.7 mmol) dropwisely over 5 min under Ar. The cloudy solution was allowed to warm slowly to room temperature and stirred for 12 h. Methanol (10 mL) was added dropwisely into the reaction mixture until it turned red. Solvent was evaporated under reduced pressure, and the residue was re-dissolved in ethyl acetate, washed with saturated NaHCO₃, water and brine. After dried over anhydrous sodium sulfate the organic solution was concentrated under reduced pressure and the residue was purified by silica gel column chromatography (5% EtOH in CH₂Cl₂) to give 12 as a white solid (0.69 g, 53% yield). To a solution of 12 (0.44 g, 2.4 mmol) in anhydrous CH₂Cl₂ (15 mL) in 0° C. was added triphenylphosphine (1.89 g, 7.19 mmol) and CBr₄ (2.39 g, 7.19 mmol) subsequently under Ar. The yellow solution was allowed to stir at 0° C. for 1.5 h. Water (10 mL) and diethyl ether (100 mL) were added. Any white solid was filtered through a short pad of celite. The aqueous layer was back-extracted with diethyl ether for 3 times. The combined organic layer was washed with brine, dried over anhydrous sodium sulfate and concentrated under reduced pressure. The residue was purified by silica gel column chromatography (EtOAc/n-Hexane=1:4) to give 13 as a white solid (0.61 g, 82% yield). To a stirred solution of 13 (1.00 g, 3.24 mmol) in ethanol (30 mL) at RT was added a solution of potassium cyanide (0.740 g, 11.3 mmol) in water (6.3 mL). The reaction mixture was stirred for 12 h, diluted with water and diethyl ether. Aqueous layer was extracted with ether for 3 times. The organic phase was washed with saturated NaHCO₃ solution, brine, dried over anhydrous sodium sulfate and concentrated under reduced pressure. The residue was purified by silica gel column chromatography (EtOAc/n-hexane=3/1) to give 14 as a white solid (0.274 g, 42% yield). To a stirred solution of 14 (0.100 g, 0.5 mmol) in anhydrous tetrahydrofuran (3 mL) at 0° C. under Ar was added borane-tetrahydrofuran complex solution (1.00 M in tetrahydrofuran; 5 mL, 4.97 mmol). The resulting mixture was warmed to RT and stirred for 2 h. At 0° C., 3N HCl (3 mL) was added dropwisely. EtOAc was added to dilute the mixture. 2N NaOH (15 mL) was added. Aqueous layer was back-extracted with EtOAc (30 mL×3). The combined organic layer was washed by brine, dried over anhydrous Na₂SO₄, filtered and concentrated under reduced pressure to provide of crude product 15, which was carried on to the next step. Product 15 (0.048 g, 0.23 mmol) was dissolved in anhydrous DMF (1.5 mL). To this solution were added 71 (0.122 g, 0.48 mmol) and Et₃N (0.2 mL) sequentially in an argon atmosphere. The reaction mixture was stirred for 12 h. Excess 71 was added until starting material was consumed completely as monitored by TLC (EtOAc/n-hexane=3:2). EtOAc (60 mL) was added. The organic layer was washed with 1 M K₂CO₃ solution (3×10 mL), brine, dried over anhydrous Na₂SO₄, filtered and concentrated to afford the crude product, which was purified by flash column chromatography (EtOAc/n-hexane=3:2) to afford the product 16 as colourless oil (0.022 g, 13% yield for 2 steps). Product 16 (0.022 g, 0.050 mmol) was pre-mixed with palladium on charcoal (0.053 g of a 10% by weight solid). Reaction solvent EtOH (1 mL) was put under vacuum and back-filled with hydrogen gas three times before adding into the reaction mixture. Et₃N (7 μL) was added. The reaction flask was then evacuated and back-filled with hydrogen gas three times before leaving to stir under an atmosphere of hydrogen (balloon). The reaction was monitored by TLC (n-Hexane/EtOAc=1:1). 16 was consumed after 2 h. The reaction mixture was filtered through a pad of Celite, which was then rinsed with EtOH. After the removal of solvent under reduced pressure, the crude product was purified by preparative TLC (80% EtOAc in n-hexane) to afford 17 (0.015 g, 71% yield) as yellow oil. To a solution of compound 6 (0.017 g, 0.050 mmol) in anhydrous CH₂Cl₂ (1 mL) was added DIPEA (19.1 μL, 0.11 mmol). After 5 min, EDCI (0.017 g, 0.11 mmol), HOBt (0.023 g, 0.11 mmol) and 17 (19.1 μL, 0.11 mmol) in anhydrous CH₂Cl₂ (1 mL) were added subsequently. The reaction mixture was stirred at room temperature for 36 h. Excess amounts of EDCI and 6 were added until TLC (EtOAc/n-Hexane=1:1) showed that starting material was consumed completely. The reaction was quenched by NH₄Cl (10 mL) and diluted with CH₂Cl₂. The aqueous layer was back-extracted with CH₂Cl₂ (3×30 mL). The combined organic phase was washed with brine (10 mL), dried over Na₂SO₄, filtered and concentrated under reduced pressure to give crude product, which was purified by preparative TLC (EtOAc/n-Hexane=3:2) to afford compound 18 (0.019 g, 75% yield) as foam.

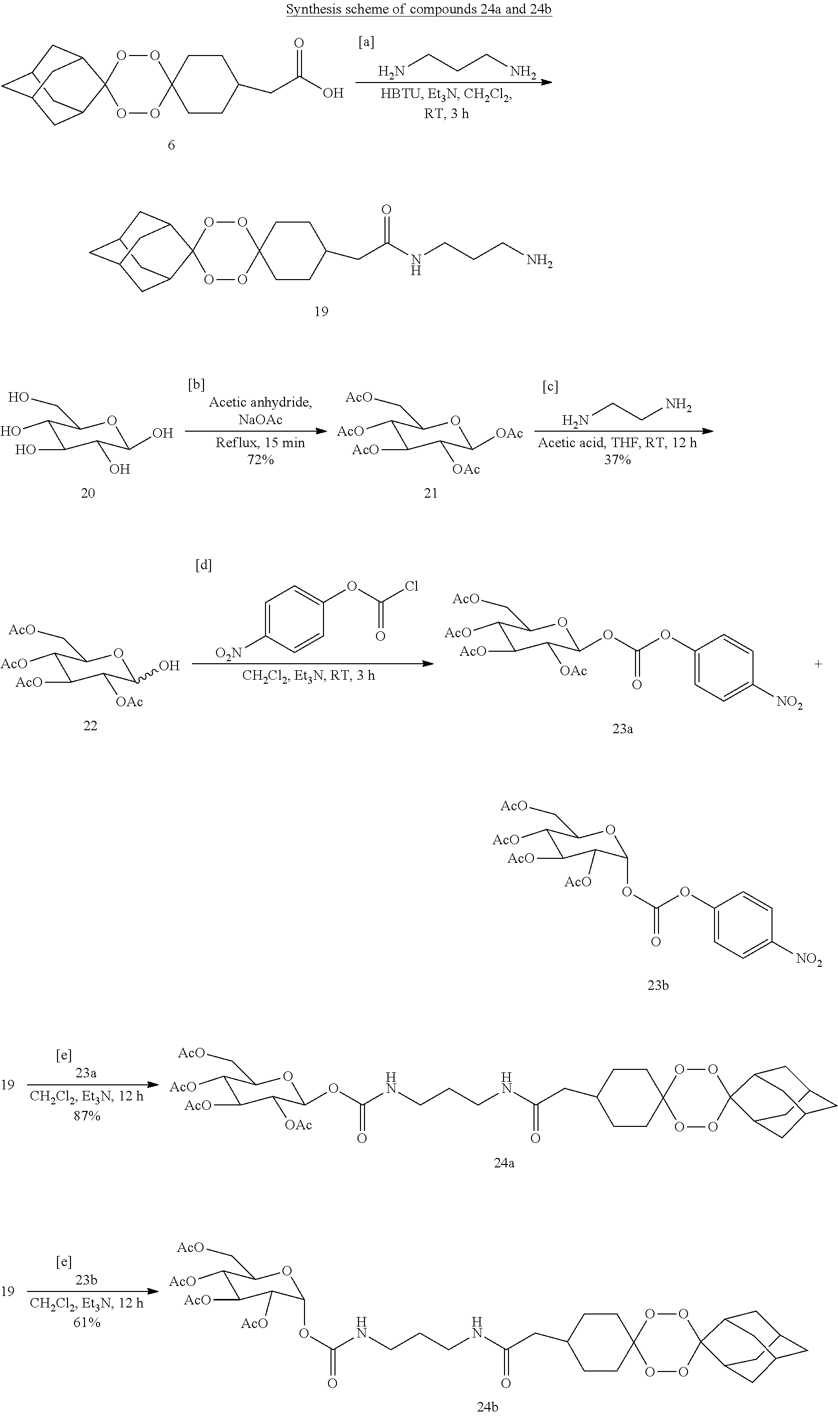

21 was prepared from D-glucose (3.00 g, 16.7 mmol) according to literature report and isolated with recrystallization as white powder (4.70 g, 72% yield) (Huo, et al. Chem Res Toxicol. 2004, 17, 8, 1112-1120). 22 was prepared from 21 (4.669 g, 11.9 mmol) according to literature report and isolated after purification by flash column chromatography (1.560 g, 37% yield) as pale yellow syrup (Huo, et al. Chem Res Toxicol. 2004, 17, 8, 1112-1120). 23 was prepared from 22 (0.305 g, 0.88 mmol) according to literature report with modification and isolated by silica gel column chromatography (EtOAc/n-Hexane=1/4) to give white powder (0.31 g, 69% yield, mixture of α and β isomers) (Cai, et al. J Org Chem. 2005, 70, 9, 3518-3524). α and β isomers were separated by silica gel chromatography with gradient elution (diethyl ether/n-hexane=1/4) to afford α-isomers (0.074 g) and β isomers (0.022 g). To the solution of compound 6 (0.030 g, 0.089 mmol) in anhydrous CH₂Cl₂ (1 mL) were added Et₃N (50 μL, 0.18 mmol) and HBTU (0.037 g, 0.098 mmol). After 5 min, 1,3-diaminopropane (7.4 μL, 0.089 mmol) was added. Reaction mixture was stirred for 12 h. Water (5 mL) was added. The aqueous layer was extracted with CH₂Cl₂ (20×3 mL). The combined organic layer was washed with brine, dried over anhydrous sodium sulfate and concentrated under reduced pressure. The residue 19 was used directly in the next step. To a stirred solution of 23b (0.022 g, 0.043 mmol) in anhydrous DCM (2 mL) was added 9 (0.019 g, 0.047 mmol) and Et₃N (20 μL). Reaction mixture was stirred for 4 h. Solvent was removed by reduced pressure. The residue was purified by silica gel column chromatography to afford compound 24b as colourless oil (0.029 g, 87% yield). To a stirred solution of 23a (0.052 g, 0.10 mmol) in anhydrous DCM (3 mL) was added 9 (0.044 g, 0.11 mmol) and Et₃N (40 μL). Reaction mixture was stirred for 48 h. DMAP was then added. Solvent was removed by reduced pressure. The residue was purified by silica gel column chromatography to afford compound 24a as white foam (0.048 g, 61% yield).

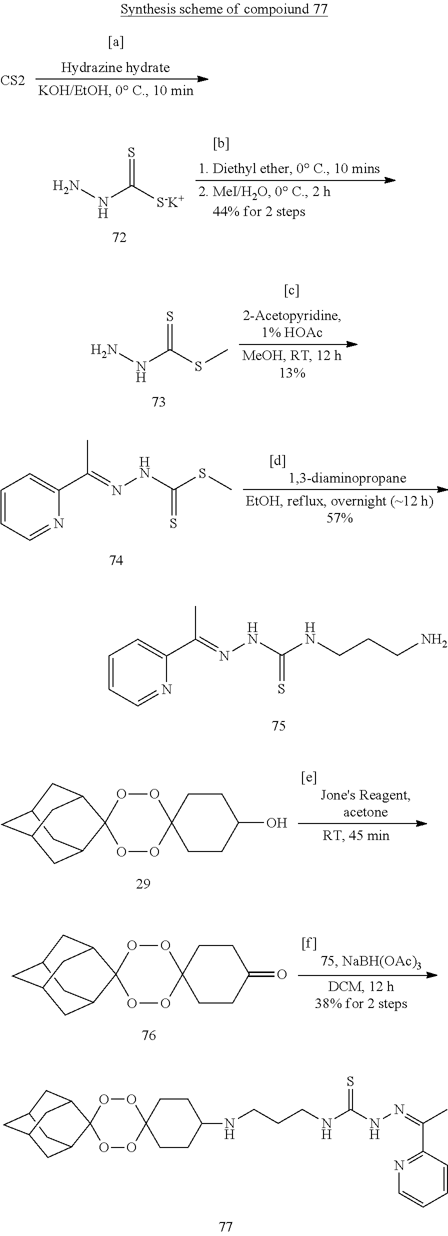

72 was prepared from carbon disulfide (1.58 mL, 26.3 mmol) according to literature report and used immediately for the next step (Pervez, et al. Nat Prod Res. 2007, 1, 13, 1178-1186). 73 was prepared from 72 (26.3 mmol) according to literature report and isolated without purification as a white solid (1.40 g, 44% yield for 2 steps) (Pervez, et al. Nat Prod Res. 2007, 1, 13, 1178-1186). 74 was prepared from 73 (0.30 g, 2.5 mmol) according to literature report and isolated after purification by flash column chromatography (4% EtOH in DCM) as dark green solid (0.074 g, 13% yield) (Greenbaum, et al. J Med Chem. 2004, 47, 12, 3212-3219). To a solution of 74 (0.030 g, 0.13 mmol) in EtOH (2 mL) was added 1,3-diaminopropane (11.1 μL). Reflux was set up and reaction mixture was stirred for overnight. EtOH was removed under reduced pressure. Crude product was purified by silica gel column chromatography (EtOH/DCM, 1% NH₄OH) to afford 75 (0.020 g, 57% yield) as yellow oil. To a solution of 29 (0.030 g, 0.10 mmol) in acetone (0.5 mL) was added Jone's reagent (40 μL). Reaction mixture was stirred for 30 min at RT. EtOAc and H₂O were added. Saturated NaHCO₃ was added until the pH in aqueous layer became 7. EtOAc was used to extract the crude product, washed by brine, then dried by MgSO₄. 76 in EtOAc was filtered through a short pad of silica gel. Solvent was removed under reduced pressure. 76 was used immediately for the next step without purification. Crude 76 was dissolved in DCM (1 mL). This solution was added to 75 (0.016 g, 0.064 mmol) in DCM and stirred for 30 min. Sodium triacetoxyborohydride (0.020 g, 0.094 mmol) was added. Reaction was stirred for 2 h. i-PrOH (0.2 mL) was added. Saturated NH₄Cl was added. DCM was used to extract from the aqueous layer. The combined organic layer was washed by brine, followed by MgSO₄ drying. Concentrated filtrate was purified by silica gel column chromatography (4% EtOH in DCM) to afford compound 77 (0.020 g, 87% yield).

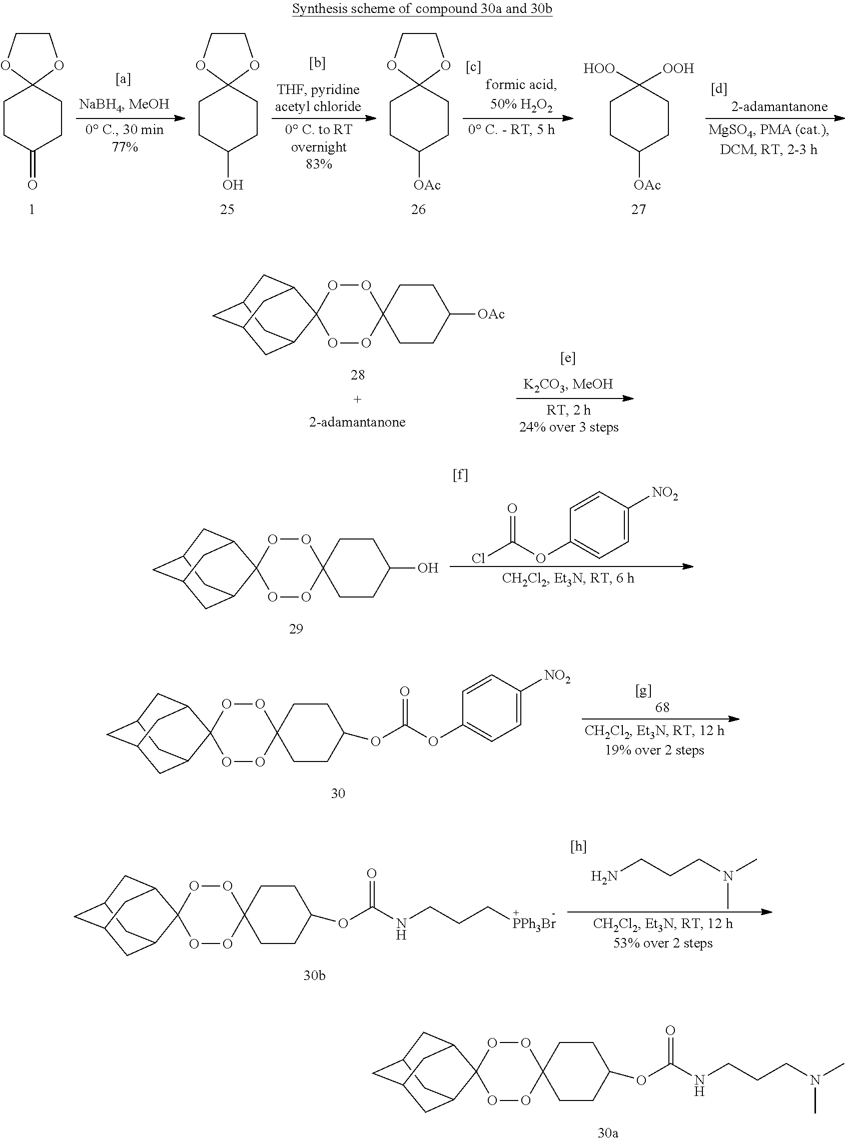

25 was prepared according to literature report²¹ and isolated after purification by flash column chromatography as oil (Uyanik, et al. J Am Chem Soc. 2009, 131, 1, 251-262). 26 was prepared according to literature report and isolated after purification by flash column chromatography as oil (Kovalenko, et al. Chemistry 2015, 21, 7, 2785-2788). Tetraoxane 29 were prepared according to literature report with modifications (O'Neill, et al. Angew Chem Int Ed. 2010, 49, 5693-5697; Yan, et al. Synlett. 2011, 2827-2830). To a stirred solution of 26 (0.70 g, 3.5 mmol) in acetonitrile (3.8 mL) at 0° C. was added formic acid (2.6 mL, 69 mmol), followed by 50% aqueous hydrogen peroxide (1.3 mL, 46 mmol). The mixture was allowed to stir at room temperature. TLC analysis indicated (n-Hexane/EtOAc=2:1) acetate 26 was consumed after 4 h. 1 mL 50% H₂O₂ was added. After 1 h, the mixture was then poured into ice-cold water and extracted with CH₂Cl₂. The combined organic extracts were washed with brine, dried over Na₂SO₄, filtered and concentrated under reduced pressure to give the corresponding gem-dihydroperoxide 27, which was used in the next step immediately. A mixture of 2-adamantanone (0.79 g, 5.2 mmol), PMA (0.0627 g, 1 mol %), and anhydrous MgSO₄ (0.63 g, 5.2 mmol) in CH₂Cl₂ (13 mL) was stirred for 30 min at RT. To this solution was added 27 (3.5 mmol) in CH₂Cl₂ (13 mL) dropwisely in 15 min. The mixture was stirred at RT and monitored by TLC (n-Hexane/EtOAc=4:1). When 27 was consumed completely, deionized H₂O (10 mL) was added. The aqueous layer was extracted with CH₂Cl₂ (3×10 mL). The combined organic phase was washed by brine, dried by Na₂SO₄ and concentrated under reduced pressure. The residue was purified by silica gel column chromatograph (n-hexane/EtOAc=8:1) to afford a mixture of 28 and 2-adamantanone. Isolation of analytically pure 28 by flash chromatography on silica gel was not possible since it co-elutes with 2-adamantanone. Therefore the product mixture was carried forward to the next step. To a solution of a mixture of 28 and 2-adamantanone (0.94 g total) in MeOH (62 mL) was added K₂CO₃ (2.64 g, 19.1 mmol) at RT. Water (20 mL) was added CH₂Cl₂ (3×30 mL) was used to extract the product from the aqueous layer. The combined organic phases were washed with brine (2×10 mL), dried over Na₂SO₄, filtered and concentrated under reduced pressure. The residue was purified by silica gel column chromatography (n-hexane/EtOAc=2/1) to afford 29 (0.252 g, 24% yield for 3 steps) as a white solid. To a solution of 29 (0.014 g, 0.049 mmol) in anhydrous dichloromethane (3 mL) were added p-nitrophenyl chloroformate (0.098 g, 0.49 mmol) and Et₃N (0.1 mL, 0.71 mmol) sequentially in an argon atmosphere. The reaction mixture was stirred 12 h. DCM (60 mL) was added and the organic layer was washed by 1 M K₂CO₃ solution (3×10 mL), dried over anhydrous Na₂SO₄, filtered and concentrated to afford the crude product. Crude product was dissolved in anhydrous dichloromethane (1 mL). To this solution were added 68 (0.021 g, 0.044 mmol) and Et₃N (22 μL, 0.16 mmol) sequentially in an argon atmosphere. The reaction mixture was stirred for 12 h. Dichloromethane (10 mL) was added. The organic layer was washed with 1 M K₂CO₃ solution (3×5 mL), dried over anhydrous Na₂SO₄, filtered and concentrated to afford the crude product, which was purified by flash column chromatography to afford the compound 30b as yellow oil (0.068 g, 19% yield for 2 steps). Following the stoichiometric ratio and procedure for the synthesis of compound 30b, compound 30a was obtained from 29 (0.011 g, 0.037 mmol) and 3-(Dimethylamino)-1-propylamine (5 μL, 0.037 mmol) as colourless oil (0.0083 g, 53% yield for 2 steps) (7% EtOH in DCM, 1% NH₄OH).

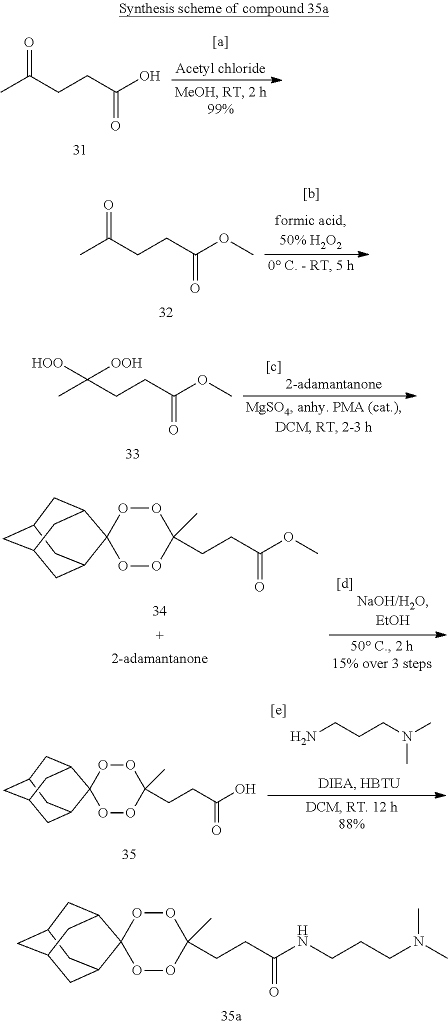

32 was prepared according to literature report and isolated without purification as oil (An, et al. Chemistry 2015, 21, 30, 10786-10798). Following the stoichiometric ratio and procedure for the synthesis of 6, 35 was obtained from 2-adamantanone (1.90 g, 12.7 mmol) and 32 (1.50 g, 11.5 mmol) as white solid (0.52 g, 15% yield for 3 steps) (EtOAc/n-hexane=1/3). Following the stoichiometric ratio and procedure for the synthesis of compound 6a, compound 35a was obtained from 35 (0.009 g, 0.031 mmol) and 3-(Dimethylamino)-1-propylamine (3.4 μL, 0.031 mmol) as yellow oil (0.010 g, 88% yield) (7% EtOH in DCM, 1% NH₄OH).

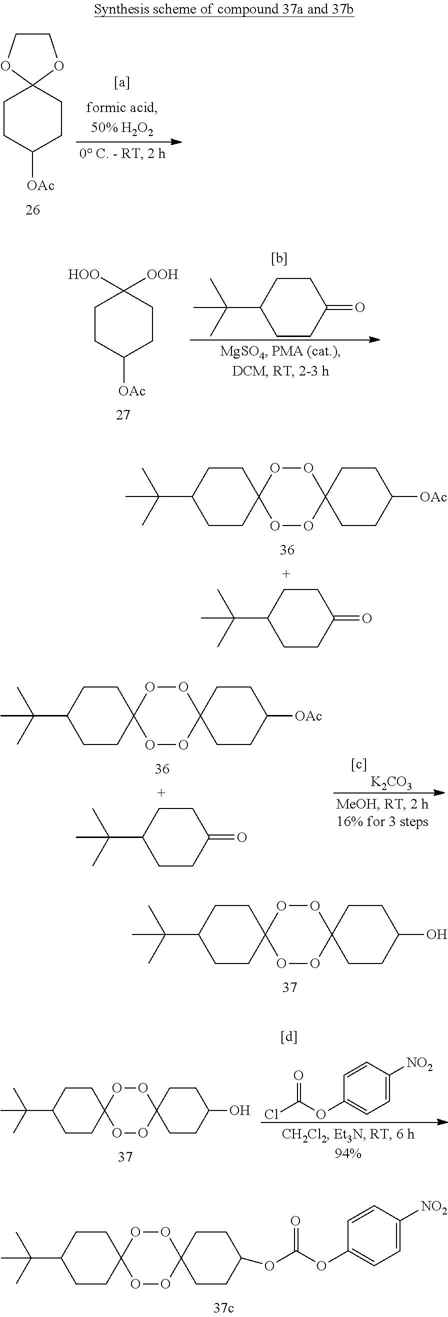

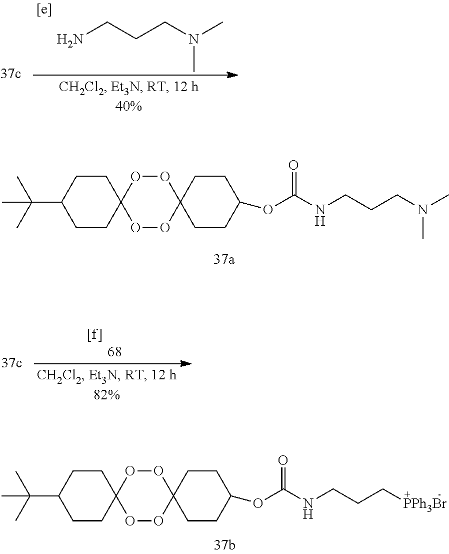

Following the stoichiometric ratio and procedure for the synthesis of 29, 37 was obtained from compound 4-tert-butylcyclohexanone (0.57 g, 3.7 mmol) and 26 (0.73 g, 3.6 mmol) as white solid (0.18 g, 16% yield for 3 steps) (n-hexane/EtOAc=2/1). 37 appeared as a mixture of diastereomers (dr). Following the stoichiometric ratio and procedure for the synthesis of compound 30b, compound 37c was obtained from 37 (0.0367 g, 1.2 mmol) and p-nitrophenyl chloroformate (0.738 g, 3.7 mmol) as white solid (0.534 g, 94% yield) (EtOAc/n-hexane=1/8). Compound 37c appeared as a mixture of diastereomers (dr). Following the stoichiometric ratio and procedure for the synthesis of compound 30a, compound 37a was obtained from compound 37c (0.019 g, 0.04 mmol) and 3-(dimethylamino)-1-propylamine (28.5 μL, 0.044 mmol) as yellow oil (0.007 g, 40% yield for two steps) (3% EtOH in DCM). Compound 37a appeared as a mixture of diastereomers (dr). Following the stoichiometric ratio and procedure for the synthesis of compound 30b, compound 37b was obtained from compound 37c (0.133 g, 0.29 mmol) and 68 (0.137 g, 0.28 mmol) as yellow foam (0.17 g, 82% yield) (3% EtOH in DCM). Compound 37b appeared as a mixture of diastereomers (dr).

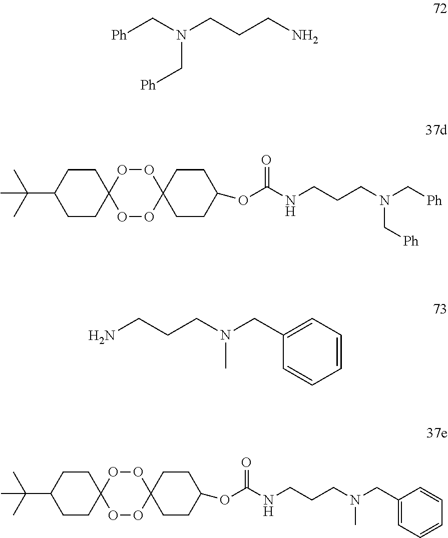 [SMILES: NCCCN(Cc1ccccc1)Cc1ccccc1 ; CC(C)(C)C1CCC2(CC1)OOC1(CCC(OC(=O)NCCCN(Cc3ccccc3)Cc3ccccc3)CC1)OO2 ; CN(CCCN)Cc1ccccc1 ; CN(CCCNC(=O)OC1CCC2(CC1)OOC1(CCC(C(C)(C)C)CC1)OO2)Cc1ccccc1]

72 was prepared according to literature report and isolated after purification by flash column chromatography (5% EtOH in DCM, 1% NH₄OH) as yellow oil (Hou, et al. J Org Chem. 2004, 69, 18, 6094-6099). Following the stoichiometric ratio and procedure for the synthesis of compound 30a, compound 37d was obtained from compound 37c (0.049 g, 0.11 mmol) and 72 (0.053 g, 0.16 mmol) as yellow oil (0.019 g, 30% yield) (1% EtOH in DCM). Compound 37d appeared as a mixture of diastereomers (dr). 73 was prepared according to literature report and isolated after purification by flash column chromatography (5% EtOH in DCM, 1% NH₄OH) as yellow oil (Ghedira, et al. J Med Chem. 2018, 158, 51-67). Following the stoichiometric ratio and procedure for the synthesis of compound 30a, compound 37e was obtained from compound 37c (0.031 g, 0.066 mmol) and 73 (0.012 g, 0.066 mmol) as yellow oil (0.013 g, 40% yield) (1% EtOH in DCM). Compound 37e appeared as a mixture of diastereomers (dr).

### Synthesis Scheme for Compound 37g

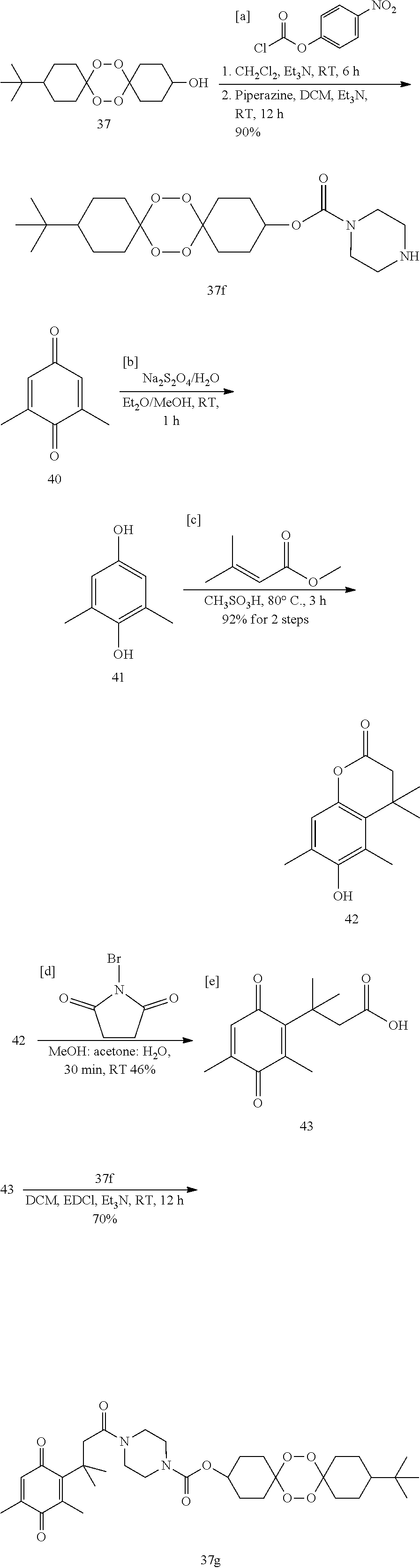

Compound 37c (0.031 g, 0.066 mmol) was dissolved in anhydrous dichloromethane (1 mL). To this solution were added piperazine (0.011 g, 0.133 mmol) and Et₃N (60 μL, 0.4 mmol) sequentially in an argon atmosphere. The reaction mixture was stirred for 12 h. Dichloromethane (60 mL) was added. The organic layer was washed with 1 M K₂CO₃ solution (3×10 mL), dried over anhydrous Na₂SO₄, filtered and concentrated to afford the crude product, which was purified by flash column chromatography to afford the product 37f as yellow oil (0.025 g, 90% yield). Compound 37f appeared as a mixture of diastereomers (dr). 41 was prepared according to literature report and isolated as an off-white solid without any further purification and characterization (Ji, et al. J. ACS Med Chem Lett. 2015, 6, 6, 707-710). 42 was prepared according to literature report and isolated as an off-white solid without any further purification and characterization (Ji, et al. J. ACS Med Chem Lett. 2015, 6, 6, 707-710). 43 was prepared according to literature report and isolated after purification by flash column chromatography as a yellow solid (Ji, et al. J. ACS Med Chem Lett. 2015, 6, 6, 707-710). To a solution of 43 (0.008 g, 0.035 mmol) in anhydrous CH₂Cl₂ (1 mL) were added Et₃N (12 μL, 0.087 mmol) and HBTU (0.020 g, 0.052 mmol) subsequently. After 5 min, compound 37f (0.014 g, 0.035 mmol) was added. Reaction mixture was stirred at room temperature for 12 h. Solvent was removed under reduce pressure when TLC (EtOAc/n-Hexane=1:1) showed that 43 was consumed completely. The crude product was purified by preparative TLC (EtOAc/n-Hexane=1:4) to afford compound 37g (0.016 g, 70% yield) as bright yellow oil. Compound 37g appeared as a mixture of diastereomers (dr).

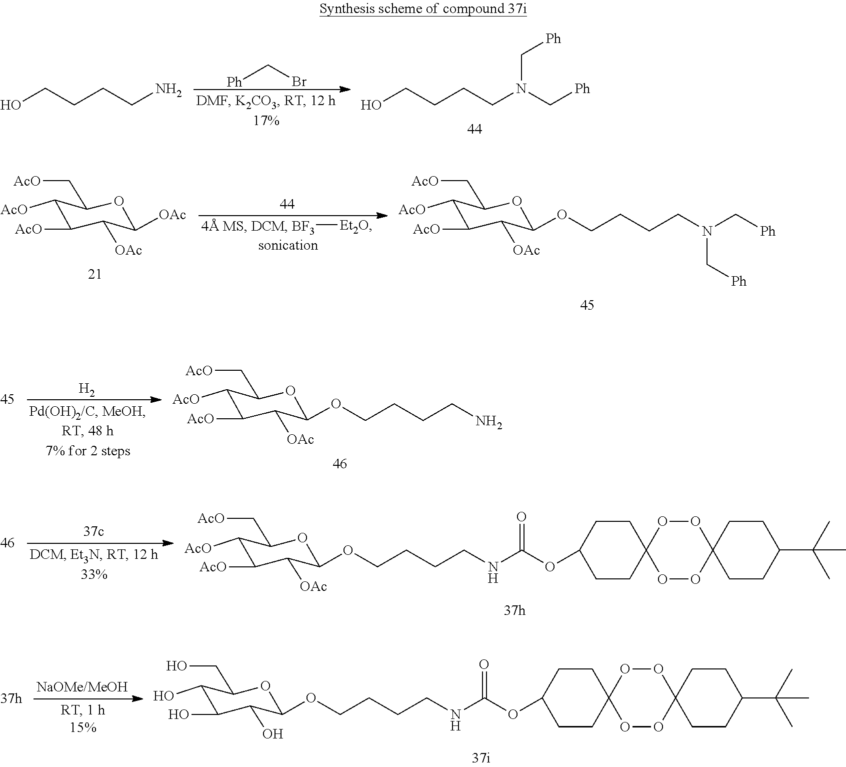

44 was prepared from 4-amino-1-butanol (0.5 mL, 5.4 mmol) according to literature report and isolated after purification by flash column chromatography (EtOAc/n-Hexane=1:4) as bright yellow oil (0.255 g, 17%) (De, et al. J Enzyme Inhib Med Chem. 2016, 31, 106-113). To a solution of 1,2,3,4,6-penta-O-acetyl-D-pyranoglucose (0.349 g, 0.89 mmol) in anhydrous DCM with 4 Å MS were added 44 (0.255 g, 0.95 mmol) and BF₃·Et₂O (0.55 mL, 4.5 mmol) subsequently. Reaction mixture was stirred at room temperature with sonication until the complete consumption of the starting material (monitored by TLC) (Deng, et al. J Org Chem. 2006, 71, 5179-5185). The reaction mixture was quenched by addition of NaHCO₃ then filtered through Celite. The filtrate was concentrated under reduced pressure to afford crude product 45 which was used for the next step without further purification. To a solution of crude 45 (0.070 g, 0.93 mmol) in methanol (15 mL) was added 20% wt Pd/C (0.099 g, 10 mol %) with stirring at rt. The air in the flask was replaced with hydrogen, and the mixture was stirred under hydrogen atmosphere for 2 d. Reaction mixture was filtered through a pad of celite, which was then rinsed with EtOH, and concentrated to afford the crude product, which was purified by flash column chromatography to afford 46 as yellow oil (0.026 g, 7% yield for two steps). Compound 37c (0.0436 g, 0.094 mmol) was dissolved in anhydrous dichloromethane (1 mL). To this solution were added 46 (0.023 mg, 0.055 mmol) and Et₃N (30 μL, 0.22 mmol) sequentially in an argon atmosphere. The reaction mixture was stirred for 12 h. Dichloromethane (60 mL) was added. The organic layer was washed with 1 M K₂CO₃ solution (3×10 mL), dried over anhydrous Na₂SO₄, filtered and concentrated to afford the crude product, which was purified by flash column chromatography to afford compound 37h as yellow oil (0.0133 g, 33% yield). Compound 37h appeared as a mixture of diastereomers (dr). NaOMe in MeOH (0.03M, 0.5 mL) was added into compound 37h (0.013 g, 0.018 mmol) at 0° C. Reaction mixture was stirred under rt for 1 h. Dichloromethane and water were added. The aqueous layer was extracted with DCM (3×10 mL). The combined organic layer was dried over anhydrous Na₂SO₄, filtered and concentrated to afford the crude product, which was purified by flash column chromatography to afford compound 37i as white solid (0.0015 g, 15% yield). Compound 37i appeared as a mixture of diastereomers (dr).

To a solution of 2,2′-(ethylenedioxy)bis(ethylamine) (1.8 μL, 0.0125 mmol) in CH₂Cl₂ was added compound 37c (0.012 g, 0.025 mmol) in CH₂Cl₂, followed by 4-(dimethylamino)pyridine (DMAP) and Et₃N (10 μL, 0.025 mmol) were added. Reaction mixture was stirred at RT for 48 h. Solvent was concentrated under reduced pressure to afford the crude product, which was purified by flash column chromatography to afford compound 37j as oil (0.010 g, 50% yield). Compound 37j appeared as a mixture of diastereomers (dr). To a solution of 37 (0.100 g, 0.32 mmol) in THF (7 mL) at 0° C. was added sodium hexamethyldisilazide [NaHMDS, 1.0 M in THF](0.33 mL, 0.32 mmol). The resulting mixture was stirred for 10 min at 0° C., and the appropriate dichlorophosphate (0.024 mL, 0.16 mmol) was added. The reaction mixture was kept at 0° C. for 2 h and was then warmed to room temperature and stirred for 12 h. The mixture was then cooled back to 0° C., quenched with water. The organic layer was extracted three times with ether, and the combined organic portions were washed with saturated NaCl and dried over MgSO₄. The organic extracts were removed under reduced pressure, and the product was purified by flash column chromatography to afford compound 37k as oil (0.030 g, 26% yield). Compound 37k appeared as a mixture of diastereomers (dr).

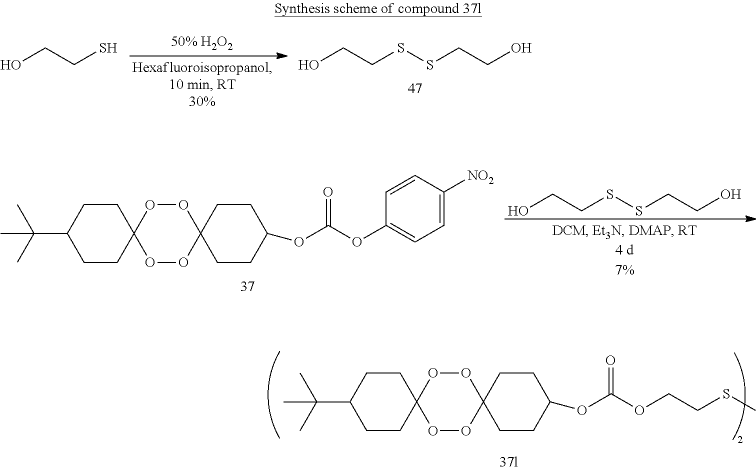

47 was prepared from 2-mercaptoethanol (2 mL, 28.5 mmol) according to literature report and isolated after purification by flash column chromatography (EtOAc/n-hexane=1/1) as oil (1.036 g, 30% yield) (Chen, et al. J. Mater. Sci. 2018, 53 16169-16181). To a solution of compound 37c (0.017 g, 0.036 mmol) in CH₂Cl₂ were added 2,2′-disulfanediylbis (ethan-1-ol) (0.0086 g, 0.018 mmol), Et₃N (10 μL) and DMAP. Reaction was stirred at RT for 48 h. The crude product was purified by preparative TLC by using EtOAc/n-hexane (1:8) to afford compound 37l (0.005 g, 7% yield). Compound 37l appeared as a mixture of diastereomers (dr).

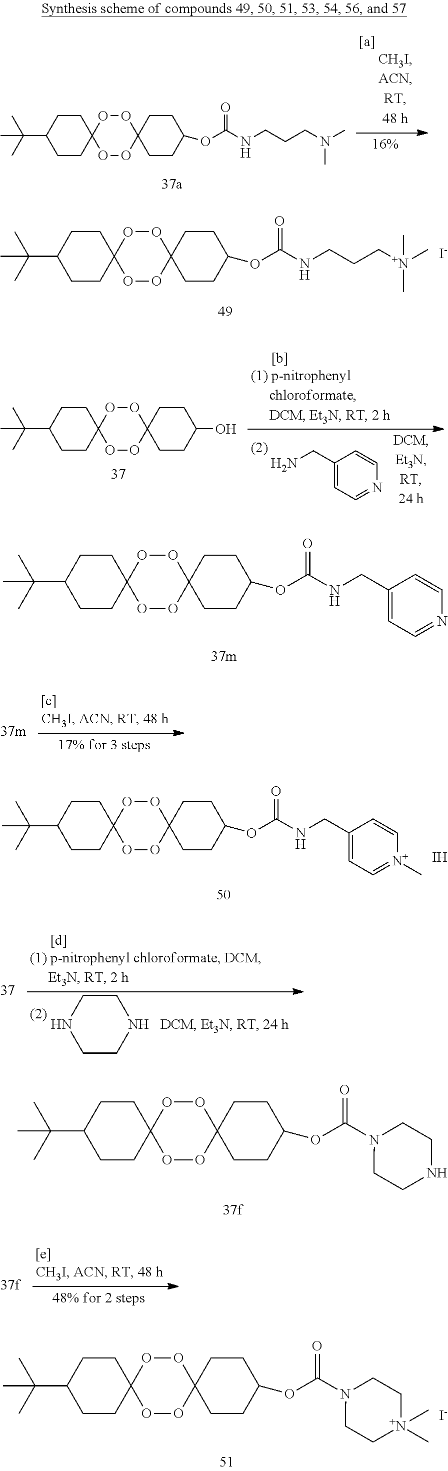

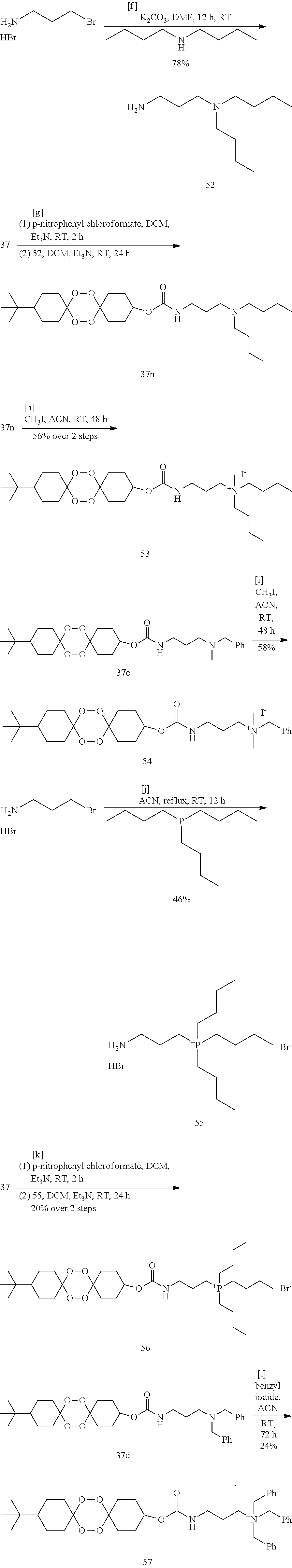

Methyl iodide:acetonitrile (1:1) was added into tertiary amine. Reaction mixture was stirred at RT for 4 to 12 h. UPLC was used to check the conversion. Solvent was removed under reduce pressure. The crude product was purified by flash column chromatography (100% EtOAc, then 4% EtOH/DCM) to afford quaternary ammonium (“QA”) as bright yellow oil. Following the procedure for the synthesis of QA compound, compound 48 was obtained from compound 37d (0.018 g, 0.031 mmol) as oil (0.016 g, 70% yield). Compound 48 appeared as a mixture of diastereomers (dr). 55 was prepared from 3-bromopropylamine hydrobromide (3.00 g, 13.7 mmol) according to literature report and isolated as oil without any further purification and characterization (2.644 g, 46% yield) (Millard, et al. J Med Chem. 2013, 56, 22, 9170-9179). Following the stoichiometric ratio and procedure for the synthesis of compound 37b, compound 56 was obtained from 55 (0.066 g, 0.156 mmol) as oil (0.020 g, 20% yield) (3% EtOH in DCM). Compound 37l appeared as a mixture of diastereomers (dr). Following the stoichiometric ratio and procedure for the synthesis of compound 30a and QA compound, compound 50 was obtained from 37 (0.020 g, 0.067 mmol) as oil (0.006 g, 17% yield for 3 steps) (3% EtOH in DCM). Compound 50 appeared as a mixture of diastereomers (dr). Following the stoichiometric ratio and procedure for the synthesis of compound 30a and QA compound, compound 51 was obtained from 37 (0.030 g, 0.10 mmol) as oil (0.027 g, 48% yield for 2 steps) (3% EtOH in DCM). Compound 51 appeared as a mixture of diastereomers (dr). Following the stoichiometric ratio and procedure for the synthesis of QA compound, compound 49 was obtained from compound 37a (0.042 g, 0.097 mmol) as oil (0.0086 g, 16%) (3% EtOH in DCM). Compound 49 appeared as a mixture of diastereomers (dr). Compound 52 was prepared from 3-bromopropylamine hydrobromide (2.00 g, 9.1 mmol) according to literature report and isolated after purification by flash column chromatography (1.33 g, 78% yield) as oil (5% EtOH in DCM, 1% NH₄OH) (Labadie, et al. Bioorg Med Chem Lett. 2004, 14, 3, 615-619). Following the stoichiometric ratio and procedure for the synthesis of compound 30a and QA compound, compound 53 was obtained from compound 37c (0.031 g, 0.16 mmol) as oil (0.058 g, 55% yield for 2 steps) (3% EtOH in DCM). Compound 53 appeared as a mixture of diastereomers (dr). Following the stoichiometric ratio and procedure for the synthesis of QA compound, compound 54 was obtained from compound 37e (0.0054 g, 0.011 mmol) as oil (0.004 g, 58% yield) (3% EtOH in DCM). Compound 54 appeared as a mixture of diastereomers (dr). Benzyl iodide was prepared according to literature report and isolated as oil without any further purification and characterization (Hoang, et al. J Org Chem. 2009, 74, 11, 4177-4187). To a solution of 37d (0.020 g, 0.034 mmol) in acetonitrile was added benzyl iodide (0.022 g, 0.10 mmol). Reaction mixture was stirred under rt for 72 h. Solvent was removed by reduced pressure. The crude product was purified by silica gel column chromatography (100% EtOAc, then 6% EtOH/DCM) to afford compound 57 (0.0065 g, 24% yield) as bright yellow oil. Compound 57 appeared as a mixture of diastereomers (dr).

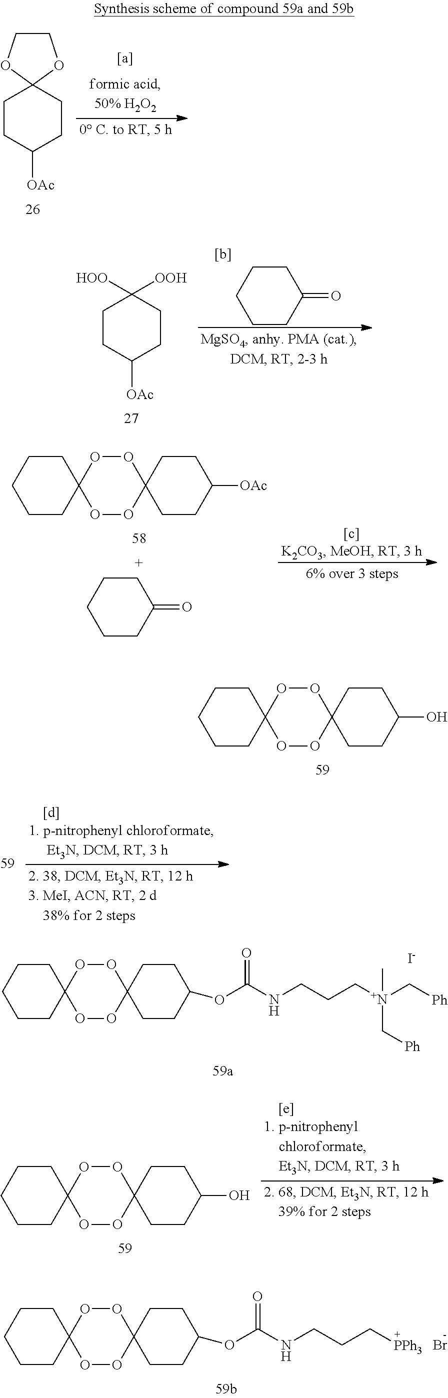

Following the stoichiometric ratio and procedure for the synthesis of compound 30a, 59 was obtained from 26 (2.00 g, 9.9 mmol) and cyclohexanone (0.49 mL, 4.7 mmol) as oil (0.106 g, 9% yield for 2 steps) (EtOAc/n-hexane=1/2). Following the stoichiometric ratio and procedure for the synthesis of compound 30a and QA compound, compound 59a was obtained from 59 (0.028 g, 0.3 5 mmol) and 72 (0.034 g, 0.14 mmol) as yellow oil (0.018 g, 38% yield for 2 steps). Following the procedure for the synthesis of compound 30b, compound 59b was obtained from 59 (0.010 g, 0.023 mmol) and 68 (0.012 g, 0.023 mmol) as white solid (0.006 g, 39% yield for 2 steps) (2% EtOH in DCM).

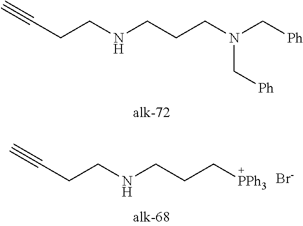

To a solution of 72 (0.113 g, 0.44 mmol) in DCM (1 mL) was added 4-bromobut-1-yne (22.2 μL, 0.24 mmol), followed by Et₃N (47.4 μL, 0.34 mmol). Reaction was stirred at RT for 24 h. Solvent was removed under reduce pressure. The crude product was purified by flash chromatography (20% EtOH in DCM, 1% NH₄OH) to provide alk-72 as oil (0.0154 g, 21% yield). Following the stoichiometric ratio and procedure for the synthesis of 30a and QA compound described above, compound alk-R-48 was obtained from compound 37c (0.0432 g, 0.093 mmol) and alk-72 (0.0154 g, 0.05 mmol) as a yellow oil (0.0071 g, 18% yield for 2 steps) (5% EtOH in DCM). Compound alk-R-48 appeared as a mixture of diastereomers (dr). To a solution of 68 (0.105 g, 0.22 mmol) in DMF (1 mL) was added 4-bromobut-1-yne (20.5 μL, 0.22 mmol), followed by Et₃N (33.8 μL, 0.24 mmol). Reaction was stirred at RT for 24 h. Solvent was removed under reduce pressure. The crude product was purified by flash chromatography (20% EtOH in DCM, 1% NH₄OH) to provide alk-68 as oil (0.0224 g, 23%). Following the stoichiometric ratio and procedure for the synthesis of compound 30a and quaternary amine compound described above, compound alk-R-37b was obtained from compound 37 (0.020 g, 0.067 mmol) and alk-68 (0.022 g, 0.049 mmol) as a yellow oil (0.0067 g, 18% yield for 2 steps) (3% EtOH in DCM). Compound alk-R-37b appeared as a mixture of diastereomers (dr).

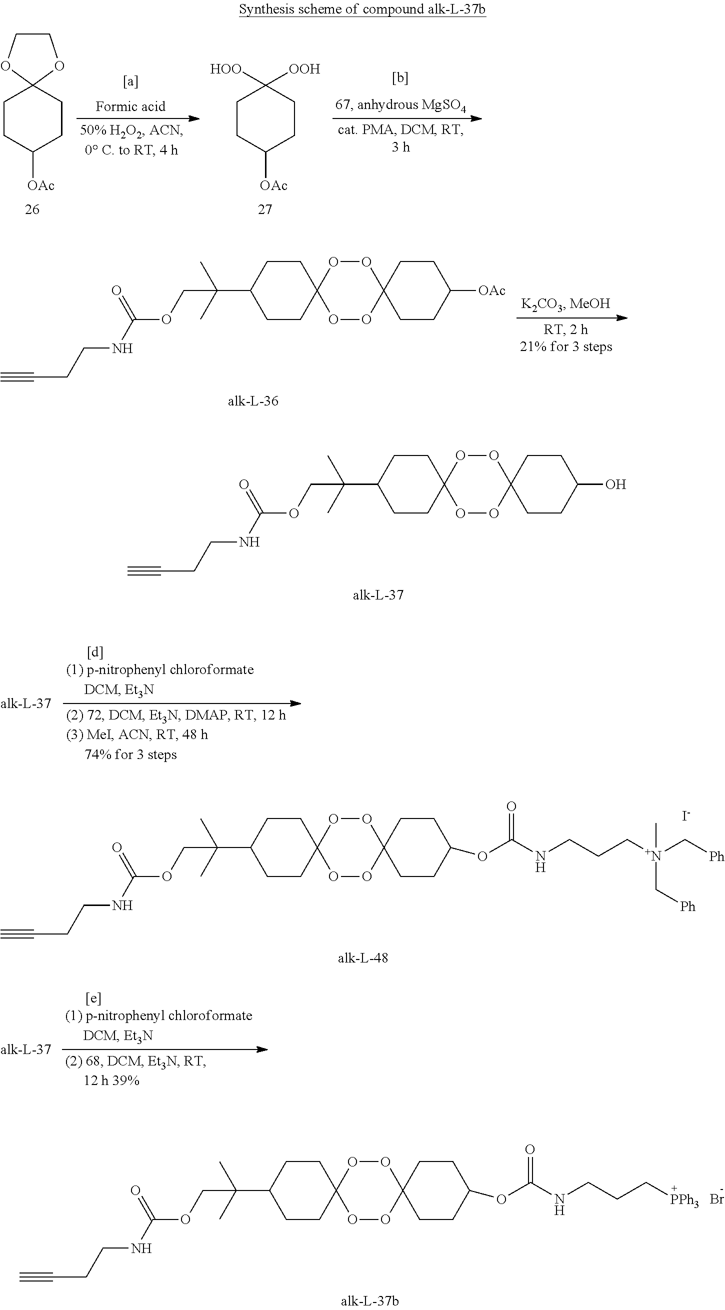

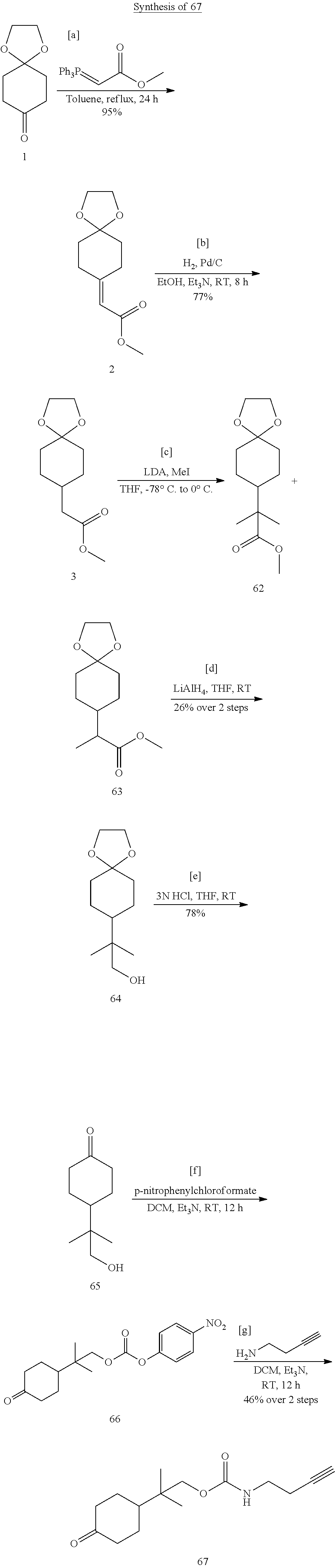

62 and 63 were prepared according to literature report with modifications and isolated after purification by flash column chromatography as mixture products (WO2010039789A1 by Stamford, et al.). Anhydrous N, N-diisopropylamine (DIPA) was freshly prepared by distillation. DIPA (1.3 mL, 9 mmol) in THF (10 mL) was kept at −78° C. under argon. n-Butyllithium in hexane solution (1M, 8.9 mL, 8 mmol) was added dropwisely into the DIPA solution. Reaction mixture was stirred at −78° C. for 30 min. Half of LDA solution was transferred into a round bottom flask purged with argon and kept at 0° C. Methyl ester 3 (0.949 g, 4.4 mmol) was dissolved in THF (8 mL) and kept at 0° C. under argon. This solution was added dropwisely into half LDA solution at −78° C. Reaction mixture was stirred at −78° C. for 15 min. Methyl iodide (0.58 mL, 8.8 mmol) was then added. Reaction mixture was allowed to warm up to 0° C. and stirred for 15 min. Reaction mixture was cooled down to −78° C., then half of the LDA solution was added and stirred for 30 min. Methyl iodide (0.58 mL, 8.8 mmol) was then added. Reaction mixture was allowed to warm up to 0° C. for 5 min, then stirred for 2 h at RT. The mixture was then diluted by diethyl ether and acidified by 1N HCl. Crude product was extracted with diethyl ether (3×30 mL). The combined organic layer was washed with saturated NaHCO₃, followed by brine, dried over Na₂SO₄. The solvent was removed under reduced pressure and the crude product was purified by silica gel column chromatography (n-hexane/EtOAc=4/1) to afford inseparable mixture 62 and 63 (total 0.465 g). 64 was prepared according to literature report with modifications and isolated after purification by flash column chromatography as oil (WO2010039789A1 by Stamford, et al.). The mixture of 62 and 63 (0.387 g) was added dropwisely into a suspension of LiAlH₄ (0.091 g, 2.4 mmol) in THF (20 mL) at 0° C. The reaction was warmed up to rt and stirred for 4 h. Reaction mixture was cooled down to 0° C., then water, NaOH (1N) and water were added sequentially. It was stirred at RT for 15 min. MgSO₄ was added. Solid was filtered off, rinsed by EtOAc. Filtrate was concentrated under reduce pressure and was purified by silica gel column chromatography (n-hexane/EtOAc=4/1) to afford 64 (0.25 g, 26% yield over 2 steps) as colourless oil. 65 (CAS: 156042-33-0) was prepared from 64 (0.415 g, 1.9 mmol) according to literature report³² and isolated after purification by flash column chromatography as oil (0.257 g, 78% yield) (n-hexane/EtOAc=4/1). To a solution of 65 (0.25 g, 1.5 mmol) in anhydrous CH₂Cl₂ (30 mL) were successively added p-nitrophenyl chloroformate (0.89 g, 4.4 mmol) and triethylamine (0.6 mL, 4.4 mmol). After being stirred for 12 h at room temperature, the crude mixture was diluted with another portion of CH₂Cl₂ (50 mL) and then washed with brine and water. The CH₂Cl₂ layer was dried over anhydrous Na₂SO₄ then concentrated. Crude product 66 was used in the next step without further purification. To a stirred solution of crude 66 (1.5 mmol) in anhydrous CH₂Cl₂ (8 mL) was added 1-amino-3-butyne hydrochloride (0.186 g, 1.7 mmol) and Et₃N (0.63 mL, 4.4 mmol). Reaction mixture was stirred for 12 h. The crude mixture was diluted with CH₂Cl₂ (50 mL) and then washed with 1M K₂CO₃, followed by brine and dried over anhydrous Na₂SO₄ then concentrated. Crude product was purified by silica gel column chromatography to afford 67 as colourless oil (0.179 g, 46% yield over 2 steps). Following the procedure for the synthesis of compound 29 described above, compound alk-L-37 was obtained from compound 26 (0.800 g, 3.99 mmol) and 67 (0.179 g, 0.67 mmol) as oil (0.0576 g, 21% yield for 3 steps) (EtOAc/n-hexane=1:3). Compound alk-L-37 appeared as a mixture of diastereomers (dr). Following the procedure for the synthesis of compound 30a and QA compound described above, compound alk-L-48 was obtained from compound alk-L-37 (0.0501 g, 0.12 mmol) and 72 (0.017 g, 0.067 mmol) as an oil (0.0389 g, 74% yield for 3 steps). Compound alk-L-48 appeared as a mixture of diastereomers (dr). Following the procedure for the synthesis of compound 30b described above, compound alk-L-37b was obtained from compound alk-L-37 (0.0501 g, 0.12 mmol) and 68 (0.026 g, 0.054 mmol) as oil (0.0197 g, 39% yield for 2 steps) (3% EtOH/DCM). Compound alk-L-37b appeared as a mixture of diastereomers (dr).

Compound Characterization

Routine NMR spectra were recorded in CDCl3 at ambient temperature on Bruker Avance DPX 300 Fourier Transform Spectrometer, Bruker Avance DRX 400 Fourier Transform Spectrometer or AVIII-600 spectrometers at ambient temperature or temperature stated therein. ¹H NMR spectra were recorded at 400, 500, or 600 MHz, respectively and ¹³C NMR spectra were recorded at 100, 125, or 150 MHz, respectively. Chemical shifts are reported in ppm on the 6 scale, referenced to internal standard tetramethylsilane (δ 0.00) or residual solvent (¹H NMR: δ 7.26 for CDCl₃, 7.16 for C₆D₆, 5.32 for CD₂Cl₂, 2.05 for (CD₃)₂CO or 3.31 for CD₃OD; ¹³C NMR: δ 77.16 for CDCl₃, 128.1 for C₆D₆, 53.84 for CD₂Cl₂, 29.84 and 206.26 for (CD₃)₂CO or 49 for CD₃OD). Two-dimensional NMR spectra were recorded on Bruker Avance DRX 500 Fourier Transform Spectrometer at room temperature or temperature stated therein. The following abbreviations were used in reporting spectra, brs (broad singlet), s (singlet), d (doublet), t (triplet), q (quartet), m (multiplet), dd (double of double). High resolution mass spectra were obtained in Bruker maxis II high Resolution QTOF using ESI mode. All LCMS analysis was carried out on Waters SQ Detector V4.1 SCN 805 equipped with AQUITY UPLC@ BEH C₁₈ column (17 m) with water (containing 0.1% TFA) and acetonitrile at a flow rate of 0.3 mL/min.

Methyl 2-(1,4-dioxaspiro[4.5]decan-8-ylidene)acetate (2): ¹H NMR (400 MHz, CDCl₃) δ 5.58 (s, 1H), 3.89 (s, 4H), 3.59 (s, 3H), 2.93-2.91 (m, 2H), 2.31-2.28 (m, 2H), 1.70-1.68 (m, 4H). ¹³C NMR (100 MHz, CDCl₃) δ 166.84, 160.53, 113.83, 107.89, 64.40, 50.82, 35.72, 34.94, 34.54, 26.01.

Methyl 2-(1,4-dioxaspiro[4.5]decan-8-yl)acetate (3): ¹H NMR (500 MHz, CDCl₃) δ 3.91 (s, 4H), 3.65 (s, 3H), 2.22 (d, J=7.0 Hz, 2H), 1.83-1.80 (m, 1H), 1.72 (d, J=9.8 Hz, 4H), 1.55 (td, J=15, 5.5 Hz, 3H), 1.31 (q, J=10 Hz, 2H). ¹³C NMR (125 MHz, CDCl₃) δ 173.39, 108.74, 64.37, 51.44, 40.87, 34.44, 33.62, 30.16.

2-((1r,3r,5r,7r)-dispiro[adamantane-2,3′-[1,2,4,5]tetraoxane-6′,1″-cyclohexan]-4″-yl)acetic acid (6): ¹H NMR (600 MHz, CDCl₃) δ 3.15 (br, 1H, COOH), 2.29 (brs, 2H), 1.98-1.86 (m, 9H), 1.75-1.61 (m, 12H), 1.31-1.25 (m, 2H). ¹³C NMR (150 MHz, CDCl₃) δ 178.20, 110.63, 107.68, 40.44, 37.08, 34.41, 33.61, 33.28, 31.22, 30.23, 28.97, 28.49, 27.74, 27.18. ESI-HRMS for C₁₈H₂₇O₆[M+H]⁺: calcd. 338.1729, found 338.3400 or C₁₈H₂₆NaO₆ [M+Na]⁺, calcd. 361.1627, found 361.1601.

(3-(bromo-15-azanyl)propyl)triphenylphosphonium bromide (68): ¹H NMR (400 MHz, CD₃OD) δ 8.15-7.55 (m, 15H), 3.71-3.64 (m, 2H), 3.23 (t, J=8.7, 2H), 2.07-2.04 (m, 2H). ¹³C NMR (100 MHz, CD₃OD) δ: 134.93 (d, ⁴J_{C, P}=2.3 Hz), 133.36 (d, J_{C, P}=10.5 Hz), 130.18 (d, ²J_{C, P}=12.6 Hz), 117.65 (d, J_{C, P}=86.6 Hz), 39.16 (d, 2J_{C, P}=21.0 Hz), 20.34, 19.12 (d, J_{C, P}=53.9 Hz). ³¹P NMR (160 MHz, CD₃OD) δ 23.96. ESI-HRMS for C₂₁H₂₄NP ([M−2Br]⁺): calcd. 320.1563, found: 320.1559.

(3-(2-((1r,3r,5r,7r)-dispiro[adamantane-2,3′-[1,2,4,5]tetraoxane-6′,1″-cyclohexan]-4″-yl)acetamido)propyl)triphenylphosphonium bromide (6b): ¹H (600 MHz, CDCl₃) δ 7.83-7.81 (m, 3H), 7.71-7.68 (m, 6H), 7.65-7.61 (m, 6H), 6.75-6.72 (m, 1H), 3.61-3.41 (m, 4H), 3.2-3.05 (m, 4H), 2.16 (d, J=12 Hz, 2H), 1.95-1.85 (m, 10H), 1.69-1.60 (m, 11H). ¹³C NMR (150 MHz, CDCl₃) δ 173.60, 135.52 (d, ⁴J_{C, P}=3 Hz), 133.40 (d, ³J_{C, P}=9 Hz), 130.82 (d, ²J_{C, P}=12 Hz), 117.97 (d, ¹J_{C,P}=86 Hz), 110.42, 107.88, 42.64, 39.18 (d, ²J_{C,P}=17 Hz), 37.11, 34.41, 33.27, 31.36, 30.21, 29.84, 29.09, 27.81, 27.20 (d, J=3 Hz), 22.60 (d, J=3 Hz), 20.26 (d, ¹J_{C, P}=54 Hz). ³¹P NMR (160 MHz, CDCl₃) δ 23.98.

ESI-HRMS for C₃₉H₄₇NO₅P [M]⁺: calcd. 640.3186, found 640.3149.

N-(3-(dimethylamino)propyl)-2-((1r,3r,5r,7r)-dispiro[adamantane-2,3′-[1,2,4,5]t etraoxane-6′,1″-cyclohexan]-4″-yl)acetamide (6a): ¹H (600 MHz, CD₃OD, 320 K) δ 3.20 (t, J=6 Hz, 2H), 3.15-3.07 (m, 2H), 2.4-2.38 (m, 2H), 2.28 (s, 6H), 2.10 (d, J=6 Hz, 2H), 2.02-1.89 (m, 6H), 1.86-1.51 (m, 17H). ¹³C (150 MHz, CD₃OD, 320 K) δ174.98, 111.24, 108.7, 58.09, 45.31, 43.58, 38.44, 37.98, 35.6, 34.12, 34.09, 32.06, 31.46, 29.88, 29.39, 28.76, 28.56, 28.1. ESI-HRMS for C₂₃H₃₉N₂O₅ [M+H]⁺: calcd. 423.2781, found 423.2830.

Methyl 3-(tetradecylamino)propanoate (8): ¹H NMR (400 MHz, CDCl₃) δ 3.61 (s, 3H), 2.81 (t, J=6.5 Hz, 2H), 2.53 (t, J=7.2 Hz, 2H), 2.45 (t, J=6.5 Hz, 2H), 1.68 (s, 1H), 1.42-1.38 (m, 2H), 1.19 (s, 23H), 0.81 (t, J=6.7 Hz, 3H). ¹³C NMR (100 MHz, CDCl₃) δ 173.25, 77.48, 77.36, 77.16, 76.84, 51.52, 49.86, 45.07, 34.52, 31.94, 30.07, 29.70, 29.65, 29.59, 29.38, 27.35, 22.70, 14.10. ESI-HRMS for C₁₈H₃₈NO₂ [M+H]⁺: calcd. 300.2824, found 300.2895.

### Methyl

3-(2-((1r,3r,5r,7r)-dispiro[adamantane-2,3′-[1,2,4,5]tetraoxane-6′,1″-cyclohexan]-4″-yl)-N-tetradecylacetamido)propanoate (9): ¹H (500 MHz, C₆D₆) (343 K) δ 3.52 (br, 2H), 3.38 (s, 3H), 3.05 (br, 3H), 2.51 (br, 6H), 2.15-2.04 (m, 9H), 1.75-1.69 (m, 6H), 1.61-1.51 (m, 11H), 1.38-1.31 (m, 16H), 1.16 (br, 2H), 0.90 (t, J=6.9 Hz, 3H). ¹³C (125 MHz, C₆D₆) (343 K) δ 170.98, 110.44, 108.19, 51.07, 43.3, 39.53, 37.48, 34.28, 33.67, 33.61, 32.32, 30.75, 30.12, 30.08, 30.02, 29.98, 29.74, 28.84, 27.86, 27.83, 27.29, 23.02, 14.13. ESI-HRMS for C₃₆H₆₂NO₇ [M+H]⁺: calcd. 620.4448, found 620.4491.

3-(2-((1r,3r,5r,7r)-dispiro[adamantane-2,3′-[1,2,4,5]tetraoxane-6′,1″-cyclohexan]-4″-yl)-N-tetradecylacetamido)propanoic acid (10): ¹H NMR (500 MHz, CDCl₃) δ 3.58 (t, J=6.7 Hz, 2H), 3.30-3.09 (m, 4H), 2.65-2.57 (m, 2H), 2.22 (brs, 2H), 1.97-1.51 (m, 23H), 1.26 (brs, 22H), 0.88 (t, J=6.9 Hz, 3H). ¹³C NMR (125 MHz, CDCl₃) δ 175.63, 172.78, 110.58, 107.89, 49.40, 46.03, 43.40, 42.87, 39.19, 37.09, 34.40, 34.18, 34.06, 33.49, 33.29-33.27, 32.06, 31.39, 30.23, 29.79, 29.76, 29.68, 29.50, 29.47, 29.41, 28.71, 27.21, 27.19, 26.94, 22.83, 14.26. ESI-HRMS for C₃₅H₆₀NO₇ [M+H]⁺: calcd. 606.4292, found 606.4335.

Chloromethyl(4-nitrophenyl) carbonate (69): ¹H NMR (300 MHz, CDCl₃) δ 8.30 (d, J=8.5 Hz, 2H), 7.42 (d, J=9.2 Hz, 2H), 5.84 (s, 2H). ¹³C NMR (100 MHz, CDCl₃) δ 154.9, 151.1, 145.8, 125.5, 121.7, 72.8. LRMS (EI, 20 eV) m/z (%) 230.9 (M⁺; 6), 152.0 (100) 186.9 (42). HRMS (EI): calcd for C₈H₆O₅NCl (M⁺): 230.9935, found: 230.9937.

(((4-nitrophenoxy)carbonyl)oxy)methyl acetate (71): ¹H NMR (300 MHz, CDCl₃) δ 8.25 (d, J=9.2 Hz, 2H), 7.36 (d, J=9.3 Hz, 2H), 5.84 (s, 2H), 2.14 (s, 3H). ¹³C NMR (100 MHz, CDCl₃) δ 169.2, 155.0, 151.4, 145.6, 125.3, 121.7, 82.4, 20.6. LRMS (EI, 20 eV) m/z (%) 182.0 ([M-CH₂OAc]⁺, 2), 73.0 (100). HRMS (EI): calcd for C₁₀H₉NO₇ ([M-CH₂OAc]⁺): 182.0089, found: 182.0124.

(5-nitro-1,3-phenylene)dimethanol (12): ¹H NMR (400 MHz, CD₃OD) δ 8.13 (s, 2H), 7.71 (s, 1H), 4.71 (s, 4H). ¹³C NMR (100 MHz, CDCl₃) δ 147.0, 143.2, 129.2, 118.3, 61.6. LRMS (EI, 20 eV) m/z (%) 183.0 (M⁺; 59), 137.0 (100), 166.0 (49). HRMS (EI): calcd for C₈H₉NO₄ (M⁺): 183.0532, found: 183.0523.

1,3-bis(bromomethyl)-5-nitrobenzene (13): ¹H NMR (400 MHz, CD₃OD) δ 8.18 (s, 2H), 7.75 (s, 1H), 4.52 (s, 4H). ¹³C NMR (100 MHz, CDCl₃) δ 148.6, 140.4, 135.4, 123.8, 30.8. LRMS (EI, 20 eV) m/z (%) 309.1 (M⁺; 6), 230.1 (100). HRMS (EI): calcd for C₈H₇Br₂NO₂ (M⁺): 308.8844, found: 308.8813.

2,2′-(5-nitro-1,3-phenylene)diacetonitrile (14): ¹H NMR (400 MHz, acetone-d₆) δ 8.27 (s, 2H), 7.92 (s, 1H), 4.23 (s, 4H). ¹³C NMR (100 MHz, acetone-d₆) δ 205.7, 134.6, 134.1, 122.5, 117.4, 23.3. LRMS (EI, 20 eV) m/z (%) 201.2 (M⁺; 50), 175.2 (47), 155.2 (97); HRMS (EI): calcd for C₁₀H₇N₃O₂ (M⁺): 201.1815, found: 201.0530.

(((((5-nitro-1,3-phenylene)bis(ethane-2,1-diyl))bis(azanediyl))bis(carbonyl))bis(oxy))bis(methylene)diacetate (16): ¹H NMR (400 MHz, CDCl₃) δ 7.91 (s, 2H), 7.41 (s, 1H), 5.19 (br, 2H), 3.51 (q, J=6.6 Hz, 4H), 2.92 (t, J=6.7 Hz, 4H), 2.08 (s, 6H). ¹³C NMR (100 MHz, CDCl₃) δ 170.19, 154.79, 148.56, 140.92, 135.73, 122.29, 79.95, 77.48, 77.16, 76.84, 41.81, 35.71, 20.88. ESI-HRMS for C₁₈H₂₄N₃O₁₀ [M+H]⁺: calcd. 442.1383, found 442.1428 or C₁₈H₂₃N₃NaO₁₀ [M+Na]⁺: calcd. 464.1281, found 464.1246.

(((((5-amino-1,3-phenylene)bis(ethane-2,1-diyl))bis(azanediyl))bis(carbonyl))bis (oxy))bis(methylene)diacetate (17): ¹H NMR (400 MHz, CDCl₃) δ 3.43 (q, J=8 Hz, 4H), 2.70 (t, J=6.8 Hz, 4H), 2.10 (s, 6H). ¹³C NMR (100 MHz, CDCl₃) δ 170.28, 154.67, 147.12, 140.16, 119.46, 113.89, 79.97, 42.10, 35.84, 20.99. ESI-HRMS for C₁₈H₂₆N₃O₈[M+H]⁺: calcd. 412.1642, found 412.1688.

(((((5-(2-((1r,3r)-dispiro[adamantane-2,3′-[1,2,4,5]tetraoxane-6′,1″-cyclohexan]-4″-yl)acetamido)-1,3-phenylene)bis(ethane-2,1-diyl))bis(azanediyl))bis(carbonyl))bis(oxy))bis(methylene)diacetate (18): ¹H NMR (600 MHz, CD₂Cl₂) δ 7.4 (s, 1H), 7.25 (s, 2H), 6.79 (s, 1H), 5.64 (s, 4H), 5.11-5.10 (m, 2H), 3.42 (q, J=8 Hz, 4H), 3.13 (br, 2H), 2.76 (t, J=4 Hz, 4H), 2.25 (brs, 2H), 2.07 (s, 6H), 1.96-1.61 (m, 21H). ¹³C NMR (150 MHz, CD₂Cl₂) δ 170.56, 170.34, 154.87, 140.27, 138.95, 125.30, 118.66, 110.69, 107.99, 80.19, 54.20, 54.02, 53.84, 53.66, 53.48, 44.41, 42.37, 37.24, 36.08, 34.72, 34.49, 33.49, 33.45, 31.49, 30.52, 29.24, 28.82, 28.12, 27.58, 21.04. ESI-HRMS for C₃₆H₅₀N₃O₁₃ [M+H]⁺: calcd. 732.3265, found 732.3300 or C₃₆H₄₉N₃NaO₁₃ [M+Na]⁺: calcd. 754.3163, found 754.3119.

(2R,3R,4S,5R,6S)-2-(acetoxymethyl)-6-(((4-nitrophenoxy)carbonyl)oxy)tetrahy dro-2H-pyran-3,4,5-triyl triacetate (23): R isomer: ¹H NMR (400 MHz, CDCl₃) δ 8.29 (d, J=9.2 Hz, 2H), 7.42 (d, J=9.2 Hz, 2H), 5.67 (d, J=7.7 Hz, 1H), 5.31-5.17 (m, 3H), 4.32 (dd, J=12.6, 4.4 Hz, 1H), 4.17 (dd, J=12.6, 2.1 Hz, 1H), 3.90 (ddd, J=9.8, 4.2, 2.2 Hz, 1H), 2.10 (s, 6H), 2.05 (s, 3H), 2.03 (s, 3H). R isomer: ¹H NMR (400 MHz, CDCl₃) δ 8.29 (d, J=9.2 Hz, 2H), 7.42 (d, J=9.2 Hz, 2H), 5.67 (d, J=7.7 Hz, 1H), 5.23 (ddd, J=18.9, 16.0, 9.1 Hz, 3H), 4.32 (dd, J=12.6, 4.4 Hz, 1H), 4.17 (dd, J=12.6, 2.1 Hz, 1H), 3.90 (ddd, J=9.8, 4.2, 2.2 Hz, 1H), 2.10 (s, 6H), 2.05 (m, 6H). ¹³C NMR (100 MHz, CDCl₃) δ 170.76, 169.49, 169.31, 155.00, 150.97, 145.85, 125.55, 121.77, 96.00, 73.08, 72.62, 70.13, 67.53, 61.38, 20.77, 20.69. α isomer: ¹H NMR (400 MHz, CDCl₃) δ 8.30 (d, J=9.2 Hz, 2H), 7.42 (d, J=9.2 Hz, 2H), 6.28 (d, J=3.6 Hz, 1H), 5.56 (t, J=9.9 Hz, 1H), 5.20 (t, J=12H), 5.16 (dd, J=14, 4 Hz, 2H), 4.30 (dd, J=12.4, 3.9 Hz, 1H), 4.32-4.28 (m, 1H), 4.15 (dd, J=12.4, 2.1 Hz, 1H), 2.10 (s, 3H), 2.04-2.05 (m, 6H). ¹³C NMR (100 MHz, CDCl₃) δ 170.69, 170.22, 169.73, 169.49, 155.10, 150.85, 145.78, 125.57, 121.65, 94.22, 70.58, 69.49, 69.23, 67.59, 61.29, 20.80, 20.73, 20.65, 20.59.

(2R,3R,4S,5R,6S)-2-(acetoxymethyl)-6-(((3-(2-((1r,3r,5r,7r)-dispiro[adamantane-2,3′-[1,2,4,5]tetraoxane-6′,1″-cyclohexan]-4″-yl)acetamido)propyl)carbamoyl)oxy)tetra hydro-2H-pyran-3,4,5-triyl triacetate (24b): ¹H NMR (400 MHz, CDCl₃) δ 6.31 (br, 1H), 5.63 (d, J=10 Hz, 1H), 5.53-5.45 (m, 1H), 5.29-5.23 (m, 1H), 5.14-5.07 (m, 2H), 4.31-4.28 (d, J=15 Hz, 1H), 4.12-4.10 (d, J=10 Hz, 1H), 3.84-3.83 (m, 1H), 3.28 (m, 6H), 2.12-1.85 (m, 26H), 1.69-1.60 (m, 11H). ¹³C NMR (100 MHz, CDCl₃) δ 172.96, 170.81, 170.24, 169.56, 154.58, 110.57, 107.77, 92.99, 72.94, 72.53, 70.36, 67.89, 61.55, 43.36, 37.97, 37.04, 36.23, 34.38, 34.14, 33.25, 33.23, 31.23, 30.19, 29.93, 29.80, 28.94, 28.53, 27.88, 27.16, 27.14, 20.87, 20.80, 20.70. ESI-HRMS for C₃₆H₅₃N₂O₁₆ [M+H]⁺: calcd. 769.3317, found 769.3360.

(2R,3R,4S,5R,6R)-2-(acetoxymethyl)-6-(((3-(2-((1r,3r,5r,7r)-dispiro[adamantan e-2,3′-[1,2,4,5]tetraoxane-6′,1″-cyclohexan]-4″-yl)acetamido)propyl)carbamoyl)oxy)tetrahydro-2H-pyran-3,4,5-triyl triacetate (24a): ¹H NMR (400 MHz, CDCl₃) δ 6.21 (s, 2H), 5.7 (br, 1H), 5.44 (t, J=12 Hz, 1H), 5.20-5.05 (m, 2H), 4.25-4.23 (m, 1H), 4.11-4.06 (m, 3H), 3.30-3.12 (m, 7H), 2.11-1.84 (m, 26H), 1.84-1.59 (m, 9H). ¹³C NMR (100 MHz, CDCl₃) δ 173.03, 170.79, 170.30, 169.80, 169.54, 154.51, 110.54, 107.73, 89.99, 70.04, 69.44, 69.39, 68.03, 61.62, 60.51, 43.31, 37.67, 37.01, 35.94, 34.34, 34.14, 33.21, 31.21, 30.18, 30.06, 28.93, 28.50, 27.84, 27.12, 20.81, 20.77, 20.68, 20.65. ESI-HRMS for C₃₆H₅₃N₂O₁₆ [M+H]⁺: calcd. 769.3317, found 769.3358.

1,4-dioxaspiro[4.5]decan-8-ol (25): ¹H NMR (400 MHz, CDCl₃) δ 3.80 (s, 4H), 3.61 (br, 1H), 3.04-3.01 (m, 1H), 1.72-1.66 (m, 4H), 1.54-1.40 (m, 4H). ¹³C NMR (100 MHz, CDCl₃) δ 108.24, 76.84, 67.65, 64.04, 31.72, 31.47.

1,4-dioxaspiro[4.5]decan-8-yl acetate (26): ¹H NMR (400 MHz, CDCl₃) δ 4.74 (br, 1H), 3.84 (s, 4H), 1.93 (s, 3H), 1.64-1.53 (m, 6H), 1.41-1.37 (m, 2H). ¹³C NMR (100 MHz, CDCl₃) δ 170.45, 107.84, 70.13, 64.24, 64.22, 31.25, 28.26, 21.22.

(1r,3r,5r,7r)-dispiro[adamantane-2,3′-[1,2,4,5]tetraoxane-6′,1″-cyclohexan]-4″-ol (29): ¹H NMR (400 MHz, CDCl₃, 298 K) δ3.83 (br, 1H), 3.13 (br s, 1H), 2.63 (br, 1H), 2.11-1.6 (m, 20H). ¹H NMR (500 MHz, CDCl₃, 253 K) δ3.88 (s, 1H), 3.14 (s, 1H), 2.66 (s, 1H), 2.12 (s, 1H), 1.94 (t, J=11.3 Hz, 2H), 1.89-1.86 (m, 6H), 1.82-1.81 (m, 2H), 1.74-1.72 (m, 2H), 1.69-1.64 (m, 3H), 1.62-1.52 (m, 4H). ¹³C NMR (100 MHz, CDCl₃, 298 K) δ 110.57, 107.42, 67.80, 37.01, 34.34, 33.21, 30.20, 29.61, 28.32, 27.12, 25.73. ¹³C NMR (125 MHz, CDCl₃, 253 K) δ 110.65, 107.39, 67.76, 36.66, 34.03, 32.98, 32.96, 32.95, 30.20, 29.83, 29.36, 28.18, 26.75, 25.58. ESI-HRMS for C₁₆H₂₄NaO₅ [M+Na]⁺: calcd. 319.1521, found 319.1498.

(3-(((((1r,3r,5r,7r)-dispiro[adamantane-2,3′-[1,2,4,5]tetraoxane-6′,1″-cyclohexan]-4″-yl)oxy)carbonyl)amino)propyl)triphenylphosphonium bromide (30b): ¹H NMR (400 MHz, CDCl₃) δ 7.8-7.76 (m, 9H), 7.7-7.68 (m, 6H), 6.75 (t, J=5 Hz, 1H), 4.69 (br, 1H), 3.84-3.80 (m, 2H), 3.51-3.50 (m, 2H), 3.14-3.12 (br, 1H), 2.48 (br, 1H), 2.32-2.28 (br, 1H), 1.95-1.59 (m, 20H). ¹³C NMR (125 MHz, CDCl₃), S 156.8, 135.21 (d, ⁴J_{C, P}=2.5 Hz), 133.70 (d, ³J_{C, P}=10.08 Hz), 130.66 (d, ²J_{C, P}=12.6 Hz), 118.45 (d, ¹J_{C, P}=85.7 Hz), 110.54, 107.31, 70.38, 40.46 (d, J_{C,P}=17.6 Hz), 37.08, 34.38, 33.26, 30.19, 29.82, 28.36, 27.18, 26.7, 25.79, 23.04, 20.86 (d, J_{C,P}=51.7 Hz). ³¹P NMR (160 MHz, CDCl₃) δ 24.79. ESI-HRMS for C₃₈H₄₅NO₆P [M]⁺: calcd. 642.2979, found 642.2954.

(1r,3r,5r,7r)-dispiro[adamantane-2,3′-[1,2,4,5]tetraoxane-6′,1″-cyclohexan]-4″-yl(3-(dimethylamino)propyl)carbamate (30a): ¹H NMR (400 MHz, CDCl₃) δ 5.5 (brs, 1H), 4.8 (brs, 1H), 3.25-3.15 (m, 3H), 2.42-2.32 (m, 4H), 2.21 (s, 6H), 1.98-1.63 (m, 21H). ¹³C NMR (100 MHz, CDCl₃) δ 156.22, 110.68, 107.34, 69.93, 58.14, 45.58, 40.33, 37.05, 34.4, 33.26, 30.21, 28.19, 27.17, 27.16, 26.62, 25.65. ESI-HRMS for C₂₂H₃₇N₂O₆[M+H]⁺: calcd. 425.2573, found 425.2618.

3-((1r,3r,5r,7r)-6′-methylspiro[adamantane-2,3′-[1,2,4,5]tetraoxan]-6′-yl)propanoic acid (35): ¹H NMR (500 MHz, CDCl₃) δ 10.64 (br, 1H), 3.11 (br, 1H), 2.61-2.49 (m, 4H), 1.95-1.61 (m, 14H), 1.27 (br, 2H). ¹³C NMR (125 MHz, CDCl₃) δ 179.43, 110.37, 108.43, 37.08, 34.34, 33.26, 33.23, 30.31, 29.26, 27.77, 27.19, 20.06. ESI-HRMS for C₁₅H₂₂NaO₆ [M+Na]⁺: calcd. 321.1314, found 321.1288.

N-(3-(dimethylamino)propyl)-3-((1r,3r,5r,7r)-6′-methylspiro[adamantane-2,3′-[1,2,4,5]tetraoxan]-6′-yl)propenamide (35a): ¹H (500 MHz, CD₃OD) δ 3.23 (t, J=10 Hz, 2H), 3.16-3.11 (br, 1H), 2.64 (t, J=10 Hz, 2H), 2.53-2.48 (m, 7H), 2.33-2.3 (m, 2H), 1.97-1.67 (m, 16H), 1.25 (br, 2H). ¹³C (150 MHz, CD₃OD) δ 175.6, 110.02, 110.00, 57.54, 44.7, 37.93, 35.55, 34.11, 34.08, 33.36, 31.91, 31.47, 29.71, 28.53, 27.42, 20.17. ESI-HRMS for C₂₀H₃₅N₂O₅[M+H]⁺: calcd. 383.2468, found 383.2516.

12-(tert-butyl)-7,8,15,16-tetraoxadispiro[5.2.59.26]hexadecan-3-ol (37): ¹H NMR (400 MHz, CDCl₃, 298 K) δ 3.85-3.83 (m, 1H), 3.13 (br, 1H), 2.62 (br, 1H), 2.12 (br, 1H), 1.88-1.4 (m, 12H), 1.25-1.23 (m, 2H), 1.06-1.03 (m, 1H), 0.84 (s, 9H). ¹H NMR (400 MHz, CDCl₃, 320 K) δ 3.85-3.83 (m, 1H), 3.10 (br, 1H), 2.44-1.62 (m, 12H), 1.49-1.40 (tt, J=13.5, 3.9 Hz, 2H), 1.32-1.22 (m, 2H), 1.10-1.04 (m, 1H), 0.86 (s, 9H). ¹H NMR (500 MHz, CDCl₃, 258 K) δ 3.90 (s, 1H), 3.86 (s, 1H), 3.15 (s, 1H), 3.13 (s, 1H), 2.70 (s, 1H), 2.60 (s, 1H), 2.19 (s, 1H), 2.07 (s, 1H), 1.84-1.73 (m, 10H), 1.66-1.54 (m, 10H), 1.48-1.43 (m, 4H), 1.25-1.19 (m, 4H), 1.06 (t, J=12.0 Hz, 2H), 0.83 (s, 18H). ¹³C NMR (100 MHz, CDCl₃, 298 K) δ 108.33, 107.49 (dr), 107.46 (dr), 67.76 (dr), 67.52 (dr), 47.39 (dr), 47.36 (dr), 32.3, 31.97, 30.2, 29.56, 28.2, 27.57, 25.55, 23.13, 22.66. ¹³C NMR (100 MHz, CDCl₃, 320 K) δ 108.45, 107.64 (dr), 107.61 (dr), 67.91 (dr), 67.74 (dr), 47.70 (dr), 47.68 (dr), 32.44, 30.08, 27.71, 23.10. ¹³C NMR (125 MHz, CDCl₃, 258 K) δ 108.51, 107.60, 107.53, 68.00, 67.49, 47.15, 47.11, 32.43, 31.84, 30.26, 30.05, 29.45, 29.24, 28.37, 27.97, 27.64, 25.67, 25.27, 23.09, 22.53. ESI-HRMS for C16H29O₅ [M+H]⁺: calcd. 301.1937, found 301.1393 or ESI-HRMS for C16H28NaO₅ [M+Na]⁺: calcd. 323.1834, found 323.1810.

12-(tert-butyl)-7,8,15,16-tetraoxadispiro[5.2.59.26]hexadecan-3-yl(4-nitrophenyl) carbonate (37c): ¹H NMR (500 MHz, CDCl₃) δ 8.28 (d, J=9.1 Hz, 2H), 7.39 (d, J=9.1 Hz, 2H), 4.95-4.93 (m, 1H), 3.16 (br, 1H), 2.51-2.45 (m, 2H), 1.96-1.61 (m, 10H), 1.51-1.45 (m, 3H), 1.12-1.07 (m, 1H), 0.87 (s, 9H). ¹³C NMR (125 MHz, CDCl₃), δ 155.7, 152, 145.59, 125.43, 121.88, 108.75, 106.99 (dr), 106.94 (dr), 75.9 (dr), 75.73 (dr), 47.6, 32.47, 32.11, 31.71, 29.83, 27.72, 26.27, 25.34, 23.11, 22.78. ESI-HRMS for C23H31NNaO₉ [M+Na]⁺: calcd. 488.1897, found 488.1867.

12-(tert-butyl)-7,8,15,16-tetraoxadispiro[5.2.59.26]hexadecan-3-yl(3-(dimethylamino)propyl)carbamate (37a): ¹H NMR (500 MHz, CDCl₃, 320 K) δ 5.38 (br, 1H), 4.82 (br, 1H), 3.25 (q, J=5.7 Hz, 2H), 2.40-2.25 (m, 10H), 1.83-1.67 (m, 9H), 1.47 (td, J=13.6, 3.7 Hz, 2H), 1.32-1.27 (m, 4H), 1.09 (t, J=11.6 Hz, 1H), 0.86 (s, 9H). ¹³C NMR (125 MHz, CDCl₃, 320 K) δ 156.27, 108.58, 107.55 (dr), 107.52 (dr), 70.25 (dr), 70.11 (dr), 58.01, 47.79 (dr), 47.77 (dr), 45.4, 40.29, 32.51, 29.85, 27.76, 27.33, 26.98, 23.17. ESI-HRMS calcd. for C₂₂H₄₁N₂O₆[M+H]⁺: calcd. 429.2886, found 429.2934.

(3-((((12-(tert-butyl)-7,8,15,16-tetraoxadispiro[5.2.59.26]hexadecan-3-yl)oxy)carbonyl)amino)propyl)triphenylphosphonium bromide (37b): ¹H NMR (600 MHz, CDCl₃) δ 7.8-7.74 (m, 9H), 7.69-7.66 (m, 6H), 6.8-6.72 (dt, J=38.7, 6.1 Hz, 1H), 4.71-4.68 (m, 1H), 3.79-3.75 (m, 2H), 3.49 (brs, 2H), 3.15-3.13 (br, 1H), 2.5-2.24 (m, 2H), 1.84-1.68 (m, 10H), 1.55 (br, 2H), 1.45-1.43 (m, 2H), 1.07-1.06 (m, 1H), 0.85 (d, 9H). ¹³C NMR (150 MHz, CDCl₃) δ 156.79, 135.23 (d, ⁴J_{C, P}=3 Hz), 133.64 (d, ³J_{C, P} 10.6 Hz), 130.64 (d, 2J_{C, P}=12.1 Hz), 118.34 (d, ¹J_{C, P}=86.1 Hz), 108.41, 107.53 (dr), 107.43 (dr), 70.4 (dr), 70.1 (dr), 47.53 (dr), 47.45 (dr), 40.45 (d, ²J_{C, P}=18 Hz), 32.45, 32.05, 29.82, 28.35, 28.11, 27.7, 27.20, 26.58, 25.66, 25.41, 23.26, 22.94, 22.73, 20.56 (d, ¹J_{C,P}=52.9 Hz). ³¹P NMR (160 MHz, CDCl₃) δ 24.70. ESI-HRMS for C38H49NO₆P [M]⁺: calcd. 646.3292, found 646.3261.

N₁,N₁-dibenzylpropane-1,3-diamine (72): ¹H NMR (400 MHz, CDCl₃) δ 7.33-7.27 (m, 8H), 7.24-7.19 (m, 2H), 3.64 (brs, 2H), 3.53 (s, 4H), 2.71 (t, J=6.5 Hz, 2H), 2.45 (t, J=6.4 Hz, 2H), 1.70-1.68 (m, 2H). ¹³C NMR (100 MHz, CDCl₃) δ 139.22, 128.87, 128.28, 126.96, 58.31, 50.63, 39.79, 28.74.

12-(tert-butyl)-7,8,15,16-tetraoxadispiro[5.2.59.26]hexadecan-3-yl(3-(dibenzylamino)propyl)carbamate (37d): ¹H NMR (600 MHz, CDCl₃) δ 7.33 (br, 10H), 5.23-5.21 (m, 1H), 4.79 (m, 1H), 3.51 (brs, 5H), 3.17-3.11 (m, 3H), 2.48-2.41 (m, 4H), 1.79-1.63 (m, 10H), 1.48 (m, 2H), 1.11-1.07 (m, 1H), 0.87 (s, 9H). ¹³C NMR (150 MHz, CDCl₃), δ 139.40, 129.14, 128.53, 127.53, 108.57, 107.60 (dr), 107.52 (dr), 69.69 (dr), 69.39 (dr), 58.79 (CH₂), 51.26 (CH₂), 47.58, 39.6 (CH₂), 32.49, 32.15, 29.85, 27.74, 27.11, 26.56, 26.28, 25.51, 25.34, 23.33, 22.84. ESI-HRMS for C₃₄H₄₉N₂O₆ [M+H]⁺: calcd. 581.3512, found 581.3558.

N1-benzyl-N1-methylpropane-1,3-diamine (73): ¹H NMR (400 MHz, CDCl₃) δ 7.30 (d, J=4.4 Hz, 4H), 7.23 (dq, J=8.7, 4.2 Hz, 1H), 3.46 (s, 2H), 2.73 (t, J=6.8 Hz, 2H), 2.40 (t, J=7.0 Hz, 2H), 2.18 (s, 3H), 1.64 (p, J=6.9 Hz, 2H). ¹³C NMR (100 MHz, CDCl₃) δ 139.18, 128.89, 128.14, 126.84, 62.48, 54.99, 42.20, 40.50, 31.05.

12-(tert-butyl)-7,8,15,16-tetraoxadispiro[5.2.59.26]hexadecan-3-yl(3-(benzyl(methyl)amino)propyl)carbamate (37e): ¹H NMR (500 MHz, CDCl₃) δ 7.32-7.28 (m, 5H), 5.75 (br, 1H), 4.8 (br, 1H), 3.48 (s, 2H), 3.24-3.17 (m, 3H), 2.45-2.44 (m, 4H), 2.19 (s, 3H), 1.75-1.68 (m, 12H), 1.50-1.44 (m, 2H), 1.11-1.07 (m, 1H), 0.87 (s, 9H). ¹³C NMR (100 MHz, CDCl₃) δ 156.19, 138.93, 129.12, 128.5, 127.34, 108.54, 107.56 (dr), 107.5 (dr), 69.88 (dr), 69.6 (dr), 62.83, 56.01, 55.93, 47.56, 42.23, 40.52, 32.47, 32.07, 29.84, 28.30, 27.73, 27.13, 26.42, 25.66, 25.41, 23.31, 22.83. ESI-HRMS for C₂₈H₄₅N₂O₆ [M+H]⁺: calcd. 505.3199, found 505.3263.

12-(tert-butyl)-7,8,15,16-tetraoxadispiro[5.2.59.26]hexadecan-3-yl piperazine-1-carboxylate (37f): ¹H NMR (400 MHz, CDCl₃) δ 4.86-4.84 (m, 1H), 3.43 (brs, 5H), 3.15 (br, 1H), 2.82 (brs, 4H), 2.4-2.31 (m, 2H), 2.10-2.08 (m, 1H), 1.83-1.63 (m, 10H), 1.48-1.44 (m, 2H), 1.1-1.06 (m, 1H), 0.86 (s, 9H). ¹³C NMR (100 MHz, CDCl₃) δ 154.86, 108.59, 107.46 (dr), 107.42 (dr), 70.62 (dr), 70.41 (dr), 47.54, 47.48, 45.88, 45.04, 44.65, 32.45, 32.05, 29.83, 28.24, 27.7, 27.11, 26.49, 25.61, 23.3, 22.76. ESI-HRMS for C₂₁H₃₇N₂O₆[M+H]⁺: calcd. 413.2573, found 413.2631.

3-(2,4-dimethyl-3,6-dioxocyclohexa-1,4-dien-1-yl)-3-methylbutanoic acid (43): ¹H NMR (300 MHz, CDCl₃) δ 6.44 (s, 1H), 3.03 (s, 2H), 2.16 (s, 3H), 1.97 (s, 3H), 1.43 (s, 6H). ¹³C NMR (75 MHz, CDCl₃) δ 189.80, 188.37, 178.34, 151.00, 143.86, 140.50, 135.00, 47.47, 38.31, 29.26, 15.61, 14.64.

12-(tert-butyl)-7,8,15,16-tetraoxadispiro[5.2.59.26]hexadecan-3-yl 4-(3-(2,4-dimethyl-3,6-dioxocyclohexa-1,4-dien-1-yl)-3-methylbutanoyl)piperazine-1-carboxylate (37 g): ¹H NMR (400 MHz, CDCl₃) δ 6.42 (s, 1H), 4.86 (br, 1H), 3.5-3.38 (m, 8H), 3.04 (s, 2H), 2.39 (br, 1H), 2.16 (s, 3H), 1.98 (s, 3H), 1.85-1.6 (m, 7H), 1.48-1.44 (m, 8H), 1.11-1.07 (m, 1H), 0.86 (s, 9H). ¹³C NMR (100 MHz, CDCl₃) δ 190.21, 188.42, 170.78, 154.74, 153.32, 143.87, 138.14, 134.99, 108.65, 107.33 (dr), 107.29 (dr), 71.25 (dr), 71.02 (dr), 47.54, 47.49, 47.34, 45.44, 43.69, 41.3, 38.22, 32.46, 32.06, 29.83, 29.25, 28.3, 27.71, 27.11, 26.45, 25.71, 23.29, 22.78, 15.78, 14.64. ESI-HRMS for C₃₄H₅₁N₂O₉[M+H]⁺: calcd. 631.3516, found 631.3566.

4-(dibenzylamino)butan-1-ol (44): ¹H NMR (400 MHz, CDCl₃) δ 7.3-7.20 (m, 10H), 4.60 (s, 2H), 3.54 (s, 4H), 3.49 (t, J=5.8 Hz, 2H), 2.42 (t, J=6.2 Hz, 2H), 1.62-1.57 (m, 2H), 1.54-1.49 (m, 2H). ¹³C NMR (100 MHz, CDCl₃) δ 141.19, 138.39, 129.31, 128.47, 128.31, 127.43, 127.14, 126.93, 64.96, 62.51, 58.26, 53.65, 31.25, 24.48. ESI-HRMS for C₁₈H₂₄NO [M+H]⁺: calcd. 270.1780, found 270.1851.

(2R,3R,4S,5R,6R)-2-(acetoxymethyl)-6-(4-aminobutoxy)tetrahydro-2H-pyran-3,4,5-triyl triacetate (46): ¹H NMR (500 MHz, CDCl₃) δ 5.19 (t, J=9.5 Hz, 1H), 5.06 (t, J=9.7 Hz, 1H), 4.94 (t, J=9.5 Hz, 1H), 4.50 (d, J=8.0 Hz, 1H), 4.24 (dd, J=12.3, 4.5 Hz, 1H), 4.15 (d, J=12.3 Hz, 1H), 3.89-3.85 (m, 1H), 3.71-3.69 (m, 1H), 3.56-3.53 (m, 1H), 3.02 (t, J=6.9 Hz, 2H), 2.09 (s, 3H), 2.07 (s, 3H), 2.01 (s, 3H), 1.99 (s, 3H), 1.82-1.80 (m, 2H), 1.72-1.71 (m, 2H). ¹³C NMR (125 MHz, CDCl₃) δ 100.77, 72.85, 71.97, 71.41, 69.20, 68.51, 61.99, 39.69, 26.50, 24.46, 20.96, 20.72. ESI-HRMS for C₁₈H₃₀NO₁₀ [M+H]⁺: calcd. 420.1791, found 420.1860.

(2R,3R,4S,5R,6R)-2-(acetoxymethyl)-6-(4-((((12-(tert-butyl)-7,8,15,16-tetraoxa dispiro[5.2.59.26]hexadecan-3-yl)oxy)carbonyl)amino)butoxy)tetrahydro-2H-pyran-3,4,5-triyl triacetate (37 h): ¹H NMR (500 MHz, CDCl₃) δ 5.19 (t, J=9.5 Hz, 1H), 5.08 (t, J=9.7 Hz, 1H), 4.97 (dd, J=9.6, 8.0 Hz, 1H), 4.78 (br, 1H), 4.48 (d, J=7.9 Hz, 1H), 4.25 (dd, J=12.3, 4.7 Hz, 1H), 4.16-4.09 (m, 1H), 3.89-3.86 (m, 1H), 3.68 (ddd, J=9.9, 4.5, 2.3 Hz, 1H), 3.52-3.49 (m, 1H), 3.18-3.17 (m, 2H), 2.40-2.33 (m, 2H), 2.08 (s, 3H), 2.04-2.02 (m, 9H), 1.80-1.54 (m, 14H), 1.46 (t, J=13.5 Hz, 2H), 1.31-1.23 (m, 2H), 1.10-1.05 (m, 1H), 0.86 (s, 9H). ¹³C NMR (125 MHz, CDCl₃) δ 170.82, 170.43, 169.55, 169.49, 156.13, 108.56, 107.47 (dr), 107.42 (dr), 100.96, 72.96, 71.97, 71.49, 70.22 (dr), 69.98 (dr), 69.73, 68.61, 62.07, 47.58, 40.52, 32.46, 32.16, 29.83, 27.72, 27.01, 26.67, 25.53, 23.28, 22.83, 20.88, 20.80, 20.75, 20.73. ESI-HRMS for C₃₅H₅₆NO₁₆ [M+H]⁺: calcd. 746.3521, found 746.3582.

12-(tert-butyl)-7,8,15,16-tetraoxadispiro[5.2.59.26]hexadecan-3-yl(4-(((2R,3R,4S,5S,6R)-3,4,5-trihydroxy-6-(hydroxymethyl)tetrahydro-2H-pyran-2-yl)oxy)butyl)carbamate (37i): ¹H NMR (500 MHz, Acetone-d₆) δ 6.35 (s, 1H), 4.74 (s, 1H), 4.26 (d, J=7.7 Hz, 1H), 3.88-3.85 (m, 1H), 3.82 (dd, J=11.7, 2.0 Hz, 1H), 3.63 (dd, J=11.7, 5.2 Hz, 1H), 3.53-3.51 (m, 1H), 3.37-3.26 (m, 3H), 3.15 (t, J=8.3 Hz, 3H), 2.36 (br, 2H), 1.76-1.59 (m, 13H), 1.48-1.43 (m, 2H), 1.28-1.15 (m, 4H), 0.87 (s, 9H). ¹³C NMR (150 MHz, Acetone-d₆) δ 156.91, 108.81, 107.97, 104.02, 77.80, 77.42, 74.75, 71.52, 70.09 (dr), 69.97 (dr), 69.55, 62.80, 47.97, 41.03, 40.90, 32.76, 32.49, 28.71, 27.81, 27.44, 27.33, 27.19, 26.13, 26.05, 23.85, 23.34. ESI-HRMS for C₂₇H₄₈NO₁₂ [M+H]⁺: calcd. 578.3098, found 578.3158.

bis(12-(tert-butyl)-7,8,15,16-tetraoxadispiro[5.2.59.26]hexadecan-3-yl) ((ethane-1,2-diylbis(oxy))bis(ethane-2,1-diyl))dicarbamate (37j): ¹H NMR (400 MHz, CDCl₃) δ 5.19 (br, 1H), 4.82 (br, 1H), 3.62 (s, 4H), 3.57-3.55 (m, 4H), 3.38-3.33 (m, 4H), 3.15 (br, 1H), 2.44-2.33 (m, 3H), 1.77-1.63 (m, 21H), 1.48-1.44 (m, 4H), 1.08-1.06 (m, 2H), 0.86 (s, 18H). ¹³C NMR (100 MHz, CDCl₃) δ 156.13, 108.56, 107.45 (dr), 107.39 (dr), 70.48 (CH₂), 70.37 (dr), 70.25 (dr), 47.55, 40.83 (CH₂), 32.47, 32.07, 29.84, 28.11, 27.72, 27.12, 26.53, 25.65, 25.47, 23.3, 22.83. ESI-HRMS for C₄₀H₆₉N₂O₁₄ [M+H]⁺: calcd. 801.4671, found 801.4714.

bis(12-(tert-butyl)-7,8,15,16-tetraoxadispiro[5.2.59.26]hexadecan-3-yl)methyl phosphate (37k): ¹H NMR (500 MHz, CDCl₃) δ 4.55 (br, 2H), 3.75 (d, J=15 Hz, 3H), 3.15 (br, 2H), 2.50-2.33 (m, 4H), 1.88-1.58 (m, 20H), 1.49-1.44 (m, 4H), 1.11-1.06 (m, 2H), 0.86 (s, 18H). ¹³C NMR (125 MHz, CDCl₃) δ 108.67, 107.14 (dr), 107.08 (dr), 74.57 (d, ¹J_{C-p}=5.04 Hz) (dr), 74.39 (d, ¹J_{C-p}=6.3 Hz) (dr), 54.29 (d, ¹J_{C-p}=6.3 Hz), 32.47, 32.08, 29.85, 28.43, 27.72, 25.08, 23.29, 22.78. ³¹P NMR (160 MHz, CDCl₃) δ−1.22. ESI-HRMS calcd. for C₃₃H₅₈O₁₂P [M+H]⁺: calcd. 676.3588, found 676.3638.

2,2′-disulfanediylbis(ethan-1-ol) (47): ¹H NMR (500 MHz, CDCl₃) δ 3.85 (q, J=5 Hz, 4H), 2.84 (q, J=6 Hz, 4H). ¹³C NMR (125 MHz, CDCl₃) δ 60.42, 41.25.

bis(12-(tert-butyl)-7,8,15,16-tetraoxadispiro[5.2.59.26]hexadecan-3-yl) (disulfanediylbis(ethane-2,1-diyl))bis(carbonate) (37l): ¹H NMR (600 MHz, CDCl₃) δ 4.82-4.79 (m, 2H), 4.38 (t, J=6 Hz, 4H), 3.15 (br, 2H), 2.97 (t, J=6 Hz, 4H), 2.45-2.35 (m, 4H), 1.88-1.57 (m, 22H), 1.49-1.44 (m, 4H), 1.10-1.06 (m, 2H), 0.86 (s, 18H). ¹³C NMR (150 MHz, CDCl₃) δ 154.48, 108.64, 107.20 (dr), 107.14 (dr), 74.34 (dr), 74.27 (dr), 74.13 (dr), 74.05 (dr), 65.62, 47.56, 47.53, 37.14, 32.47, 32.07, 29.84, 27.72, 26.73, 26.17, 25.47, 25.22, 23.27, 22.83. ESI-HRMS for C₃₈H₆₂NaO₁₄S₂[M+Na]⁺: calcd. 829.3479 [M+H]⁺, found 829.3874.

N,N-dibenzyl-3-((((12-(tert-butyl)-7,8,15,16-tetraoxadispiro[5.2.59.26]hexadeca n-3-yl)oxy)carbonyl)amino)-N-methylpropan-1-aminium iodide (48): ¹H NMR (600 MHz, CDCl₃) δ 7.60 (d, 4H), 7.50-7.43 (m, 6H), 5.50 (dt, J=25.4, 6 Hz, 1H), 4.93-4.88 (br, 4H), 4.75 (br, 1H), 3.43-3.40 (m, 2H), 3.29 (q, J=6 Hz, 2H), 3.16 (br, 1H), 3.04 (s, 3H), 2.45-2.30 (m, 4H), 1.78-1.64 (m, 11H), 1.46 (t, J=12 Hz, 2H), 1.07 (t, J=12 Hz, 1H), 0.86 (s, 9H). ¹³C NMR (150 MHz, CDCl₃) δ 156.62, 133.37, 131.17, 129.62, 126.71, 108.58, 107.39 (dr), 107.31 (dr), 70.80 (dr), 70.51 (dr), 65.25, 57.96, 47.53, 46.88, 38.02, 32.47, 32.06, 29.84, 27.72, 27.08, 26.52, 25.66, 25.46, 24.30, 23.32, 22.83. ESI-HRMS for C₃₅H₅₁N₂O₆, [M]⁺: calcd. 595.3742, found 595.3722.

tributyl(3-((((12-(tert-butyl)-7,8,15,16-tetraoxadispiro[5.2.59.26]hexadecan-3-yl)oxy)carbonyl)amino)propyl)phosphonium bromide (56): ¹H NMR (500 MHz, CDCl₃) δ 6.08-5.99 (m, 1H), 4.75 (br, 1H), 3.33 (q, J=11.4 Hz, 6H), 3.14 (br, 1H), 2.56-2.29 (m, 11H), 1.90-1.52 (m, 24H), 1.44-1.42 (m, 2H), 1.09-1.05 (m, 1H), 0.97 (t, J=6.7 Hz, 9H), 0.85 (s, 9H). ¹³C NMR (125 MHz, CDCl₃) δ 156.63, 108.37, 107.32 (dr), 107.23 (dr), 70.45 (dr), 70.16 (dr), 47.42, 40.95 (d, ³J_{c, p}=16.4 Hz), 32.32, 31.95, 29.70, 27.58, 27.03, 26.47, 25.51, 23.97 (d, 2J_{c, p}=15.1 Hz), 23.71 (d, ³J_{c, p}=5 Hz), 23.12, 22.69, 22.05, 19.00 (d, ¹J_{c, p}=47.9 Hz), 16.88 (d, J_{c, p}=49.1 Hz), 13.45. ³¹P NMR (160 MHz, CDCl₃) δ 33.88. ESI-HRMS for C₃₂H₆₁NO₆P [M]⁺: calcd. 586.4231, found 586.4212.

4-(((((12-(tert-butyl)-7,8,15,16-tetraoxadispiro[5.2.59.26]hexadecan-3-yl)oxy) carbonyl)amino)methyl)-1-methylpyridin-1-ium iodide (50): ¹H NMR (500 MHz, CDCl₃) δ 8.95 (s, 2H), 8.06 (s, 2H), 6.48-6.39 (m, 1H), 4.83-4.38 (m, 6H), 3.13-2.84 (m, 4H), 2.33-2.17 (m, 2H), 2.04-1.46 (m, 10H), 1.08 (m, 1H), 0.86 (s, 9H). ¹³C NMR (125 MHz, CDCl₃) δ 160.44, 156.61, 144.94, 126.71, 108.61, 107.83, 107.45 (dr), 107.35 (dr), 71.61 (dr), 71.20 (dr), 49.40, 47.54, 44.21, 32.47, 32.07, 29.84, 29.17, 27.73, 27.06, 25.53, 23.80, 23.44, 22.83. ESI-HRMS for C₂₄H₃₇N₂O₆, [M]⁺: calcd. 449.2646, found 449.2627.

4-(((12-(tert-butyl)-7,8,15,16-tetraoxadispiro[5.2.59.26]hexadecan-3-yl)oxy)carbonyl)-1,1-dimethylpiperazin-1-ium iodide (51): ¹H NMR (500 MHz, CDCl₃) δ 4.85 (br, 1H), 3.89-3.64 (m, 13H), 3.13 (br, 1H), 2.44-2.21 (m, 3H), 1.96-1.81 (m, 10H), 1.48-1.42 (m, 2H), 1.11-1.05 (m, 1H), 0.86 (s, 9H). ¹³C NMR (125 MHz, CDCl₃) δ 154.03, 108.67, 107.85, 107.22 (dr), 107.12 (dr), 72.75 (dr), 72.40 (dr), 61.68, 52.37, 47.53 (dr), 47.51 (dr), 38.12, 32.45, 32.05, 29.82, 29.14, 27.71, 27.14, 26.45, 25.83, 23.77, 23.43, 22.81. ESI-HRMS for C₂₃H₄₁N₂O₆ [M]⁺: calcd. 441.2959, found 441.2946.

3-((((12-(tert-butyl)-7,8,15,16-tetraoxadispiro[5.2.59.26]hexadecan-3-yl)oxy)carbonyl)amino)-N,N,N-trimethylpropan-1-aminium iodide (49): ¹H NMR (500 MHz, CDCl₃) δ 5.88-5.77 (m, 1H), 4.78 (br, 1H), 3.75 (br, 2H), 3.41-3.33 (m, 10H), 3.14 (br, 1H), 2.44-1.60 (m, 16H), 1.47-1.42 (m, 2H), 1.10-1.05 (m, 1H), 0.86 (s, 9H). ¹³C NMR (125 MHz, CDCl₃) δ 156.46, 108.49, 108.44, 107.44 (dr), 107.32 (dr), 70.39 (dr), 69.98 (dr), 65.09, 54.06, 47.41 (dr), 47.38 (dr), 37.74, 32.33, 31.93, 29.70, 29.02, 27.58, 26.93, 26.40, 25.35, 23.92, 23.85, 23.18, 22.70. ESI-HRMS calcd. for C₂₃H₄₃N₂O₆ [M]⁺: calcd. 443.3116, found 443.3093.

N₁,N₁-dibutylpropane-1,3-diamine (52): ¹H NMR (500 MHz, CDCl₃) δ 2.68 (t, J=6.8 Hz, 2H), 2.41-2.39 (m, 2H), 2.35-2.32 (m, 4H), 1.53 (p, J=5 Hz, 2H), 1.41-1.33 (m, 6H), 1.28-1.21 (m, 4H), 0.86 (t, J=7.3 Hz, 6H). ¹³C NMR (125 MHz, CDCl₃) δ 54.00, 52.09, 41.01, 31.07, 29.32, 20.82, 14.16.

N-butyl-N-(3-((((12-(tert-butyl)-7,8,15,16-tetraoxadispiro[5.2.59.26]hexadecan-3-yl)oxy)carbonyl)amino)propyl)-N-methylbutan-1-aminium iodide (53): ¹H NMR (500 MHz, CDCl₃) δ 6.04-5.89 (brs, 1H), 4.76-4.73 (br, 1H), 3.56-3.53 (m, 2H), 3.39-3.31 (m, 6H), 3.21 (s, 3H), 3.11 (br, 1H), 2.41-2.29 (m, 2H), 2.05-2.00 (m, 3H), 1.76-1.56 (m, 12H), 1.46-1.37 (m, 6H), 1.05 (t, J=10 Hz, 1H), 0.97 (t, J=5 Hz, 6H), 0.83 (s, 9H). ¹³C NMR (125 MHz, CDCl₃) δ 156.51, 108.46, 108.42, 107.43 (dr), 107.30 (dr), 70.39 (dr), 69.95 (dr), 62.08, 60.17, 49.33, 47.45, 37.76, 32.36, 32.05, 29.72, 27.62, 27.53, 26.99, 26.47, 24.45, 23.31, 23.27, 22.72, 19.75, 13.78. ESI-HRMS for C₂₉H₅₅N₂O₆ [M]⁺: calcd. 527.4055, found 527.4031.

N-benzyl-3-((((12-(tert-butyl)-7,8,15,16-tetraoxadispiro[5.2.59.26]hexadecan-3-yl)oxy)carbonyl)amino)-N,N-dimethylpropan-1-aminium iodide (54): ¹H NMR (500 MHz, CDCl₃) δ 7.6-7.49 (m, 5H), 5.67-5.61 (m, 1H), 4.79 (s, 2H), 3.70 (br, 2H), 3.35 (br, 2H), 3.22-3.12 (m, 6H), 2.34-2.02 (m, 4H), 1.80-1.61 (m, 15H), 1.46 (t, J=12.1 Hz, 2H), 1.08 (t, J=15 Hz, 1H), 0.86 (s, 9H). ¹³C NMR (125 MHz, CDCl₃) δ 156.69, 133.23, 131.43, 129.74, 128.77, 127.49, 126.51, 108.59, 107.45 (dr), 107.37 (dr), 70.80 (dr), 70.51 (dr), 68.55, 62.74, 50.37, 47.56, 38.07, 32.47, 32.07, 29.85, 29.51, 27.73, 27.04, 26.62, 23.98, 23.95, 23.28, 22.84. ESI-HRMS for C₂₉H₄₇N₂O₆ [M]⁺: calcd. 519.3429, found 519.3403.

N,N,N-tribenzyl-3-((((12-(tert-butyl)-7,8,15,16-tetraoxadispiro[5.2.59.26]hexadecan-3-yl)oxy)carbonyl)amino)propan-1-aminium iodide (57): ¹H NMR (500 MHz, CDCl₃) δ 7.64-7.62 (m, 6H), 7.56-7.52 (m, 9H), 5.22 (br, 1H), 4.72 (s, 7H), 3.36-3.34 (m, 2H), 3.19-3.18 (m, 3H), 2.34-2.31 (m, 3H), 2.04-2.00 (m, 3H), 1.79-1.76 (m, 9H), 1.49-1.44 (m, 2H), 1.11-1.06 (m, 1H), 0.87 (s, 9H). ¹³C NMR (125 MHz, CDCl₃) δ 156.65, 133.41, 131.49, 130.02, 126.78, 108.60, 107.84, 107.36 (dr), 107.30 (dr), 70.83 (dr), 70.60 (dr), 64.26, 58.15, 47.58 (dr), 47.56 (dr), 38.29, 32.47, 32.07, 29.84, 29.18, 27.73, 27.05, 26.62, 26.57, 25.50, 23.45, 23.28, 22.83. ESI-HRMS for C₄₁H₅₅N₂O₆ [M]⁺: calcd. 671.4055, found 671.4041.

7,8,15,16-tetraoxadispiro[5.2.59.26]hexadecan-3-ol (59): ¹H NMR (500 MHz, CDCl₃, 298 K) δ 3.84-3.83 (m, 1H), 2.62 (br, 1H), 2.26-2.13 (m, 3H), 1.8-1.45 (m, 15H). H NMR (500 MHz, CDCl₃, 320 K) δ 3.85-3.83 (m, 1H), 2.57-1.46 (m, 19H). ¹³C NMR (125 MHz, CDCl₃, 298 K) δ 108.49, 107.57, 67.81, 31.87, 30.37, 29.58, 28.37, 25.70, 25.45, 22.28, 21.99. ¹³C NMR (125 MHz, CDCl₃, 320 K) δ 108.50, 107.61, 67.86, 30.10, 25.52, 22.17. ESI-HRMS for C₁₂H₂₀O₅Na [M+Na]⁺: calcd. 267.1208, found 267.1200.

3-((((7,8,15,16-tetraoxadispiro[5.2.59.26]hexadecan-3-yl)oxy)carbonyl)amino)-N,N-dibenzyl-N-methylpropan-1-aminium iodide (59a): ¹H NMR (500 MHz, CDCl₃) δ 7.62 (d, J=7.1 Hz, 4H), 7.46-7.40 (m, 6H), 5.66 (t, J=6.1 Hz, 1H), 4.92 (s, 4H), 4.72 (br, 1H), 3.37-3.27 (m, 4H), 3.05 (s, 3H), 2.32 (br, 5H), 1.82-1.76 (m, 9H), 1.46 (br, 5H). ¹³C NMR (125 MHz, CDCl₃) δ 156.60, 133.42, 131.06, 129.56, 126.99, 108.58, 107.35, 70.62, 65.26, 58.03, 46.98, 38.07, 32.05, 30.96, 29.82, 29.47, 26.89, 25.80, 25.67, 25.50, 24.42, 22.94, 22.17. ESI-HRMS for C₃₁H₄₃N₂O₆ [M]⁺: calcd. 539.3116, found 539.3100.

(3-((((7,8,15,16-tetraoxadispiro[5.2.59.26]hexadecan-3-yl)oxy)carbonyl)amino) propyl)triphenylphosphonium bromide (59b): ¹H NMR (500 MHz, CDCl₃) δ 7.81-7.77 (m, 9H), 7.70-7.68 (m, 6H), 6.78 (br, 1H), 4.70 (br, 1H), 3.88-3.82 (m, 2H), 3.52-3.51 (m, 2H), 2.47 (br, 1H), 2.35-2.17 (m, 3H), 1.86-1.68 (m, 13H), 1.46-1.42 (m, 2H). ¹³C NMR (125 MHz, CDCl₃) δ 156.83, 135.22 (d, ⁴J_{C,P}=2.5 Hz), 133.72 (d, ³J_{C,P}=10 Hz), 130.66 (d, ²J_{C,P}=12.6 Hz), 118.48 (d, ¹J_{C,P}=86.9 Hz), 108.47, 107.49, 70.36, 40.46 (d, 2J_{C,P}=18.9 Hz), 31.92, 30.50, 29.84, 28.26, 27.17, 25.72, 25.51, 23.04, 22.32, 21.98, 20.88 (d, ¹J_{C,P}=51.7 Hz). ³¹P NMR (160 MHz, CDCl₃) δ 24.67 ppm. ESI-HRMS for C₃₄H₄₁NO₆P [M]⁺: calcd. 590.2666, found 590.2655.

Deferasirox (61): ¹H NMR (400 MHz, DMSO-d₆) δ 8.07 (d, J=7.6 Hz, 1H), 8.01 (d, J=8.3 Hz, 2H), 7.58-7.54 (m, 3H), 7.41-7.34 (m, 2H), 7.04-6.96 (m, 3H), 6.89 (d, J=8.2 Hz, 1H). ¹³C NMR (100 MHz, DMSO-d₆) δ 166.49, 160.01, 156.45, 155.21, 152.10, 141.31, 132.63, 131.53, 131.13, 130.58, 130.39, 126.86, 123.37, 119.76, 119.56, 117.12, 116.20, 114.48, 113.71.

N₁,N₁-dibenzyl-N3-(but-3-yn-1-yl)propane-1,3-diamine (alk-72): ¹H NMR (500 MHz, CDCl₃) δ 7.36-7.29 (m, 8H), 7.25-7.22 (m, 2H), 3.54 (s, 4H), 2.69 (t, J=6.8 Hz, 2H), 2.62 (t, J=6.8 Hz, 2H), 2.47 (t, J=6.7 Hz, 2H), 2.33 (td, J=6.8, 2.6 Hz, 2H), 1.96 (t, J=2.6 Hz, 1H), 1.74-1.70 (m, 2H). ¹³C NMR (125 MHz, CDCl₃) δ 139.80, 129.06, 128.37, 127.05, 79.30, 69.56, 58.60, 51.54, 48.18, 47.80, 29.85, 27.12, 19.61. ESI-HRMS for C₂₁H₂₇N₂[M+H]⁺: calcd. 307.2096, found 307.2164.

N,N-dibenzyl-3-(but-3-yn-1-yl(((12-(tert-butyl)-7,8,15,16-tetraoxadispiro[5.2.59.26]hexadecan-3-yl)oxy)carbonyl)amino)-N-methylpropan-1-aminium iodide (alk-R-48): ¹H NMR (500 MHz, CDCl₃, 320 K) δ 7.63 (d, J=5 Hz, 4H), 7.51-7.47 (m, 6H), 4.97 (brs, 4H), 4.82 (br, 1H), 3.47-3.42 (m, 6H), 3.09 (s, 3H), 2.45 (br, 2H), 2.33 (br, 2H), 2.03-1.54 (m, 17H), 1.12-1.08 (m, 1H), 0.88 (s, 9H). ¹³C NMR (125 MHz, CDCl₃, 320 K) δ 156.05, 133.44, 131.18, 129.65, 127.01, 108.73, 107.22, 87.07, 71.73 (dr), 70.78 (dr), 65.41, 57.88, 47.69, 47.10 (dr), 46.85 (dr), 45.69, 32.49, 32.08, 30.32, 29.85, 29.49, 27.74, 26.94, 23.13, 22.82. ESI-HRMS for C₃₉H₅₅N₂O₆ [M]⁺: calcd. 647.4055, found 647.4025.

(3-(but-3-yn-1-ylamino)propyl)triphenylphosphonium bromide (alk-68): ¹H NMR (500 MHz, CDCl₃, 320 K) δ 7.63 (d, J=5 Hz, 4H), 7.51-7.47 (m, 6H), 4.97 (brs, 4H), 4.82 (br, 1H), 3.47-3.42 (m, 6H), 3.09 (s, 3H), 2.45 (br, 2H), 2.33 (br, 2H), 2.03-1.54 (m, 17H), 1.12-1.08 (m, 1H), 0.88 (s, 9H). ¹³C NMR (125 MHz, CDCl₃, 320 K) δ 156.05, 133.44, 131.18, 129.65, 127.01, 108.73, 107.22, 87.07, 71.73 (dr), 70.78 (dr), 65.41, 57.88, 47.69, 47.10 (dr), 46.85 (dr), 45.69, 32.49, 32.08, 30.32, 29.85, 29.49, 27.74, 26.94, 23.13, 22.82. ESI-HRMS for C₃₉H₅₅N₂O₆ [M]⁺: calcd. 647.4055, found 647.4025.

(3-(but-3-yn-1-yl(((12-(tert-butyl)-7,8,15,16-tetraoxadispiro[5.2.59.26]hexadeca n-3-yl)oxy)carbonyl)amino)propyl)triphenylphosphonium bromide (alk-R-37b): ¹H NMR (500 MHz, CDCl3, 320 K) δ 7.86-7.77 (m, 9H), 7.70 (br, 6H), 4.76 (brs, 1H), 3.90 (brs, 2H), 3.64 (brs, 2H), 3.52 (brs, 2H), 2.48-2.33 (m, 4H), 2.16-2.03 (m, 3H), 1.84-1.55 (m, 12H), 1.12-1.10 (m, 1H), 0.88 (s, 9H). ¹³C NMR (126 MHz, CDCl₃, 320 K) δ 155.97, 135.17, 133.94 (d, ³J_{C,P}=10.2 Hz), 130.67 (d, 2J_{C,P}=12.6 Hz), 118.71 (d, ¹J_{C,P}=86.9 Hz), 108.63, 107.34, 81.99, 71.08 (dr), 70.93 (dr), 70.06, 48.35, 47.68, 32.50, 32.09, 29.85, 27.74, 26.94, 23.13, 22.83, 20.54 (d, J_{C,P}=51.7 Hz). ³¹P NMR (160 MHz, CDCl₃) δ 24.74 ppm. ESI-HRMS for C₄₂H₅₃NO₆P [M]⁺: calcd. 698.3605, found 698.3584.

2-methyl-2-(1,4-dioxaspiro[4.5]decan-8-yl)propan-1-ol (64): ¹H NMR (400 MHz, CDCl₃) δ 3.90 (s, 4H), 3.35 (s, 2H), 1.77-1.66 (m, 5H), 1.51-1.44 (m, 2H), 1.34-1.29 (m, 3H), 0.82 (s, 6H). ¹³C NMR (101 MHz, CDCl₃) δ 109.01, 70.72, 64.29, 64.25, 42.03, 37.08, 35.21, 24.46, 21.95. ESI-LRMS: m/z 215.25 [M+H]⁺; ESI-HRMS for C₁₂H₂₃O₃[M+H]⁺: calcd. 214.1569, found 215.1641.

4-(1-hydroxy-2-methylpropan-2-yl)cyclohexan-1-one (65): ¹H NMR (400 MHz, CDCl₃) δ 3.38 (s, 2H), 2.38-2.24 (m, 5H), 2.03 (ddd, J=13.2, 5.8, 3.0 Hz, 2H), 1.76 (tt, J=12.1, 3.0 Hz, 1H), 1.44 (qd, J=12.8, 5.1 Hz, 2H), 0.84 (s, 6H). ¹³C NMR (100 MHz, CDCl₃) δ 212.96, 70.48, 41.44, 41.34, 37.15, 27.27, 21.90.

of 2-methyl-2-(4-oxocyclohexyl)propyl but-3-yn-1-ylcarbamate (37): ¹H NMR (500 MHz, CDCl₃) δ 5.16 (br, 1H), 3.82 (s, 2H), 3.27 (q, J=5 Hz, 2H), 2.36-2.31 (m, 4H), 2.24 (td, J=14.1, 5.8 Hz, 2H), 2.00-1.96 (m, 3H), 1.65 (tt, J=12.1, 3.0 Hz, 1H), 1.41 (qd, J=12.9, 4.0 Hz, 2H), 0.85 (s, 6H). ¹³C NMR (125 MHz, CDCl₃) δ 212.15, 156.67, 81.59, 71.87, 70.18, 42.43, 41.36, 39.80, 36.31, 27.32, 22.36, 20.03. ESI-HRMS for C₁₅H₂₄NO₃ [M+H]⁺: calcd. 265.1678, found 266.1747.

2-(12-hydroxy-7,8,15,16-tetraoxadispiro[5.2.59.26]hexadecan-3-yl)-2-methylpropyl but-3-yn-1-ylcarbamate (alk-L-37): ¹H NMR (400 MHz, CDCl₃) δ 5.03 (s, 1H), 3.84 (brs, 3H), 3.31-3.15 (m, 3H), 2.61 (br, 1H), 2.39 (brs, 2H), 2.15-2.00 (m, 2H), 1.77-1.24 (m, 15H), 0.86 (s, 6H). ¹³C NMR (100 MHz, CDCl₃) δ 156.76, 108.19, 107.68, 81.58, 71.93, 70.14, 67.83 (dr), 67.57 (dr), 42.94, 39.74, 36.11, 31.91, 30.17, 29.62, 28.27, 25.69, 25.44, 22.85, 22.18, 19.98. ESI-HRMS for C₂₁H₃₃NNaO₇ [M+Na]⁺: calcd. 434.2155, found 434.2139.

N,N-dibenzyl-3-((((12-(1-((but-3-yn-1-ylcarbamoyl)oxy)-2-methylpropan-2-yl)-7,8,15,16-tetraoxadispiro[5.2.59.26]hexadecan-3-yl)oxy)carbonyl)amino)-N-methylpro pan-1-aminium iodide (alk-L-48): ¹H NMR (400 MHz, CDCl₃) δ 7.61 (d, J=4 Hz, 4H), 7.45-7.39 (m, 6H), 5.72-5.65 (m, 1H), 5.03-4.89 (m, 5H), 4.72 (br, 1H), 3.84 (s, 2H), 3.33-3.26 (m, 6H), 3.10-3.04 (m, 5H), 2.41-2.32 (m, 6H), 2.03-1.88 (m, 4H), 1.69-1.42 (m, 4H), 1.30-1.23 (m, 6H), 0.86 (s, 6H). ¹³C NMR (100 MHz, CDCl₃) δ 156.73, 156.57, 133.36, 130.89, 129.50, 126.87, 108.26, 107.45 (dr), 107.36 (dr), 81.56, 71.89, 70.55, 70.25 (dr), 70.13 (dr), 67.24, 65.08, 57.79, 46.89, 42.99 (dr), 42.90 (dr), 39.74, 37.92, 36.11, 31.92, 29.78, 29.59, 28.02, 27.02, 26.51, 25.36, 24.31, 22.81, 22.18, 19.99. ESI-HRMS for C₄₀H₅₆N₃O₈ [M]⁺: calcd. 706.4062, found 706.4031.

(3-((((12-(1-((but-3-yn-1-ylcarbamoyl)oxy)-2-methylpropan-2-yl)-7,8,15,16-tetr aoxadispiro[5.2.59.26]hexadecan-3-yl)oxy)carbonyl)amino)propyl)triphenylphosphonium bromide (alk-L-37b): ¹H NMR (500 MHz, CDCl₃) δ 7.79-7.68 (m, 15H), 6.83-6.78 (m, 1H), 5.02 (br, 1H), 4.70 (br, 1H), 3.84-3.80 (m, 4H), 3.49-3.48 (m, 2H), 3.33-3.32 (m, 2H), 3.15 (br, 1H), 2.47-2.23 (m, 4H), 2.03-1.77 (m, 11H), 1.54-1.43 (m, 3H), 1.30-1.24 (m, 4H), 0.86 (s, 6H). ¹³C NMR (125 MHz, CDCl₃) δ 156.78, 135.21 (d, ⁴J_{C,P}=2.5 Hz), 133.67 (d, ³J_{C,P}=10.1 Hz), 130.65 (d, ²J_{C,P}=12.6 Hz), 118.41 (d, ¹J_{C,P}=85.7 Hz), 108.15, 107.59 (dr), 107.51 (dr), 81.59, 71.92, 70.33 (dr), 70.12 (dr), 43.00 (dr), 42.89 (dr), 40.46 (d, ²J_{C,P}=17.6 Hz), 39.75, 36.13, 32.03, 29.80, 28.17, 27.08, 26.55, 25.67, 22.99, 22.79, 22.20, 20.74 (d, ¹J_{C,P}=51.7 Hz), 20.00. ³¹P NMR (160 MHz, CDCl₃) δ 24.70 ppm. ESI-HRMS for C₄₃H₅₄N₂O₈P [M]⁺: calcd. 757.3580, found 757.3580.

Methyl (E)-2-(1-(pyridin-2-yl)ethylidene)hydrazine-1-carbodithioate (74): E-isomer: ¹H NMR (400 MHz, CDCl₃) δ 10.06 (s, 0.59H), 8.59 (d, J=4.4 Hz, 0.59H), 8.18 (d, J=8.0 Hz, 0.59H), 7.72 (t, J=7.7 Hz, 0.59H), 7.32-7.28 (m, 0.59H), 2.67 (s, 1.8H), 2.45 (s, 1.8H). Z-isomer: ¹H NMR (400 MHz, CDCl₃) δ 8.74 (d, J=4.5 Hz, 0.41H), 7.90 (t, J=7.9 Hz, 0.41H), 7.59 (d, J=8.1 Hz, 0.41H), 7.40-7.38 (m, 0.41H), 2.64 (s, 0.82H), 2.44 (s, 0.82H). ¹³C NMR (100 MHz, CDCl₃) δ 201.80, 201.19, 154.35, 152.52, 149.99, 148.81 (E), 148.05 (Z), 140.57, 137.89 (Z), 136.47 (E), 124.48 (Z), 124.41 (E), 124.12 (Z), 120.98 (E), 22.22 (Z), 17.93 (E), 17.34 (Z), 11.26 (E).

N-(3-aminopropyl)-2-(1-(pyridin-2-yl)ethylidene)hydrazine-1-carbothioamide (75): E-isomer: ¹H NMR (400 MHz, CD₃OD) δ 8.57 (d, J=4 Hz, 0.69H), 8.18 (d, J=8.1 Hz, 0.69H), 7.83 (td, J=7.9, 1.7 Hz, 0.69H), 7.39 (dd, J=7.4, 4.9 Hz, 0.69H), 3.80 (t, J=6.7 Hz, 1.38H), 3.39 (t, J=4H, 0.69H), 2.72 (t, J=6.7 Hz, 1.38H), 2.39 (s, 2.1H), 1.84 (p, J=6.8 Hz, 1.38H). Z-isomer: ¹H NMR (400 MHz, CD₃OD) δ 8.45 (d, J=4.2 Hz, 0.19H), 8.07 (d, J=8.2 Hz, 0.18H), 7.76-7.72 (m, 0.19H), 7.25 (dd, J=6.8, 5.5 Hz, 0.19H), 2.31 (s, 0.56H), 2.00-1.92 (m, 0.38H). Thiol form: ¹H NMR (400 MHz, CD₃OD) δ 8.74 (d, J=4.0 Hz, 0.12H), 8.00 (td, J=7.9, 1.7 Hz, 0.12H), 7.49-7.46 (m, 0.12H), 7.48 (dd, J=7.6, 4.9 Hz, 0.12H), 2.00-1.92 (m, 0.24H). ¹³C NMR (100 MHz, CDCl₃) δ 178.35, 154.55, 148.93, 148.09, 136.54, 124.22, 120.89, 41.27, 29.48, 11.37. LRMS (EI, 20 eV) m/z 251 (M⁺, 5.37), 121(100); HRMS (EI) for C₁₁H₁₇N5S: calcd. 251.1205, found 251.1209.

N-(3-(((1r,3r,5r,7r)-dispiro[adamantane-2,3′-[1,2,4,5]tetraoxane-6′,1″-cyclohexan]-4″-yl)amino)propyl)-2-(1-(pyridin-2-yl)ethylidene)hydrazine-1-carbothioamide (77): ¹H NMR (500 MHz, DMSO-d₆) δ 10.33 (s, 1H), 8.81 (t, J=5.8 Hz, 1H), 8.56 (d, J=4.7 Hz, 1H), 8.36 (t, J=7.8 Hz, 1H), 8.22 (s, 1H), 7.82 (t, J=7.7 Hz, 1H), 7.40-7.37 (m, 1H), 3.37 (t, J=5.8 Hz, 2H), 3.08-2.88 (m, 4H), 2.38-2.33 (m, 3H), 1.98-1.88 (m, 5H), 1.79-1.60 (m, 15H), 1.47 (br, 2H). ¹³C NMR (150 MHz, CD₃OD) δ 180.24, 156.01, 149.81, 149.48, 138.33, 138.28, 125.40, 122.82, 111.67, 107.51, 56.94, 43.75, 41.84, 39.61, 39.56, 37.89, 35.62, 34.08, 34.06, 31.42, 30.34, 27.95, 26.40, 25.70, 22.25, 20.92. ESI-HRMS for C₂₇H₄₀N₅O₄S [M+H]⁺, calcd. 530.2723, found 530.2775.

Results

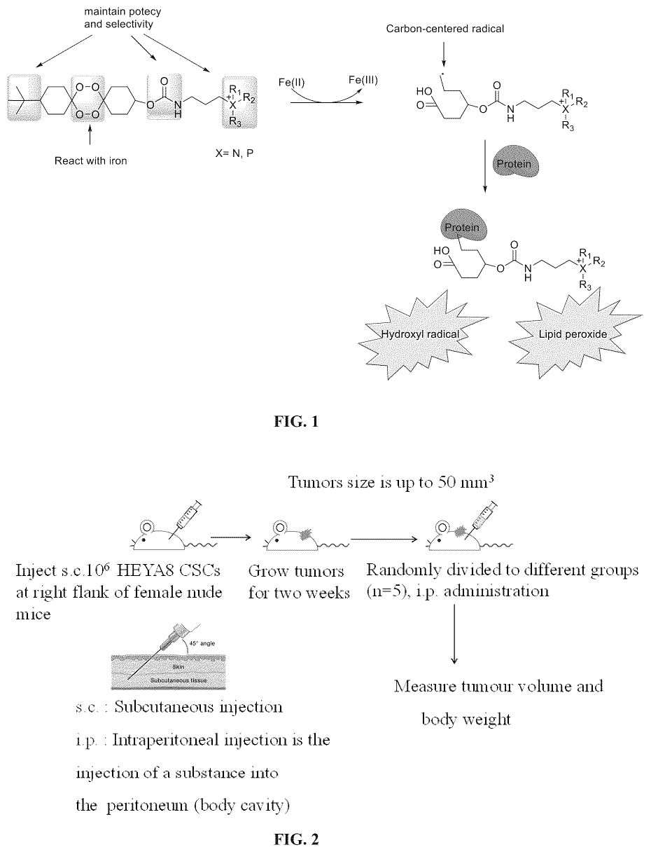

These exemplary compounds demonstrate that the cytotoxicity of 1,2,4,5-tetraoxanes can be tuned by changing the targeting group. 1,2,4,5-tetraoxanes with carbamate linker show superior activity than the amide one. Further, compounds with targeting groups gave better anticancer effect and selectivity. If the targeting group containing phosphonium or quaternary ammonium moieties, the cytotoxicity and selectivity of the invention will be greatly improved. The biological features of the exemplary compounds are shown in FIG. 1.

Generally, from the formula of “cyclic ring+1,2,4,5-tetraoxane+ targeting group”, the cyclic ring provide lipophilicity for cell permeability and the targeting group provides water solubility. For example, the tert-butyl cyclohexyl ring is identified as the component to maintain the lethality to cancer cells, and the phosphonium or quaternary ammonium groups are elements in the targeting groups to furnish cytotoxicity and selectivity.

### Example 2. The Compounds Show Anticancer Activities

Materials and Methods

The anticancer activities of the exemplary compounds listed in Example 1 were tested against human cervix Hela cells, mammary breast cancer MDA-MB-231 cells, human hepatocellular HepG2 cells, human colorectal HCT116 cells, mammary ductal T47D cells, non-cancerous NIH3T3 cells, dog's kidney MDCK cells, and mouse brain bEnd.3 cells, respectively.

Results

The viability test results of the compounds are summarized in Tables 1 to 12a. Generally, the tetraoxane compounds show broad-spectrum anticancer activities against colon HCT116, breast MDA-MB-231 and MCF-7, ovarian HEYA8, leukemia HL-60, liver Huh7 and PLC, and bone U2OS cancer cells. Erastin, RSL3, and artesunate are known compounds. However, their performance in breast cancer MDA-MB-231 cells and non-cancerous NIH3T3 cells has not been reported before. Table 12b summarizes some of the compounds that elicited good selectivity between cancer and non-cancerous cell lines. Further, this is the first report showing that 1,2,4,5-tetraoxane derivatives eradicate cancer cells selectively by ferroptosis pathway. Table 13 summaries IC₅₀ values and selectivity of compounds 37b and 48, in comparison with known drugs, i.e. OZ277, OZ439, and RKA 182. OZ422 and OZ439 (Vennerstrom et al.) belong to the class of 1,2,4-trioxolane but they illustrated poor activity against MDA-MB-231, HCT116 and HL-60 cells (general IC₅₀ values>10 μM).

Tetraoxane 37b, 48, 37f, 53, 56 and alk-R-48 generally have IC₅₀ values<4 μM against triple negative breast cancer MDA-MB-231 cells. They are at least 10-fold more cytotoxic to MDA-MB-231 cells than non-cancerous NIH3T3 and MDCK. For example, compound 48 is 12-fold, 13-fold, and 24-fold more cytotoxic towards breast cancer MDA-MB-231 cells (IC₅₀=2.4±0.7 μM), colon HCT116 cells (IC₅₀=2.2±0.3 μM), and leukemia HL-60 cells (IC₅₀=1.2±0.9 μM), respectively, than non-cancerous NIH3T3 cells (IC₅₀=29.0±5.7 μM). In contrast, erastin and RSL3 are more cytotoxic to non-cancerous cells than cancer cells, showing their lack of selectivity.

For reported compounds in the literature, such as OZ422, OZ439, and FINO₂, they illustrated poor activities against MDA-MB-231 cells (IC₅₀ values>10 βM, in general). Compounds 48 (IC₅₀ on MDA-MB-231: 2.4±0.7 μM) and 37b (IC₅₀ on MDA-MB-231: 0.4±0.05 μM) are 10-fold and 64-fold more cytotoxic than the literature example OZ439 on the same breast cancer cell line, respectively. Compound 37b is 87-fold more cytotoxic to breast cancer MDA-MB-231 cells than OZ277. Compounds 48 (IC₅₀ on HCT116: 2.2±0.3 μM) and 37b (IC₅₀ on HCT116: 0.6±0.3 μM) are 29-fold and 106-fold more cytotoxic than the literature example OZ439 on the same colon cancer cell line, respectively. Additionally, compounds 48 and 37b are 44-fold and 113-fold more cytotoxic to colon cancer HCT116 cells than OZ277, respectively. Compound 48 is at least 9-fold more cytotoxic to leukemia HL-60 cells than OZ439. FINO₂ (Woerpel et al.) belongs to the class of 1,2-dioxolane but it shows poor performance in TNBC MDA-MB-231 cells (IC₅₀>10 μM). Compounds 48 and 37b are at least 4-fold and 25-fold more potent than FINO₂ in eradicating MDA-MB-231 cells. It has been reported that FINO₂ induced ferroptosis, oxidized iron and targeted to GPX4 indirectly. The compounds disclosed herein can induce ferroptosis and oxidize iron, with no protein targets. Additionally, the compounds disclosed herein can kill CSCs with IC₅₀<2 μM.

RKA182 (reported compound in the literature) is a second example of 1,2,4,5-tetraoxane which shows inferior activity against HL-60 cells (IC₅₀=11.6±0.4 μM). In comparison with 1,2,4,5-tetraoxane RKA182, compound 48 is 9-fold more potent than RKA182 in leukemia HL-60 cells. The best selectivity index for RKA182 is 4 (PMBC: IC₅₀=46.6±0.7 μM versus HL-60: IC₅₀=11.6±0.4 μM). In contrast, compound 48 has selectivity up to 24-fold (NIH3T3: IC₅₀=29.0±5.7 μM versus HL-60: IC₅₀=1.2±0.9 μM). Further, RKA182 does not perform well in killing cervical cancer Hela cells, liver cancer HepG2 cells and kidney cancer HEK293 cells, with an overall IC₅₀>12 μM. In contrast, the exemplary compounds show an overall IC₅₀ values<3 μM in the above-mentioned cancer cell lines. In particular, for HepG2 cells, compound 37b is 36-fold more cytotoxic than RKA182. Further, it has been reported that RKA182 induced apoptosis in cancers, not ferroptosis. A second-generation of RKA182 has been published by O'Neill's group for treating malaria, with no report on its anticancer activity. Tetraoxane E209 is another example of targeting to malaria. However, no anticancer activity has been reported.

TABLE 1
IC₅₀ values (μM) of tetraoxanes against MDA-MB-231 cells

| Compound | IC₅₀ (μM) | Std Dev |
|---|---|---|
| 6b | 2.6 | 1.0 |
| 30a | 14.4 | 1.1 |
| 37 | 7.4 | 0.5 |
| 37a | 2.0 | 1.3 |
| 37b | 0.38 | 0.049 |
| 30b | 0.53 | 0.037 |
| 37f | 2.9 | 0.8 |
| 37g | 11.0 | 0.9 |
| 37i | 24.3 | 3.3 |
| 37j | 23.9 | 5.5 |
| 37d | 31.7 | 7.0 |
| 37e | 1.7 | 0.5 |
| 48 | 2.4 | 0.7 |
| 49 | 31.5 | 1.3 |
| 54 | 14.7 | 3.2 |
| 53 | 3.7 | 0.4 |
| 56 | 0.97 | 0.1 |
| 50 | 19.2 | 5.0 |
| 51 | 26.1 | 1.0 |
| 57 | 2.9 | 0.09 |
| 59a | 10.4 | 0.8 |
| 59b | 1.9 | 0.8 |
| alk-R-37b | 2.3 | 0.3 |
| alk-R-48 | 0.8 | 0.2 |
| alk-L-37b | 2.1 | 0.3 |
| alk-L-48 | 8.8 | 2.1 |
| 59 | >50 | Nil |
| alk-L-37 | 24.5 | 1.4 |
| Erastin | 1.3 | 0.2 |
| RSL3 | 0.041 | 0.015 |
| Artesunate | 20.3 | 3.5 |

^{a} IC₅₀ values were calculated from MTT assay concentration-response curves following incubation with the indicated compound for 48 h. Data represent mean ± SD, n = 3.

TABLE 2
IC₅₀ values (μM) of tetraoxanes against Hela cells

| Compound | IC₅₀ (μM) | Std Dev |
|---|---|---|
| 6b | 2.2 | 1.2 |
| 60a | 2.3 | 1.2 |
| 37 | 15.1 | 2.1 |
| 37a | 3.9 | 1.8 |
| 37b | 1.6 | 0.4 |
| 30b | 2.4 | 0.5 |
| 37f | 3.1 | 0.1 |
| 37g | 11.5 | 1.6 |
| 37i | 28.5 | 3.9 |
| 37j | 30.6 | 1.8 |
| 37d | 24.8 | 4.6 |
| 37e | 1.7 | 0.2 |
| 48 | 6.3 | 2.1 |

^{a} IC₅₀ values were calculated from MTT assay concentration-response curves following incubation with the indicated compound for 48 h. Data represent mean ± SD, n = 3.

TABLE 3
IC₅₀ values (μM) of tetraoxanes against T47D cells

| Compound | IC₅₀ (μM) | Std Dev |
|---|---|---|
| 6b | 4.4 | 1.9 |
| 30a | ND | ND |
| 37 | 6.1 | 0.9 |
| 37a | 2.2 | 0.3 |
| 37b | 0.62 | 0.11 |
| 30b | ND | ND |
| 37f | 1.3 | 0.6 |
| 37g | 3.1 | 0.7 |
| 37i | 13.2 | 2.8 |
| 37j | 10.6 | 1.3 |
| 37d | 10.4 | 4.1 |
| 37e | 1.2 | 0.5 |
| 48 | 1.7 | 0.3 |

^{a} IC₅₀ values were calculated from MTT assay concentration-response curves following incubation with the indicated compound for 48 h. Data represent mean ± SD, n = 3. ND = not determined.

TABLE 4
IC₅₀ values (μM) of tetraoxanes against HEK293 cells

| Compound | IC₅₀ (μM) | Std Dev |
|---|---|---|
| 6b | 1.6 | 0.8 |
| 30a | 2.5 | 0.6 |
| 37 | 5.8 | 0.1 |
| 37a | 0.8 | 0.2 |
| 37b | 1.6 | 0.6 |
| 30b | 1.0 | 0.2 |

^{a} IC₅₀ values were calculated from MTT assay concentration-response curves following incubation with the indicated compound for 48 h. Data represent mean ± SD, n = 3.

TABLE 5
IC₅₀ values (μM) of tetraoxanes against HepG2 cells

| Compound | IC₅₀ (μM) | Std Dev |
|---|---|---|
| 37 | 4.8 | 0.5 |
| 37a | 2.0 | 0.1 |
| 37b | 0.7 | 0.5 |
| 30b | 1.2 | 0.7 |

^{a} IC₅₀ values were calculated from MTT assay concentration-response curves following incubation with the indicated compound for 48 h. Data represent mean ± SD, n = 3.

TABLE 6
IC₅₀ values (μM) of tetraoxanes against Jurkat cells

| Compound | IC₅₀ (μM) | Std Dev |
|---|---|---|
| 48 | 1.0 | 0.4 |

^{a} IC₅₀ values were calculated from MTT assay concentration-response curves following incubation with the indicated compound for 48 h. Data represent mean ± SD, n = 3.

TABLE 7
IC₅₀ values (μM) of tetraoxanes against NIH3T3 cells

| Compound | IC₅₀ (μM) | Std Dev |
|---|---|---|
| 37 | 7.1 | 2.2 |
| 37a | 1.7 | 0.7 |
| 37b | 4.6 | 1.9 |
| 30b | 5.8 | 0.5 |
| 37f | 11.6 | 2.4 |
| 37g | 26.6 | 2.6 |
| 37i | >100 | ND |
| 37j | >100 | ND |
| 37d | 30.3 | 1.5 |
| 37e | 1.9 | 0.9 |
| 48 | 29.0 | 5.7 |
| 49 | >50 | ND |
| 54 | >50 | ND |
| 53 | 33.7 | 3.4 |
| 56 | 11.1 | 0.6 |
| 50 | 44.9 | 7.3 |
| 51 | >25 | ND |
| 57 | 9.8 | 1.4 |
| alk-R-37b | 9.7 | 3 |
| alk-R-48 | 10.8 | 3.7 |
| alk-L-37b | 12.9 | 1.4 |
| alk-L-48 | 18.2 | 3.8 |
| Erastin | 0.29 | 0.020 |
| RSL3 | 0.028 | 0.0086 |
| Artesunate | 17.4 | 7.2 |

^{a} IC₅₀ values were calculated from MTT assay concentration-response curves following incubation with the indicated compound for 48 h. Data represent mean ± SD, n = 3. ND = not determined.

TABLE 8
IC₅₀ values (μM) of tetraoxanes against HCT116 cells

| Compound | IC₅₀ (μM) | Std Dev |
|---|---|---|
| 37 | 14.4 | 5.6 |
| 37a | 4.6 | 0.9 |
| 37b | 0.6 | 0.3 |
| 30b | 1.4 | 0.9 |
| 48 | 2.2 | 0.3 |
| 49 | >50 | ND |
| 54 | 26.2 | 12.0 |
| 53 | 6.7 | 1.4 |
| 56 | 2.7 | 0.3 |
| 50 | >25 | ND |
| 51 | >50 | ND |

^{a} IC₅₀ values were calculated from MTT assay concentration-response curves following incubation with the indicated compound for 48 h. Data represent mean ± SD, n = 3. ND = not determined.

TABLE 9
IC₅₀ values (μM) of tetraoxanes against MDCK cells

| Compound | IC₅₀ (μM) | Std Dev |
|---|---|---|
| 6b | 18.1 | 1.7 |
| 30a | 42.8 | 2.9 |
| 37 | >50 | ND |
| 37a | 23.7 | 4.5 |
| 37b | 9.5 | 2.2 |
| 30b | 5.8 | 0.5 |
| 37f | 12.4 | 0.9 |
| 37g | 22.3 | 2.1 |
| 37i | >50 | ND |
| 37j | >100 | ND |
| 37d | >100 | ND |
| 37e | 19.3 | 4.7 |
| 48 | 28.2 | 3.8 |

^{a} IC₅₀ values were calculated from MTT assay concentration-response curves following incubation with the indicated compound for 48 h. Data represent mean ± SD, n = 3. ND = not determined.

TABLE 10
IC₅₀ values (μM) of tetraoxanes against HL-60 cells

| Compound | IC₅₀ (μM) | Std Dev |
|---|---|---|
| 48 | 1.2 | 0.9 |

^{a} IC₅₀ values were calculated from MTT assay concentration-response curves following incubation with the indicated compound for 48 h. Data represent mean ± SD, n = 3.

TABLE 11
IC₅₀ values (μM) of tetraoxanes against bEnd.3 cells

| Compound | IC₅₀ (μM) | Std Dev |
|---|---|---|
| 6b | 12.3 | 1.4 |
| 37 | >100 | ND |
| 37a | 10.9 | 0.8 |
| 37b | 5.2 | 0.3 |

^{a} IC₅₀ values were calculated from MTT assay concentration-response curves following incubation with the indicated compound for 48 h. Data represent mean ± SD, n = 3. ND = not determined.

TABLE 12a
IC₅₀ values (μM) of tetraoxanes against varies cell lines.

| | IC₅₀ (μM)^{a} | | | |
|---|---|---|---|---|
| Compound | Huh7 | PLC | U2OS | MCF7 |
| 37b | 3.8 ± 0.9 | 2.7 ± 0.02 | 3.4 ± 0.2 | 2.4 ± 1.7 |
| 48 | 5.2 ± 0.4 | 3.9 ± 0.6 | 4.4 ± 0.6 | 8.7 ± 4.1 |

TABLE 12b
IC₅₀ values and selectivity of compounds against varies cell lines.

| | IC₅₀ (μM)^{a} | | | | |
|---|---|---|---|---|---|
| | MDA- | | | | |
| Compound | MB-231 | T47D | HCT116 | HL-60 | NIH3T3 |
| 37b | 0.4 ± 0.05 | 0.6 ± 0.1 | 0.6 ± 0.3 | ND | 4.6 ± 1.9 |
| | (>11.5) | (>7.6) | (>7.6) | | |
| 48 | 2.4 ± 0.7 | 1.7 ± 0.3 | 2.2 ± 0.3 | 1.2 ± 0.9 | 29.0 ± 5.7 |
| | (>12) | (>17) | (>13) | (>24) | |
| 37f | 2.9 ± 0.8 | 1.3 ± 0.6 | ND | ND | 11.6 ± 2.4 |
| | (>4) | (>8.9) | | | |
| 37g | 11.0 ± 0.9 | 3.1 ± 0.7 | ND | ND | 26.6 ± 2.6 |
| | (>2.4) | (>8.5) | | | |
| 53 | 3.7 ± 0.4 | ND | 6.7 ± 1.4 | ND | 33.7 ± 3.4 |
| | (>9.1) | | (>5) | | |
| 56 | 0.97 ± 0.1 | ND | 2.7 ± 0.3 | ND | 11.1 ± 0.6 |
| | (>11.4) | | (>4.1) | | |
| Alk-R-48 | 0.8 ± 0.2 | ND | ND | ND | 10.8 ± 3.7 |
| | (>13) | | | | |

^{a}ND stands for not determined. The value inside the bracket refers to the selectivity index (dividing the IC₅₀ of NIH3T3 by the IC₅₀ on cancer cells). ND = not determined.

TABLE 13
IC₅₀ values and selectivity of compounds 37b and 48, in comparison
with OZ277, OZ439, and RKA 182, against varies cell lines.

| | IC₅₀ (μM)^{a} | | | | |
|---|---|---|---|---|---|
| | MDA- | | | | |
| Compound | MB-231 | T47D | HCT116 | HL-60 | NIH3T3 |
| 37h^{#} | 0.4 ± 0.05 | 0.6 ± 0.1 | 0.6 ± 0.3 | ND | 4.6 ± 1.9 |
| | (>11) | (>7) | (>7) | | |
| 48^{#} | 2.4 ± 0.7 | 1.7 ± 0.3 | 2.2 ± 0.3 | 1.2 ± 0.9 | 29.0 ± 5.7 |
| | (>12) | (>17) | (>13) | (>24) | |
| OZ277* | 35.2 | ND | 68.3 | 53.8 | ND |
| OZ439* | 25.6 | ND | 63.9 | 10.9 | ND |
| RKA182* | ND | ND | ND | 11.6 ± 0.4 | ND |

^{#}experimental data
*reported data of known compounds
^{a}IC₅₀ values (μmol/L) were calculated from MTT assay concentration-response curves following incubation with the indicated compound for 48 hrs. Data represent mean ± SD, n = 3. ND = not determined. The value inside the bracket refers to the selectivity index (dividing the IC₅₀ of NIH3T3 by the IC50 on cancer cells).

### Example 3. The Compounds Show Anticancer Stem Cell Effect

Materials and Methods

The cytotoxicity of 1,2,4,5-tetraoxanes 48 and 37b on cancer stem cell (CSC) model of HEY A8 CSCs and mouse xenograft ovarian tumors (FIG. 2) was examined. Nude mice were injected s.c. with HEYA8 CSC cells (106) and randomly allocated to one of the six treatment groups (n=4): (i) DMSO, (ii) Paclitaxel (PTX), 5 mg/kg, (iii) compound 37b, 5 mg/kg, (iv) compound 37b, 10 mg/kg, (v) compound 48, 5 mg/kg, and (vi) compound 48, 10 mg/kg. When control animals (DMSO) began to appear tumor ulceration (25 days), all animals in the experiment were sacrificed and photographed. It is known that ovarian CSCs have resistance to Taxol, so Taxol was used as negative control.

Results

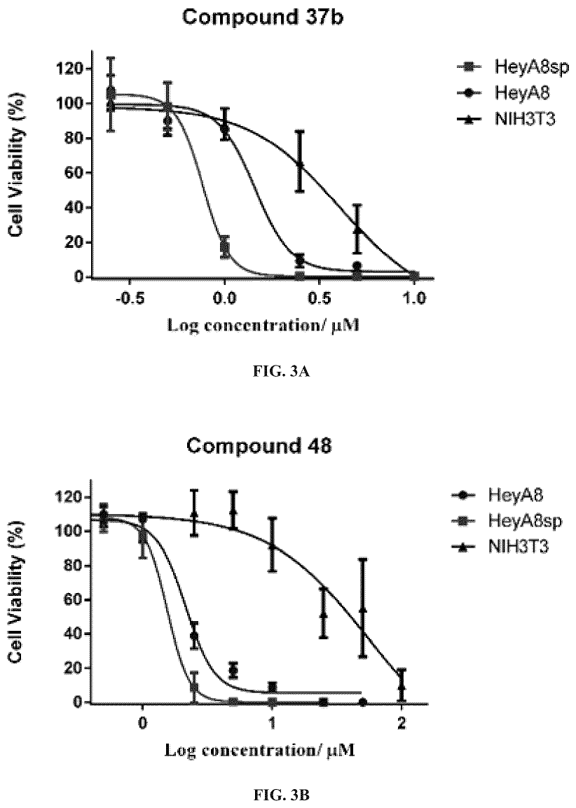

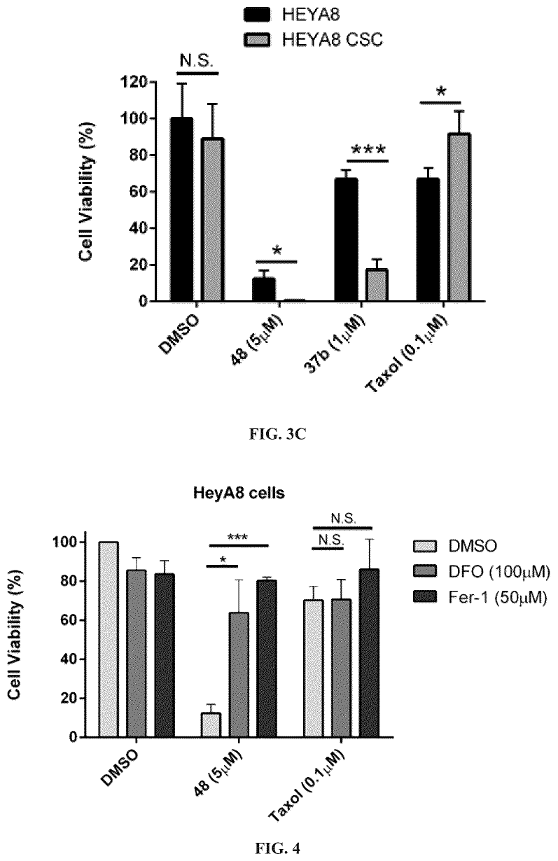

The IC₅₀ values of compounds 48 and 37b on adherent cancer cells HEY A8 were 2.3±0.7 μM and 1.4±0.07 μM, respectively (Table 14). Unlike Taxol, both peroxide compounds were able to eradicate almost all CSC spheroids (i.e. over 90%) at a concentration of 5 μM (FIGS. 3A and 3B). Of note, 5 μM of erastin and 50 μM of artesunate also did not show any cytotoxic effect on HEYA8 CSCs. This is the first report demonstrating that 1,2,4,5-tetraoxane derivatives can terminate cancer stem cells in micromole scale (eg. tetraoxane 48: IC₅₀=1.2±0.5 μM on HEYA8 CSCs). The 1,2,4,5-tetraoxane derivatives remove both cancer and CSCs simultaneously by the same small molecule.

TABLE 14
IC₅₀ values of compounds 48 and 37b against HeyA8 cells.

| | IC₅₀ (μM)^{a} | | |
|---|---|---|---|
| Compound | HeyA8 | HeyA8sp | NIH3T3 |
| 48 | 2.3 ± 0.7 | 1.2 ± 0.5 | 29.0 ± 5.7 |
| 37b | 1.4 ± 0.07 | 1.1 ± 0.4 | 4.6 ± 1.9 |

^{a}IC₅₀ values (μmol/L) were calculated from CellTiter-Glo ® assay concentration-response curves following incubation with the indicated compound for 48 hrs. Data represent mean ± SD, n = 3.

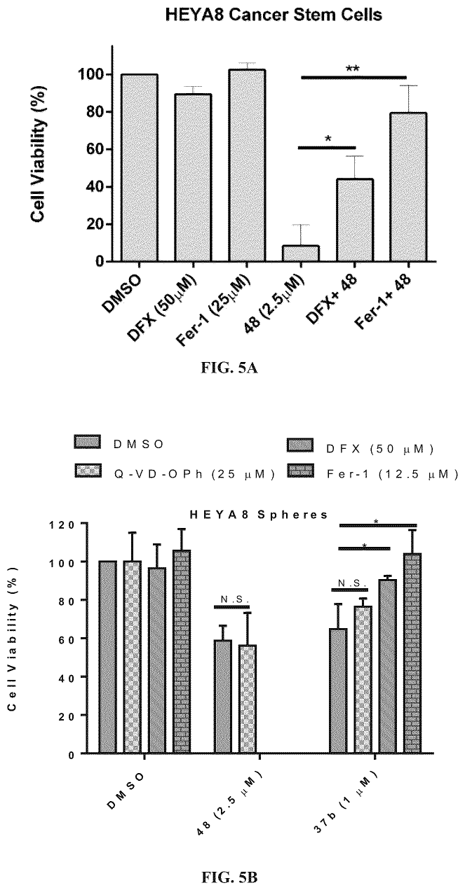

Pretreatment of adherent HEY A8 cells with iron chelator DFO or ferrostatin-1 (Fer-1) reversed the cell death induced by compound 48 but not by Taxol (FIG. 4). Lipophilic iron chelator deferasirox (DFX) was able to rescue part of the cell death in HEYA8 CSCs treated with compound 48 while the majority of the CSCs remained unaffected upon the co-treatment of compound 48 and Fer-1 (FIGS. 5A and 5B). It shows that compound 48 exerted its cytotoxicity in both cancer and CSCs by ferroptosis. Compound 37b also induced ferroptosis instead of apoptosis in HEYA8 CSCs. In contrast, known drugs, such as cholic acid, deoxycholic acid, and steroid derivatives of 1,2,4,5-tetraoxane have been reported to show anticancer effect. The cholic acid group carries a few protected polar groups which helps improving the bioavailability of the compound. However, the cholic acid derivative of tetraoxane as shown trigger off apoptosis in cancers. In contrast, the compounds disclosed herein produce hydroxyl radicals and lipid peroxides in cancer cells and induce ferroptosis. Structurally, the compounds disclosed herein differ from cholic acid, deoxycholic acid, and steroid derivatives of 1,2,4,5-tetraoxane in that the compounds here contain charged targeting groups like phosphonium or quaternary ammonium moieties for water solubility and do not contain two to three protected polar groups.

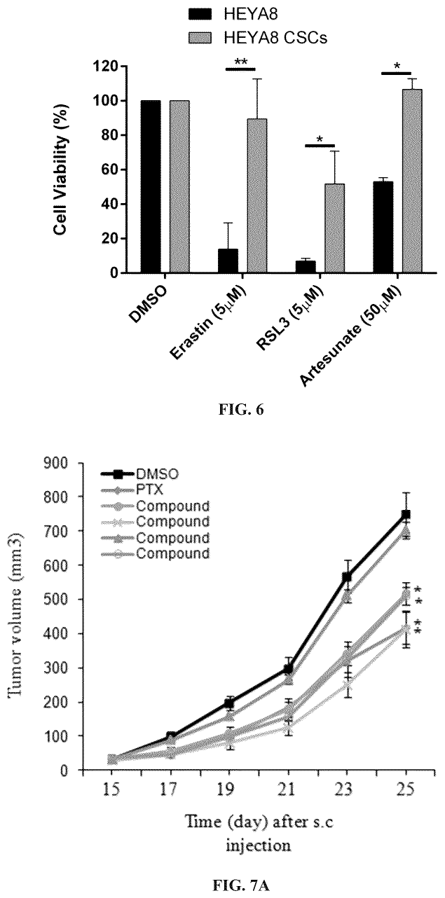

Erastin and RSL3 are selective agents for tumor cells bearing oncogenic RAS. However, their lethality towards CSCs is not well studied. While both erastin and RSL3 could eliminate the majority of adherent HEYA8 cells at 5 μM, high concentration of artesunate was required to achieve near 50% cell death (FIG. 6). On the contrary, HEYA8 CSCs were almost insensitive to erastin and artesunate at single dose, except for RSL3 (FIG. 6). GPX4 inhibition seems to be effective in suppressing the growth of CSCs. However, if consider the toxicity of RSL3 on NIH3T3 cells (IC₅₀=28.3±8.6 nM), there is no advantage of using RSL3 as anti-CSC agent due to the loss of selectivity. The compounds described herein not only eradicates HEYA8 cancer cells with IC₅₀<3 μM, but it also eliminates CSCs with IC₅₀<2 μM. In particular, compound 48 is at least 24-fold more cytotoxic to HEYA8 CSCs than non-cancerous cells. The disclosed compounds perform better than erastin, RSL3, Taxol and artesunate in terms of both the lethality and selectivity to CSCs.

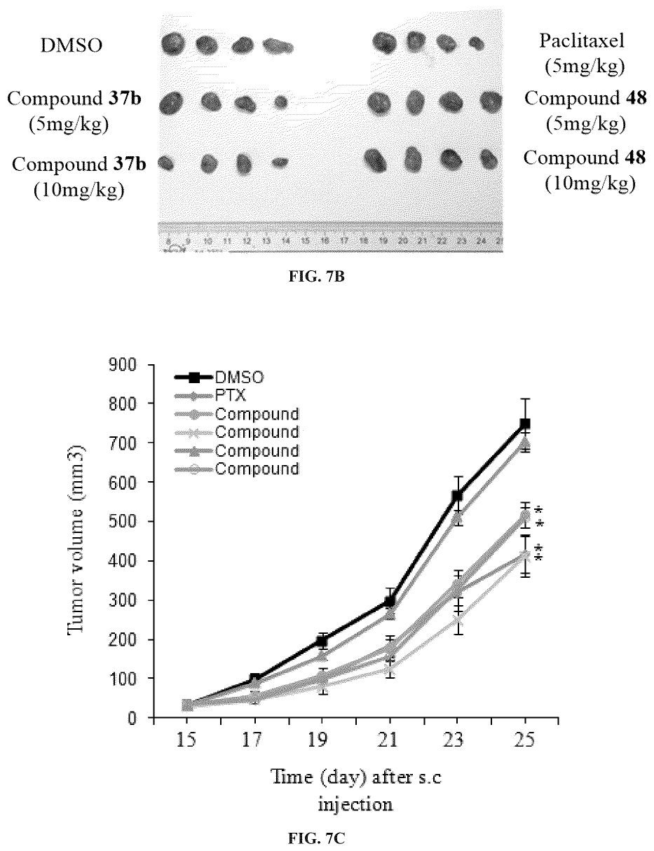

The compounds' anti-tumour ability was then evaluated using mouse xenograft ovarian tumors. Treatment with compound 37b (5 mg/kg and 10 mg/kg) alone was effective in inhibiting tumor growth compared with vehicle control (FIGS. 7A and 7B). Compound 48 (10 mg/kg) reduced the tumor size. In general, compounds 37b and 48 regimens were well-tolerated to the tested animals. There was no abnormal behavior or weight loss noted during the entire treatment period (FIG. 7C). The in vivo experiment successfully demonstrated the use of tetraoxane compounds as anti-cancer agents and guaranteed the safety profiles.

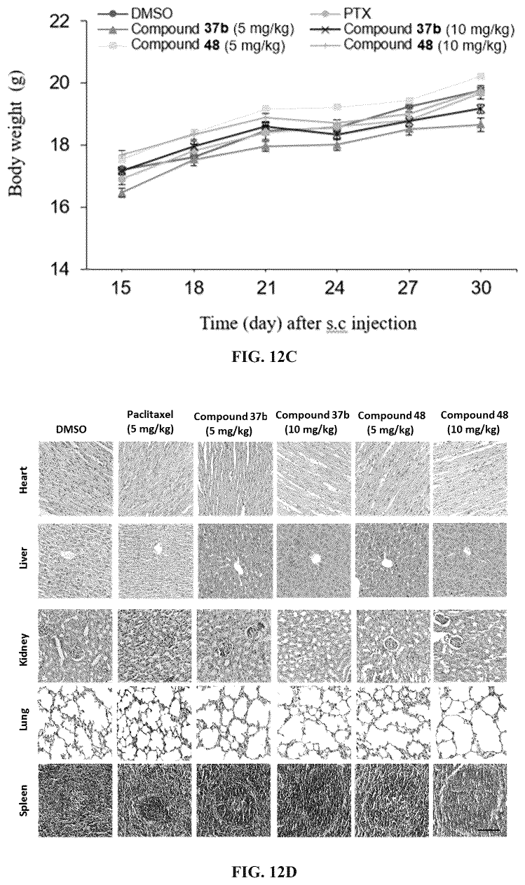

The compounds' anti-tumour ability was also evaluated using mouse xenograft breast tumors. Treatment with compound 37b (10 mg/kg) alone was effective in inhibiting tumor growth compared with vehicle control (FIGS. 12A and 12B). Compound 48 (5 mg/kg and 10 mg/kg) reduced the tumor size. There was no abnormal behavior or weight loss noted during the entire treatment period for both compounds 48 and 37c (FIG. 12C). Histopathological observations of the liver, heart, kidney, spleen, and lungs in the treated animals showed no histological alterations as compared to the controls (FIG. 12D).

### Example 4. Targeted Delivery of 1,2,4,5-Tetraoxanes Eradicated Cancer Cells by Ferroptosis

Materials and Methods

Fer-1 was used to investigate the cell death pathway induced by the compounds described herein. Liproxstatin-1 was used to further confirm whether 48 and 37b triggered off ferroptosis in MDA-MB-231 cells.

Results

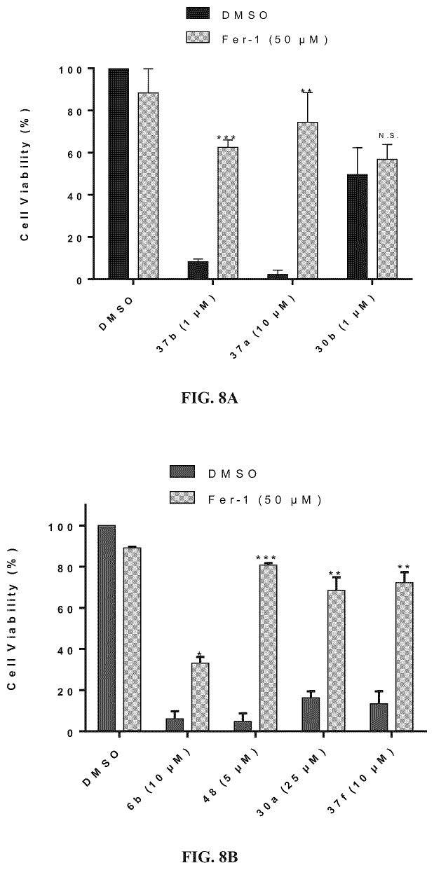

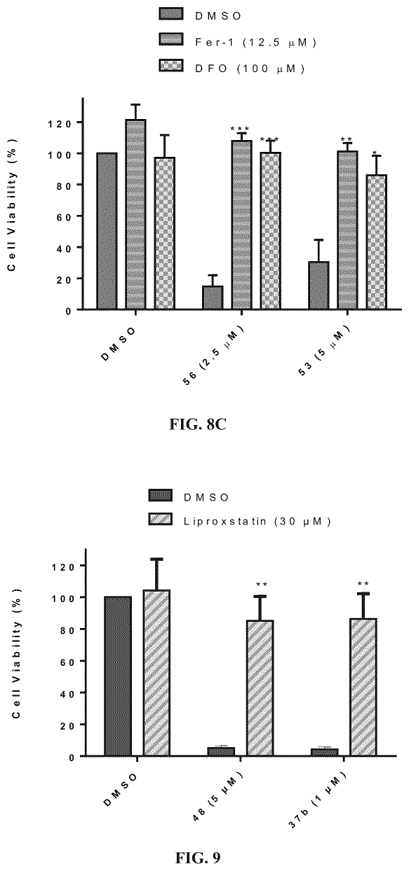

The viability result shows that Fer-1 subverted MDA-MB-231 cell death after treating with a number of tetraoxanes including 48 and 37b (FIGS. 8A-8C). Liproxstatin-1 was used to further confirm whether 48 and 37b triggered off ferroptosis in MDA-MB-231 cells. As shown in FIG. 9, pretreatment with this inhibitor in cells prevented the cell death caused by two tetraoxanes.

Reactive oxygen species is associated with the event of ferroptotic cell death. Oxidative stress induced by 48 and 37b can be indicated by the fluorogenic probe, HKOH-1r, that emits at 520 nm upon oxidation by cytosolic hydroxyl radical. By using HKOH-1r, it is possible to specify the type of ROS that tetraoxanes can produce in MDA-MB-231 cells.

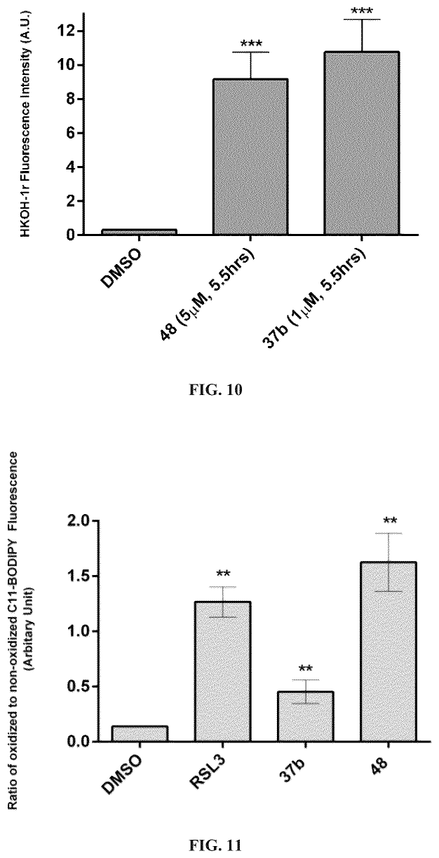

Confocal images of MDA-MB-231 cells after tetraoxane treatment were captured and the relative mean fluorescence intensities of cells with HKOH-1r were quantified (FIG. 10). The results show that significant amount of hydroxyl radical was observed about 5.5 h after the addition of 48 and 37b before significant cell death occurred. This phenomenon is consistent with one of the features of ferroptosis.

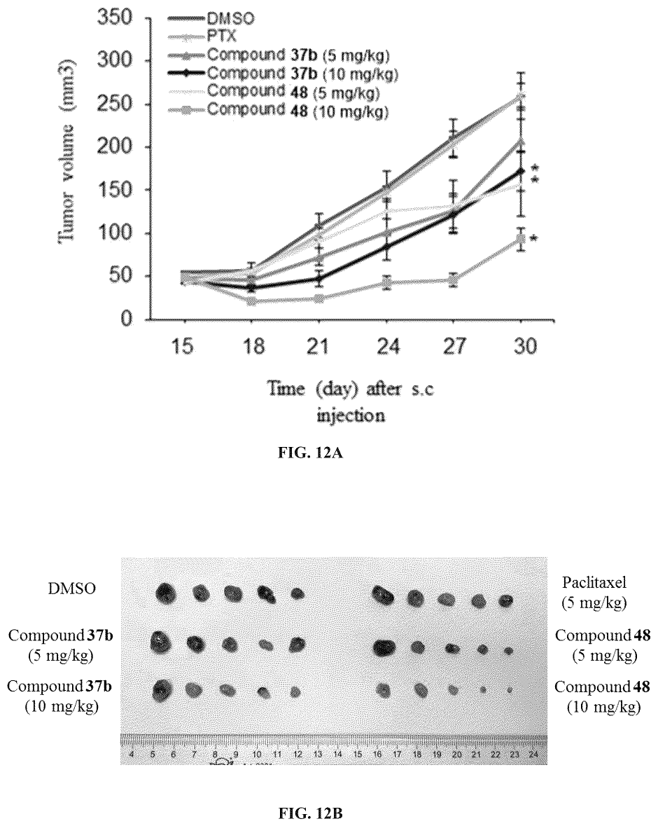

Since lipid peroxidation is the evidence about ferroptosis, C11-BODIPY probe is used to validate the lipid ROS generated by tetraoxanes. It is a ratiometric membrane-targeted probe which changes the emission from 590 nm to 510 nm upon the detection of lipid ROS. Here, MDA-MB-231 cells were treated with 48, 37b and RSL3, respectively. The lipid ROS levels were quantified by flow cytometry. Untreated group gave weak signal in FL1 channel (FIG. 11). This shows that the majority of C11-BODIPY conjugated system was intact without reacting with lipid ROS. For RSL3-treated group, fluorescence enhancement in FL1 was observed. Similar to RSL3, lipid ROS was generated in 48 or 37b-treated cells.

Some cancer cells can escape from the cytotoxicity of anticancer agents by upregulating some anti-apoptotic proteins, and thus develop resistance towards apoptotic cell death. In contrast, ferroptosis is an iron-dependent and reactive oxygen species (ROS)-dependent cell death pathway. It is known that cancer cells have elevated level of iron, which favours ferroptosis to take place to induce deleterious lipid peroxide and irreversible cell death, bypassing the anti-apoptotic pathways, and thus is advantageous. The data shown above has demonstrated that the tetraoxane compounds described herein eradicated both cancer cells and CSCs by ferroptosis.

The tetraoxane compounds described herein produce ROS inside the cancer cells for therapeutic purpose. Unlike photodynamic therapy, these tetraoxane compounds does not rely on any external irradiation for the ROS generation, which eliminates the issue associated with shallow penetration of the laser. Additionally, the effectiveness of treatment will not be affected by the intracellular pH. For example, the tetraoxane compounds generate hydroxyl radicals and lipid peroxide in breast cancer MDA-MB-231. This is advantageous because it is known that chemodynamic therapy (CDT) heavily relies on the acidity of the tumour microenvironment and high level of intracellular hydrogen peroxide to produce hydroxyl radicals which are strongly oxidizing agents. Sometimes CDT is ineffective if the tumour site is rich in blood supply and is neutral in pH. The concentration of H₂O₂ inside the cancer cells is not enough to produce lethal level of hydroxyl radicals for therapeutic purpose.

Further, the tetraoxane compounds described herein are small molecules that can be used in ferroptotic therapy, which eliminates the need for complex nanoparticle formulations encapsulating multiple components (eg. enzyme that catalyzes hydrogen peroxide production, iron oxide) for generating oxidative stress to ablate cancer cells.

### INDUSTRIAL APPLICABILITY

The invention provides an anticancer compound, which is useful in treating a cancer, reducing a cancer, or treating or ameliorating one or more symptoms associated with a cancer. Therefore, it may be prepared into corresponding medicament and has industrial applicability.

1.-40. (canceled)

41. A compound having the structure of 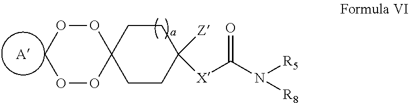
(a) wherein A′ is a substituted or unsubstituted C₃-C₃₀ polycycloalkyl, or a substituted or unsubstituted C₃-C₃₀ monocycloalkyl;
(b) wherein a is 1;
(c) wherein Z′ is a hydrogen;
(d) wherein X′ is O;
(e) wherein R₅ and R₈ are independently a hydrogen, a substituted or unsubstituted alkyl, or a substituted or unsubstituted alkynyl, or R₅ and R₈ taken together with the nitrogen to which they are attached form a substituted or unsubstituted heterocycloalkyl; and
(f) wherein the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group.

42. The compound of claim 41, wherein A′ is an adamantylidine, or a substituted or unsubstituted cyclohexyl.

43. The compound of claim 41,
wherein R₅ is hydrogen, and R₈ is a substituted or unsubstituted alkyl, or R₅ and R₈ taken together with the nitrogen to which they are attached form a substituted or unsubstituted heterocycloalkyl;
wherein the substituents are independently an amino, a phosphonium, or a sugar group.

44. The compound of claim 41 having the structure of 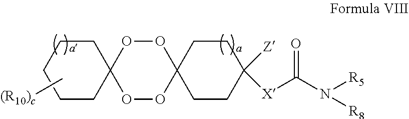
(a) wherein a is 1;
(b) wherein a′ is 1;
(c) wherein Z′ is a hydrogen;
(d) wherein X′ is O;
(e) wherein R₅ and R₈ are independently a hydrogen, a substituted or unsubstituted alkyl, or a substituted or unsubstituted alkynyl, or R₅ and R₈ taken together with the nitrogen to which they are attached form a substituted or unsubstituted heterocycloalkyl;
(f) wherein R₁₀ is a substituted or unsubstituted alkyl, and e is 0 or 1; and
(g) wherein the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group.

45. The compound of claim 44, wherein R₁₀ is a substituted or unsubstituted branched C₁-C₁₀ alkyl group; and wherein the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group.

46. The compound of claim 44, wherein R₁₀ is 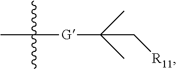
wherein G′ is 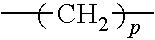
and p is 0; wherein R₁₁ is a hydrogen or 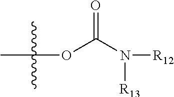
and R₁₂ and R₁₃ are independently a hydrogen, a substituted or unsubstituted alkyl, a substituted or unsubstituted alkenyl, or a substituted or unsubstituted alkynyl; and wherein the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group.

47. The compound of claim 46, wherein R₁₂ is a hydrogen and R₁₃ is a substituted or unsubstituted alkynyl.

48. The compound of claim 41, wherein R₅ is hydrogen and R₈ is 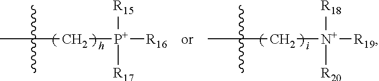
wherein h and i are independently an integer from 0 to 3; wherein R₁₅-R₂₀ are independently a hydrogen, a substituted or unsubstituted alkyl, a substituted or unsubstituted alkenyl, a substituted or unsubstituted alkynyl, a substituted or unsubstituted aryl, a substituted or unsubstituted heteroaryl, a substituted or unsubstituted polyaryl, a substituted or unsubstituted polyheteroaryl, a substituted or unsubstituted alkylaryl, or a substituted or unsubstituted arylalkyl.

49. The compound of claim 41 having the structure of

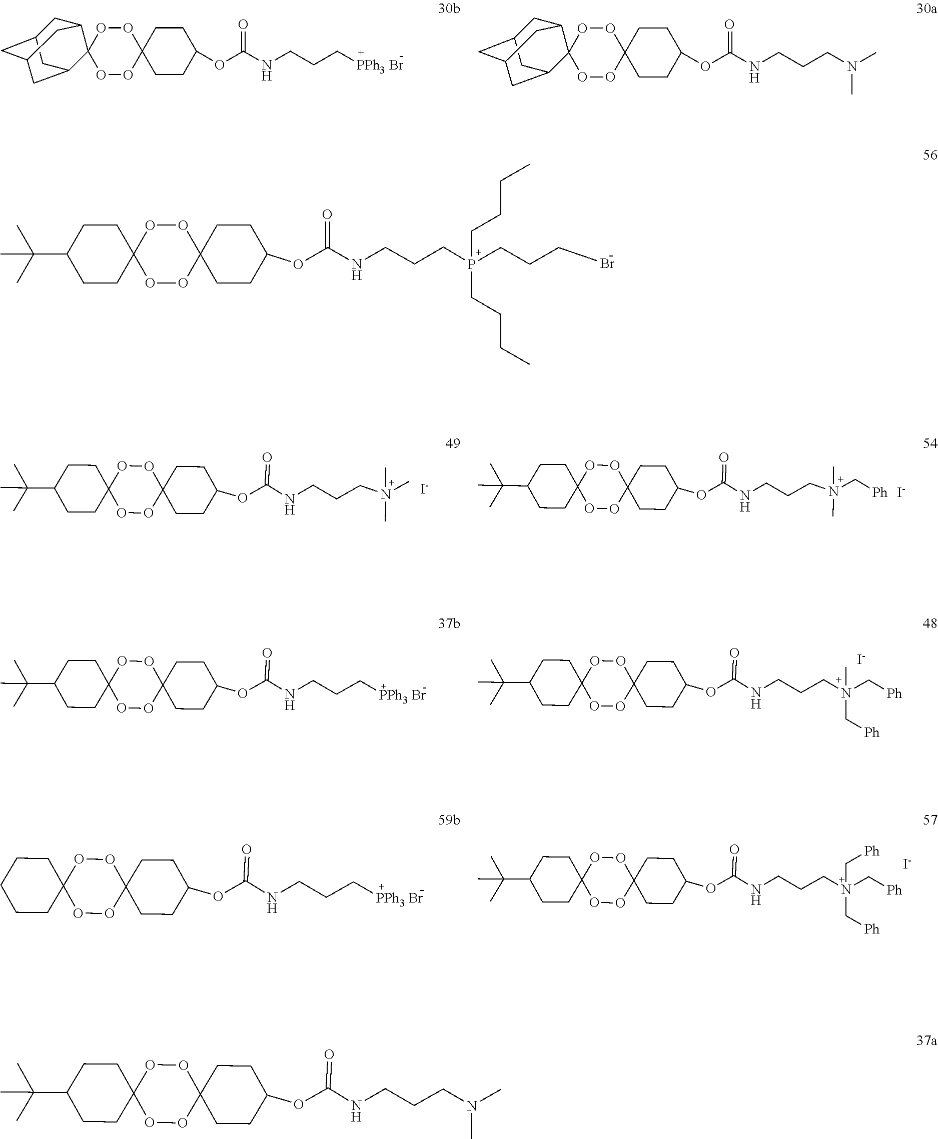

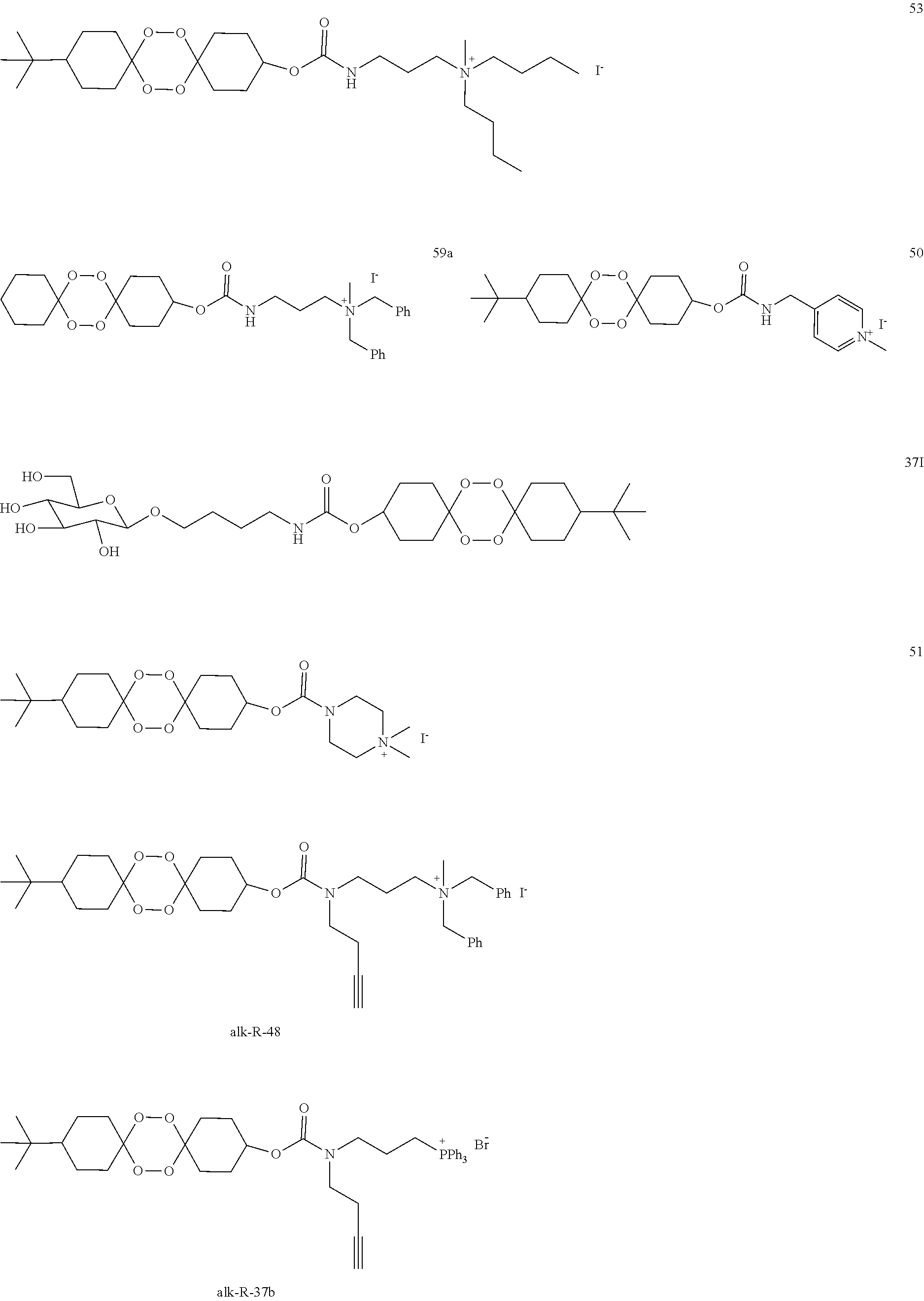

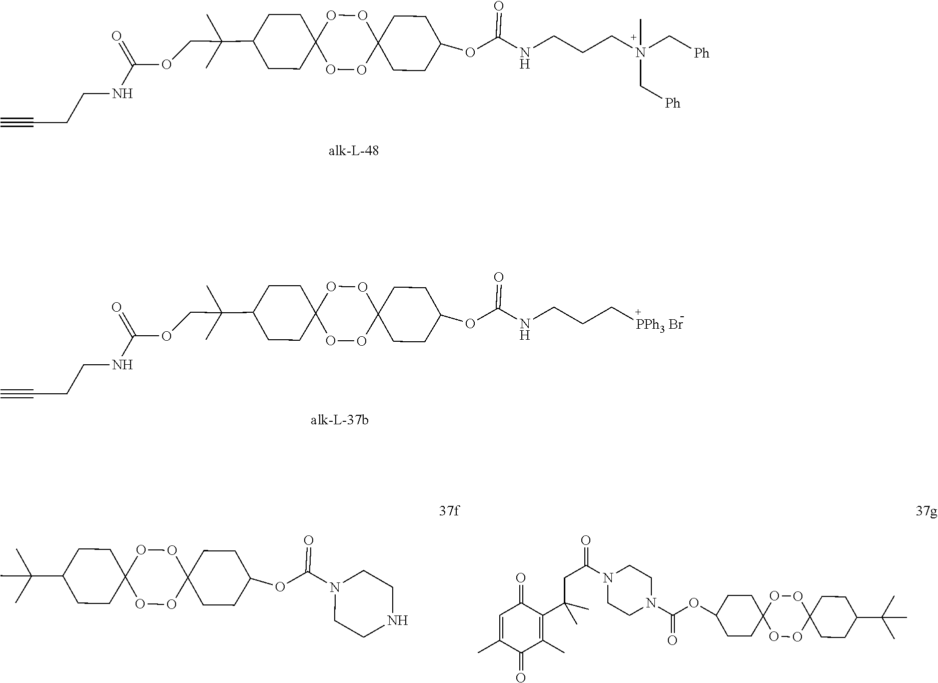

50. A compound having the structure of 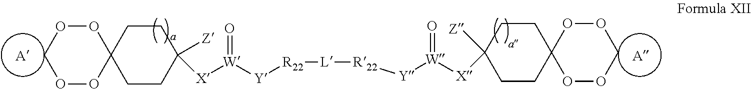
(a) wherein A′ and A″ are independently a substituted or unsubstituted C₃-C₃₀ polycycloalkyl, or a substituted or unsubstituted C₃-C₃₀ monocycloalkyl
(b) wherein a and a″ are 1;
(c) wherein Z′ and Z″ are hydrogen;
(d) wherein X′ and X″ are O;
(e) wherein W′ and W″ are C;
(f) wherein Y′ and Y″ are independently NR₈ or O;
(g) wherein R₂₂ and R′₂₂ are independently [symbol] CH₂ [symbol] ⱼ, and j is an integer from 0 to 3;
(h) wherein R₈ is a hydrogen;
(i) wherein L′ is a disulfide, or a polyether; and
(j) wherein the substituents are independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group.

51. The compound of claim 50, wherein A′ and A″ are independently an adamantylidine, or a substituted or unsubstituted cyclohexyl.

52. The compound of claim 50 having the structure of

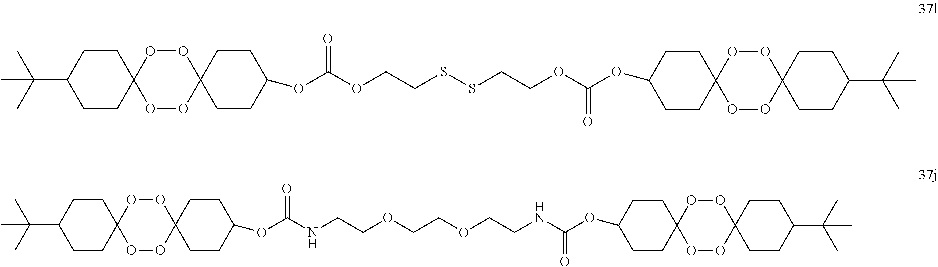

53. A pharmaceutical composition or dosage unit comprising
the compound of claim 41; and
a pharmaceutically acceptable excipient,
wherein the compound is in an effective amount to treat a cancer, reduce a cancer, or treat or ameliorate one or more symptoms associated with a cancer in a subject.

54. The pharmaceutical composition or dosage unit of claim 53 further comprising a second active agent, optionally more than one second active agent.

55. The pharmaceutical composition or dosage unit of claim 54, wherein the second active agent is an anticancer agent.

56. A method for treating a cancer, reducing a cancer, or treating or ameliorating one or more symptoms associated with a cancer in a subject in need thereof comprising
(i) administering to the subject an effective amount of the compound of claim 41 to treat the cancer, reduce the cancer, or treat or ameliorate one or more symptoms associated with the cancer in the subject.

57. The method of claim 56, wherein the subject is a mammal.

58. The method of claim 56, wherein the compound is administered by oral administration, parenteral administration, inhalation, mucosal administration, topical or a combination thereof.

59. The method of claim 56 further comprising administering to the subject a second active agent, optionally more than one second active agent, prior to, during, and/or subsequent to step (i).

60. The method of claim 59, wherein the second active agent is an anticancer agent.

61. The method of claim 56, wherein the cancer is colon cancer, breast cancer, ovarian cancer, cervical cancer, lung cancer, rectal cancer, kidney cancer, liver cancer, brain cancer, or leukemia, or a combination thereof.

62. A pharmaceutical composition or dosage unit comprising
the compound of claim 50; and
a pharmaceutically acceptable excipient,
wherein the compound is in an effective amount to treat a cancer, reduce a cancer, or treat or ameliorate one or more symptoms associated with a cancer in a subject.

63. The pharmaceutical composition or dosage unit of claim 62 further comprising a second active agent, optionally more than one second active agent.

64. The pharmaceutical composition or dosage unit of claim 63, wherein the second active agent is an anticancer agent.

65. A method for treating a cancer, reducing a cancer, or treating or ameliorating one or more symptoms associated with a cancer in a subject in need thereof comprising
(i) administering to the subject an effective amount of the compound of claim 50 to treat the cancer, reduce the cancer, or treat or ameliorate one or more symptoms associated with the cancer in the subject.

66. The method of claim 65, wherein the subject is a mammal.

67. The method of claim 65, wherein the compound is administered by oral administration, parenteral administration, inhalation, mucosal administration, topical or a combination thereof.

68. The method of claim 65 further comprising administering to the subject a second active agent, optionally more than one second active agent, prior to, during, and/or subsequent to step (i).

69. The method of claim 68, wherein the second active agent is an anticancer agent.

70. The method of claim 65, wherein the cancer is colon cancer, breast cancer, ovarian cancer, cervical cancer, lung cancer, rectal cancer, kidney cancer, liver cancer, brain cancer, or leukemia, or a combination thereof.
